(19) United States
(12) Patent Application Publication
(10) Pub. No.: US 2024/0300970 A1
(43) Pub. Date: Sep. 12, 2024

(54) LIGHT-EMITTING DEVICE AND DISPLAY DEVICE

(71) Applicant: BOE Technology Group Co., Ltd., Beijing (CN)

(72) Inventors: Lei Chen, Beijing (CN); Xueqin Chen, Beijing (CN); Bingyan Liang, Beijing (CN); Dongxu Zhang, Beijing (CN)

(73) Assignee: BOE Technology Group Co., Ltd., Beijing (CN)

(21) Appl. No.: 18/020,721

(22) PCT Filed: Mar. 31, 2022
(86) PCT No.: PCT/CN2022/084526

§ 371 Date: Feb. 10, 2023

(51) Int. Cl. C07D 493/04; C07D 403/14; C07D 491/048; C07D 495/04; C07D 519/00; H10K 50/84

(52) U.S. Cl. CPC C07D 493/04; C07D 403/14; C07D 491/048; C07D 495/04; C07D 519/00; H10K 50/84

(57) ABSTRACT

The present application provides a light-emitting device and a display device, which relates to the field of display technology, including: a substrate; a first electrode and a second electrode located on the substrate: a light-emitting layer located between the first electrode and the second electrode; and a capping layer located on a side of the second electrode away from the light-emitting layer, wherein the capping layer includes a compound of aromatic amines, and a structure of the compound of aromatic amines includes one of following two structures 1:

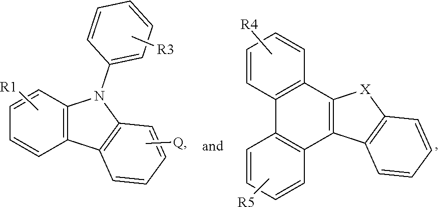

and the capping layer made of the compound of aromatic amines has a high refractive index and high stability, such that when the light-emitting device is used, the light-outlet efficiency of the device may be improved, and the service life of the device may be prolonged.

### CROSS-REFERENCE TO RELATED APPLICATION

The present disclosure is a National Stage of International Application No. PCT/CN2022/084526, filed on Mar. 31, 2022, with the title of “LIGHT-EMITTING DEVICE AND DISPLAY DEVICE”, which is incorporated herein in its entirety by reference.

### FIELD

The present application relates to the field of display technology, in particular to a light-emitting device and a display device.

### BACKGROUND

Organic light-emitting diode (OLED) display products have become hot mainstream display products in current market, due to their characteristics such as active lighting, high brightness, high resolution, wide viewing angle, fast response, low energy consumption, and flexibility. In related technology, light-outlet efficiency of OLED is improved and power consumption thereof is reduced by setting a capping layer on a light-outlet side of OLED display products.

### SUMMARY

Embodiments of the application adopts the following technical solutions:

In a first aspect of the disclosure, an embodiment of the disclosure provides a light-emitting device, including:
a substrate;
a first electrode and a second electrode, located on the substrate;
a light-emitting layer, located between the first electrode and the second electrode; and
a capping layer, located on a side of the second electrode away from the light-emitting layer, wherein the capping layer includes a compound of aromatic amines, and a structure of the compound of aromatic amines includes one of following two structures 1:

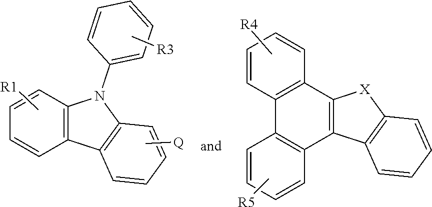

wherein the structure 1 is directly or indirectly bonded to a nitrogen atom in an amine of the compound of aromatic amines, the Q includes the R2 or a R2-substituted aryl group, the X includes one of C, O, N, NR6, and S, the R1, the R2, the R3, the R4, the R5 and the R6 respectively include one of: hydrogen, deuterium, halogen, a nitro group, a nitrile group, a substituted or unsubstituted C1 to C30 alkyl group, a substituted or unsubstituted C2 to C30 alkenyl group, a substituted or unsubstituted C1 to C30 alkoxy group, a substituted or unsubstituted C1 to C30 thioether group, a substituted or unsubstituted C6 to C50 aryl group, and a substituted or unsubstituted C2 to C50 heteroaryl group.

In some embodiments of the disclosure.
the structure

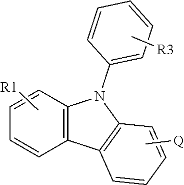

includes one of

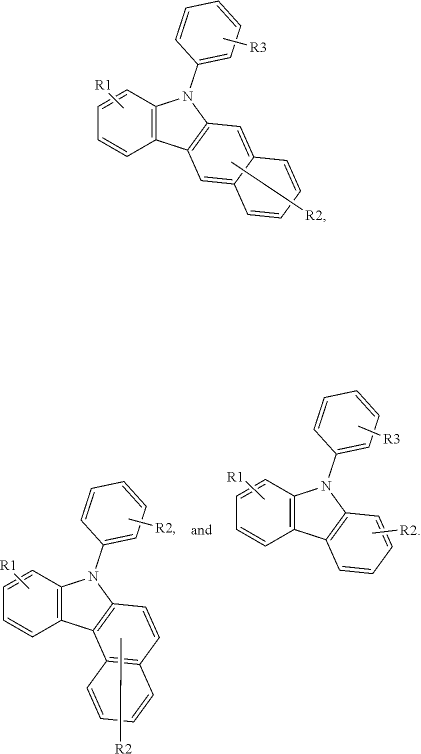

In some embodiments of the disclosure, the substituted or unsubstituted C2 to C50 heteroaryl group includes a substituted or unsubstituted C2 to C50 heteroaryl group formed by a substituted or unsubstituted ring structure of C2 to C9.

In some embodiments of the disclosure, the substituted C1 to C30 alkyl group, the substituted C2 to C30 alkenyl group, the substituted C1 to C30 alkoxy group, the substituted C1 to C30 thioether group, the substituted C6 to C50 aryl group, and the substituted C2 to C50 heteroaryl group are referred to being replaced by one or more of following groups: deuterium, halogen, a nitro group, a nitrile group, a C1 to C30 alkyl group, a C2 to C30 alkenyl group, a C1 to C30 alkoxy group, a C1 to C30 thioether group, a C6 to C50 aromatic group, and a C2 to C50 heteroaryl group.

In some embodiments of the disclosure, the compound of aromatic amines includes at least two heteroatoms.

In some embodiments of the disclosure, wherein the compound of aromatic amines further includes at least one of following four structures 2:

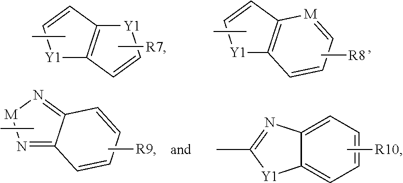

wherein the Y1 includes O or S, the M includes N or —CR11, and the R7, R8, R9, R10 and R11 respectively include one of: hydrogen, deuterium, halogen, a nitro group, a nitrile group, a substituted or unsubstituted C1 to C30 alkyl group, a substituted or unsubstituted C2 to C30 alkenyl group, a substituted or unsubstituted C1 to C30 alkoxy group, a substituted or unsubstituted C1 to C30 thioether group, a substituted or unsubstituted C6 to C50 aryl group, and a substituted or unsubstituted C2 to C50 heteroaryl group.

In some embodiments of the disclosure, a first position of one of the following three structures 1 is directly bonded with the nitrogen atom in the amine of the compound of aromatic amines, or
the first position of one of the following three structures 1 is bonded with the nitrogen atom in the amine of the compound of aromatic amines through L:

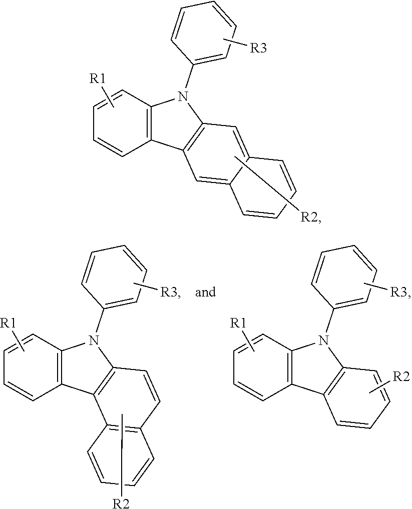

wherein the L is one of: the substituted or unsubstituted naphthyl group, a C6 to C50 arylidene group, and the substituted or unsubstituted C2 to C50 heteroaryl group, and
the first position is a position containing hydrogen or isotope of hydrogen.

In some embodiments of the disclosure, a second position of at least one of the following fourth structures 2 is directly bonded with the nitrogen atom in the amine of the compound of aromatic amines, or
the second position of at least one of the following fourth structures 2 is directly bonded with the nitrogen atom in the amine of the compound of aromatic amines through L:

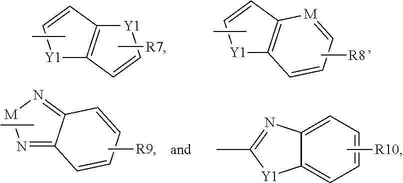

wherein the L is one of: the substituted or unsubstituted naphthyl group, the C6 to C50 arylidene group, and the substituted or unsubstituted C2 to C50 heteroaryl group, and

the second position is a position containing hydrogen or isotope of hydrogen.

In some embodiments of the disclosure, in a condition that the compound of aromatic amines includes the following structure 1, a general formula of the compound of aromatic amines is a following general formula I,

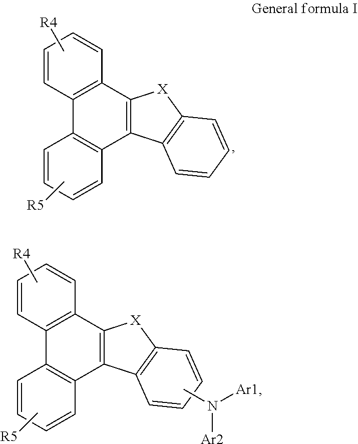

wherein a structure of at least one of the Ar1 and the Ar2 includes one of following three structures 3:

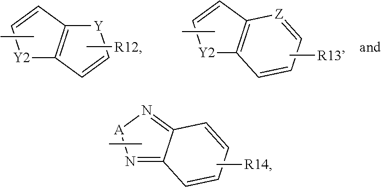

and
the structure 3 includes at least one heteroatom.

In some embodiments of the disclosure, the Y2 includes O, S or N, the Z includes C or N, and the A includes C or N, and

the R12, R13 and R14 respectively include one of: hydrogen, deuterium, halogen, a nitro group, a nitrile group, a substituted or unsubstituted C1 to C30 alkyl group, a substituted or unsubstituted C2 to C30 alkenyl group, a substituted or unsubstituted C1 to C30 alkoxy group, a substituted or unsubstituted C1 to C30 thioether group, a substituted or unsubstituted C6 to C50 aryl group, and a substituted or unsubstituted C2 to C50 heteroaryl group.

In some embodiments of the disclosure, the structure of another one of the Ar1 and Ar2 includes any one of: a nitrile group, a C2 to C30 alkyl group, a C3 to C30 naphthyl group, a substituted or unsubstituted C6 to C60 aryl group, and a substituted or substituted C5 to C60 heteroaryl group.

In some embodiments of the disclosure, a glass transition temperature of the compound of aromatic amines ranges from 130° C. to 160° C.

In some embodiments of the disclosure, a refractive index of the compound of aromatic amines at a wavelength of 460 nm ranges from 2.0 to 2.2,
the refractive index of the compound of aromatic amines at a wavelength of 530 nm ranges from 1.9 to 2.1, and
the refractive index of the compound of aromatic amines at a wavelength of 620 nm ranges from 1.85 to 2.0.

In some embodiments of the disclosure, the light-emitting device includes: the first electrode, a hole injection layer, a hole transport layer, an electron barrier layer, the light-emitting layer, a hole barrier layer, an electron transport layer, an electron injection layer, and the second electrode that are arranged by overlapping in sequence, and the first electrode is an anode and the second electrode is a cathode.

In some embodiments of the disclosure, a material of the hole injection layer includes at least one of a metal oxide, a material having hole transmission characteristics, and a P-type dopant: materials of the hole transport layer and the electron barrier layer respectively include one of a compound of aromatic amines, a compound of dimethylfluorenes, and a compound of carbazoles: materials of the hole barrier layer and the electron transport layer respectively include one of a derivative of imidazoles, a compound containing a structure of nitrogen six-membered ring, and a compound whose heterocycle has a substituent group of phosphine oxide series, and a material of the electron injection layer includes one of a metal or a metal compound.

In some embodiments of the disclosure, the light-emitting device further includes an encapsulation layer, wherein the encapsulation layer includes a glass encapsulation layer or a film encapsulation layer, and
in a condition that the encapsulation layer is the film encapsulation layer, a transition layer is further set between the capping layer and the film encapsulation layer, a material of the transition layer is an organic material, and a refractive index of the material of the transition layer is less than a refractive index of a material of the capping layer.

In a second aspect of the disclosure, an embodiment of the disclosure provides a display device, including the above light-emitting device.

The above description is only an overview of the technical solutions of the application. In order to better understand the technical means of the application, so as to implement the technical means according to the contents of the specification, and in order to make the above and other purposes, features and advantages of the application more distinct and understandable, specific implementations of the application are listed below.

### BRIEF DESCRIPTION OF THE DRAWINGS

In order to more clearly illustrate technical solutions in embodiments of the disclosure or in prior art, the followings will briefly introduce drawings needed to be used in illustrating the embodiments or the prior art. Apparently, the drawings in the following description are only some embodiments of the disclosure. For those ordinary skilled in the field, they may further obtain other drawings according to the provided drawings without paying creative labor.

FIG. 1 is a structural formula of a group of a derivative of carbazoles included in a compound of aromatic amines provided by an embodiment of the present application.

FIG. 2 shows a structural formula of a group of a derivative of phenanthrenes included in a compound of aromatic amines provided by an embodiment of the present application.

FIG. 3 is a schematic diagram of a method for preparing a compound of aromatic amines provided by an embodiment of the present application.

FIG. 4 is a schematic diagram of a structure of a light-emitting device provided by an embodiment of the application.

### DETAILED DESCRIPTION OF THE EMBODIMENTS

The followings will describe the technical solutions in the embodiments of the application clearly and completely in combination with the drawings in the embodiments of the application. Apparently, the described embodiments are a part of the embodiments of the application, not all of the embodiments of the application. Based on the embodiments in the application, all other embodiments obtained by the ordinary skilled in the art without doing creative work, belong to the scope of protection in the application.

In the drawings, thicknesses of areas or layers may be exaggerated for clarity. The same reference numerals in the drawings represent the same or similar structures, so their detailed description will be omitted. In addition, the accompanying drawings are only schematic illustrations of the application, and are not necessarily drawn to scale.

Unless the context otherwise requires, in the entire description and claims, the term “including” is interpreted as open and inclusive, that is, “including, but not limited to”. In the description of the specification, the terms “an embodiment”, “some embodiments”, “an exemplary embodiment”, “an example”, “a specific examples” or “some examples” are intended to indicate that specific features, structures, materials or characteristics related to the embodiment or example are included in at least one embodiment or example of the application. The exemplary expression of the above terms is not necessarily indicative of the same embodiment or example. In addition, the specific features, structures, materials, or characteristics may be included in any one or more embodiments or examples in any suitable manner.

In recent years, organic electroluminescence displays, such as OLED displays, as a new type of flat panel displays, have gradually attracted more attention. Because of their characteristics such as active lighting, high brightness, high resolution, wide viewing angle, fast response, low energy consumption, and flexibility, organic electroluminescence displays have become hot mainstream display products in current market. With continuous development of display products, customers have higher and higher requirements for product resolution and lower and lower requirements for power consumption, thus it is necessary to develop high efficiency, low voltage and long-life light-emitting devices.

Optimization and performance improvement of light-emitting devices may be realized by changing materials of film layers in the devices and/or collocations of materials between the film layers. Among them, a light-extraction material in a capping layer may effectively improve light-coupling efficiency of the device. The capping layer is a kind of organic or inorganic transparent material layer with a high refractive index in OLED devices, which has no absorption in the visible light range. The light-emitting device with the capping layer may improve a light-outlet mode, to make light originally confined in the device be emitted out of the device, showing a higher light-extraction efficiency. The higher the refractive index of material of the capping layer, the more notable a light-extraction effect, and the better performance optimization of the device. In addition, an increase of absorption of ultraviolet light by the capping layer may protect the device from damage of the ultraviolet light on internal film layers.

Based on the above, the application proposes a new light-emitting device.

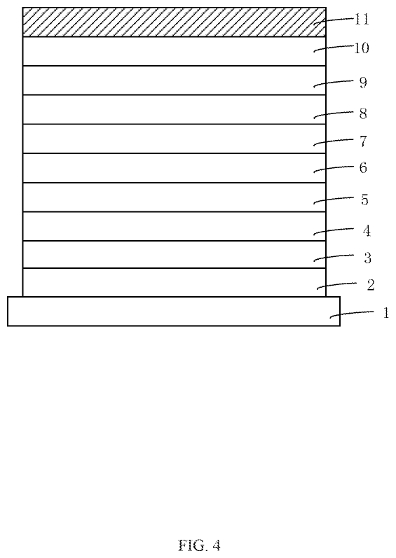

An embodiment of the present application provides a light-emitting device. Referring to that shown in FIG. 4, the light-emitting device includes: a substrate 1 and a first electrode 2 located on the substrate, a second electrode 10, a light-emitting layer 6 located between the first electrode 2 and the second electrode 10, and a capping layer 11. The capping layer 11 is located on a side of the second electrode 10 away from the light-emitting layer 6. The capping layer 11 includes a compound of aromatic amines, wherein a structure of the compound of aromatic amines includes one of the following two structures 1:

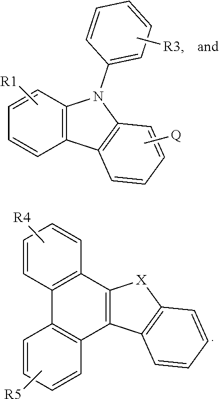

Among them, the structure 1 is directly or indirectly bonded to a nitrogen atom in an amine of the compound of aromatic amines. The Q includes the R2 or a R2-substituted aryl group. The X includes one of C, O, N, NR6, and S. The R1, the R2, the R3, the R4, the R5 and the R6 respectively include one of: hydrogen, deuterium, halogen, a nitro group, a nitrile group, a substituted or unsubstituted C1 to C30 alkyl group, a substituted or unsubstituted C2 to C30 alkenyl group, a substituted or unsubstituted C1 to C30 alkoxy group, a substituted or unsubstituted C1 to C30 thioether group, a substituted or unsubstituted C6 to C50 aryl group, and a substituted or unsubstituted C2 to C50 heteroaryl group.

In an exemplary embodiment, the

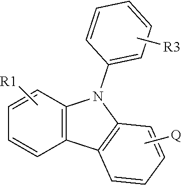

is a derivative of carbazoles, wherein a structure of carbazole is

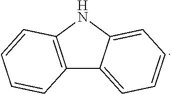 [SMILES: c1ccc2c(c1)[nH]c1ccccc12]

In an exemplary embodiment, the

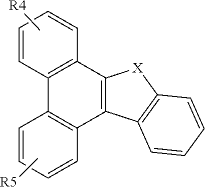

is a derivative of phenanthrenes, wherein a structure of phenanthrene is

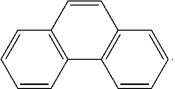 [SMILES: c1ccc2c(c1)ccc1ccccc12]

In some embodiments of the present application, the structure of

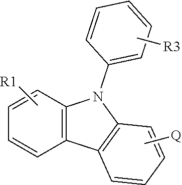

includes: one of

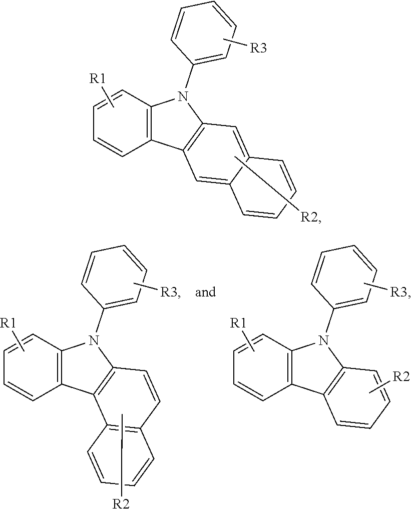

In an exemplary embodiment, Q includes the R2 or the R2-substituted aryl group. In a case that Q includes the R2, the structure

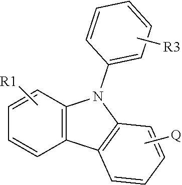

includes

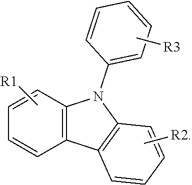

In a case that Q includes the R2-substituted aryl group, the structure

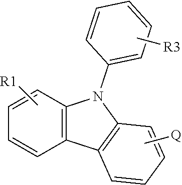

includes

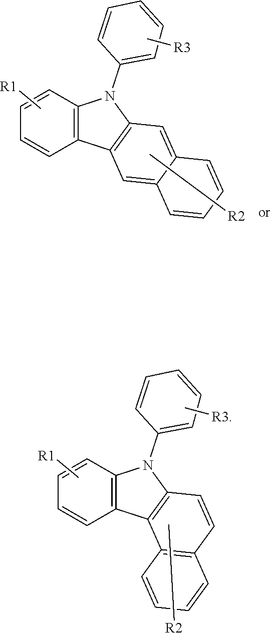

In an exemplary embodiment, the aryl group refer to a functional group or a substituent group derived from an aromatic ring.

For example, the aryl group includes, but is not limited to, a phenyl group, a naphthyl group, an anthracyl group, an acenaphthenyl group, an indenyl group, a phenanthryl group, an azulene group, a pyrenyl group, a fluorenyl group, a perylene group, a spirofluorenyl group, a spirodifluorenyl group, an yl group, a benzophenyl group, a benzoanthracene group, a fluoranthenyl group, a vinyl group, a tetraphenyl group, or an indenyl group.

In an exemplary embodiment, the heteroaryl group means that at least one carbon atom in an aromatic ring is replaced by a heteroatom. The heteroatom includes, but is not limited to, a nitrogen atom (N), a sulfur atom (S) or an oxygen atom (O). For example, the heteroaryl group includes at least one of: the nitrogen atom, the sulfur atom and the oxygen atom.

A number of the heteroatoms included in the heteroaryl group is not limited here, which may be determined according to an actual situation.

For example, the heteroaryl group includes, but is not limited to, a benzoxazolyl group, a benzothiazolyl group, an indolyl group, a benzimidazolyl group, a pyrrolyl group, a pyridyl group, a pyrimidinyl group, a pyrazinyl group, a pyridazinyl group, a triazinyl group, a tetrazinyl group, an imidazolyl group, a pyrazolyl group, a carbazolyl group, a thiophenyl group, a thiazolyl group, a benzocarbazolyl group, a dibenzocarbazolyl group, an indolecarbazolyl group, an indocarbazole group, a quinolinyl group, an isoquinolinyl group, a phthalazinyl group, a quinoxalinyl group, a misolinyl group, a quinazolinyl group, a phthalazinyl group, a benzoquinolinyl group, a benzoisoquinolinyl group, a benzoqunazolinyl group, a benzoquinoxalinyl group, an acridine group, a phenanthrolinyl group, a furanyl group, a pyranoyl group, an oxazinyl group, an oxazolyl group, an oxadiazolyl group, a triazolyl group, a dioxinyl group, a benzofuranyl group, a dibenzofuranyl group, a thiopyranoyl group, a thiazinyl group, a phenthio group, and a N-substituted spirofluorenyl group.

In some embodiments of the present application, the substituted or unsubstituted C2 to C50 heteroaryl group includes a C2 to C50 heteroaryl group formed by a substituted or unsubstituted ring structure of C2 to C9.

In some embodiments, in a condition that the R1, R2 and R3 include the substituted or unsubstituted C2 to C50 heteroaryl group, the substituted or unsubstituted C2 to C50 heteroaryl group includes the C2 to C50 heteroaryl group formed by the substituted or unsubstituted ring structure of C2 to C9.

In some embodiments, in a condition that the R4, R5 and R6 include the substituted or unsubstituted C2 to C50 heteroaryl group, a specific structure of the substituted or unsubstituted C2 to C50 heteroaryl group is not limited here.

Whether the above R1, R2, R3, R4. R5 and R6 are the same is not limited. In some embodiments, a part of the R1, R2, R3, R4, R5 and R6 may be the same. In some embodiments, structures of the R1, R2, R3, R4, R5 and R6 are different.

In some embodiments of the present application, the substituted C1 to C30 alkyl group, the substituted C2 to C30 alkenyl group, the substituted C1 to C30 alkoxy group, the substituted C1 to C30 thioether group, the substituted C6 to C50 aryl group, and the substituted C2 to C50 heteroaryl group are referred to being replaced by one or more of the following groups: deuterium, halogen, a nitro group, a nitrile group, a C1 to C30 alkyl group, a C2 to C30 alkenyl group, a C1 to C30 alkoxy group, a C1 to C30 thioether group, a C6 to C50 aromatic group, and a C2 to C50 heteroaryl group.

In some embodiments of the present application, the compound of aromatic amines includes at least two heteroatoms. For example, a plurality of heteroatoms included in the compound of aromatic amines may the same heteroatom, for example, the compound of aromatic amines may include a plurality of nitrogen atoms (N). For example, the plurality of heteroatoms included in the compound of aromatic amines may be a plurality of kinds of heteroatoms. For example, the compound of aromatic amines may include at least one nitrogen atom (N) and at least one of oxygen atom (O) or sulfur atom (S).

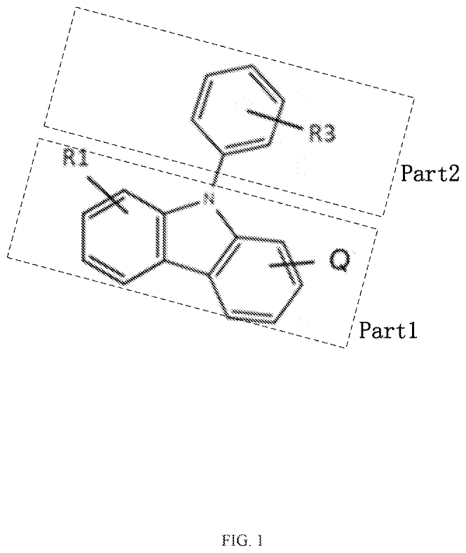

In an exemplary embodiment, referring to that shown in FIG. 1, the structure

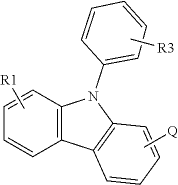

includes a first part (Part 1) and a second (Part 2), wherein the Part 1 may be a planar structure, occupy a small space and may increase a number of molecules per unit volume. In addition, the Part 1 has good conjugation, which may improve polarizability of the compound of aromatic amines, and help to improve a refractive index of material. There is a dihedral angle between the Part 2 and the Part 1, and the Part 2 may optimize a crystal structure when the compound of aromatic amines is crystallized, which makes the compound of aromatic amines have a high refractive index, and at the same time, have good processability and process stability during processing. Among them, good processability means easy preparation or high stability during preparation.

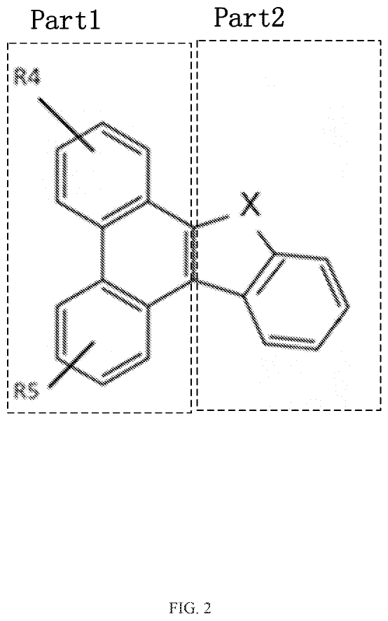

In the exemplary embodiment, referring to that shown in FIG. 2, the structure

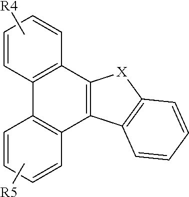

includes a first part (Part 1) and a second part (Part 2), wherein the Part 1 includes a structure of phenanthrene, the phenanthrene is formed by fusing three benzene rings and has a 180° planar conjugate structure, and under light stimulation, the polarizability of the compound is large, which is conducive to improvement of the refractive index of the material. The Part 2 includes a benzo five-membered ring fused on the phenanthrene, and the Part 2 improves a conjugation degree of the material, which makes a density of electron cloud in the compound of aromatic amines be further increased. After the density of electron cloud is increased, the electron cloud is easier to deform, which further increases the polarizability of the compound, and then improves the refractive index of the material.

In some embodiments of the present application, the compound of aromatic amines further includes at least one of the following four structures 2:

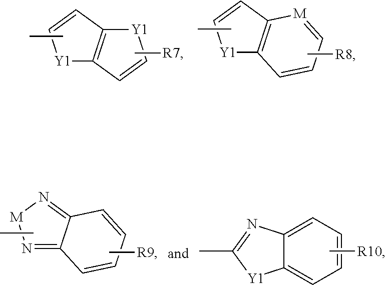

Among them, the Y1 includes O or S. The M includes N or —CR11. The R7, R8, R9, R10 and R11 respectively include one of: hydrogen, deuterium, halogen, a nitro group, a nitrile group, a substituted or unsubstituted C1 to C30 alkyl group, a substituted or unsubstituted C2 to C30 alkenyl group, a substituted or unsubstituted C1 to C30 alkoxy group, a substituted or unsubstituted C1 to C30 thioether group, a substituted or unsubstituted C6 to C50 aryl group, and a substituted or unsubstituted C2 to C50 heteroaryl group.

In an exemplary embodiment, the aryl group refers to the functional group or the substituent group derived from the aromatic ring.

For example, the aryl group includes, but is not limited to, the phenyl group, the naphthyl group, the anthracyl group, the acenaphthenyl group, the indenyl group, the phenanthryl group, the azulene group, the pyrenyl group, the fluorenyl group, the perylene group, the spirofluorenyl group, the spirobifluorenyl group, the yl group, the benzophenyl group, the benzanthracene group, the fluoranthenyl group, the vinyl group, the tetraphenyl group, and the indenyl group.

In an exemplary embodiment, the heteroaryl group means that at least one carbon atom in the aromatic ring is replaced by the heteroatom. The heteroatom includes, but is not limited to, the nitrogen atom, the sulfur atoms or the oxygen atom. The heteroaryl group includes at least one of: the nitrogen atom, the sulfur atom and oxygen atom.

The number of heteroatoms included in the heteroaryl group is not limited here, which may be determined according to an actual situation.

For example, the heteroaryl group includes, but is not limited to, the benzoxazolyl group, the benzothiazolyl group, the indolyl group, the benzimidazolyl group, the pyrrolyl group, the pyridyl group, the pyrimidinyl group, the pyrazinyl group, the pyridazinyl group, the triazinyl group, the tetrazinyl group, the imidazolyl group, the pyrazolyl group, the carbazolyl group, the thiophenyl group, the thiazolyl group, the benzocarbazolyl group, the dibenzcarbazolyl group, the indolecarbazolyl group, the indocarbazolyl group, the quinolinyl group, the isoquinolinyl group, the phthalazinyl group, the quinoxalinyl group, the misolinyl group, the quinazolinyl group, the phthalazinyl group, the benzoquinolinyl group, the benzoisoquinolinyl group, the benzoquinazolinyl group, the benzoquinoxalinyl group, the acridine group, the phenanthrolinyl group, the furanyl group, the pyranoyl group, the oxazinyl group, the oxazolyl group, the oxadiazolyl group, the triazolyl group, the dioxinyl group, the benzofuranyl group, the dibenzofuranyl group, the thiopyranoyl group, the thiazinyl group, the phenthio group and the N-substituted spirofluorenyl group.

In some embodiments of the present application, a first position of one of the following three structures 1 is directly bonded with the nitrogen atom in the amine of the compound of aromatic amines, or
the first position of one of the following three structures 1 is bonded with the nitrogen atom in the amine of the compound of aromatic amines through L.

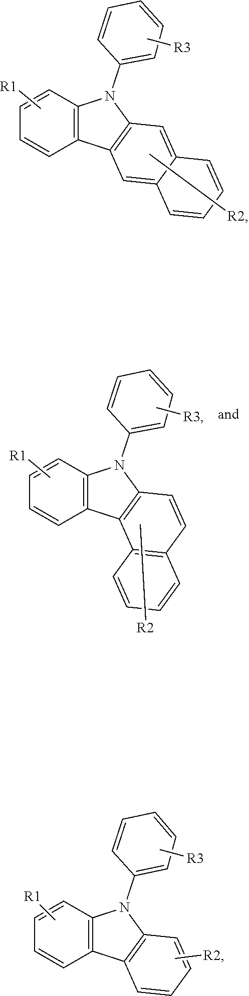

wherein the L is one of: the substituted or unsubstituted naphthyl group, a C6 to C50 arylidene group, and the substituted or unsubstituted C2 to C50 heteroaryl group. The first position is a position containing hydrogen or isotope of hydrogen.

In some embodiments of the present application, a second position of at least one of the following fourth structures 2 is directly bonded with the nitrogen atom in the amine of the compound of aromatic amines, or
the second position of at least one of the following fourth structures 2 is directly bonded with the nitrogen atom in the amine of the compound of aromatic amines through L,

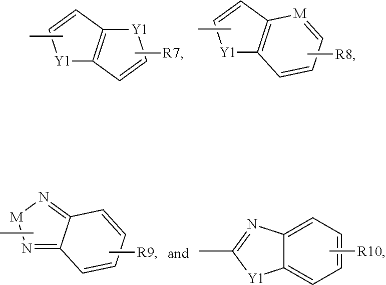

wherein the L is one of: the substituted or unsubstituted naphthyl group, the C6 to C50 arylidene group, and the substituted or unsubstituted C2 to C50 heteroaryl group. The second position is a position containing hydrogen or isotope of hydrogen.

A substituted position in the substituted naphthalene group is no limited here, and a specific structure of a substituted group in the substituted naphthalene group is no limited here either, which may be determined according to an actual situation.

A substituted position in the substituted C2 to C50 heteroaryl group is no limited here, and a specific structure of a substituted group in the substituted C2 to C50 heteroaryl group is no limited here either, which may be determined according to an actual situation.

For example, the compound of aromatic amines (containing a group of a derivative of carbazoles) may include any of the following structures:

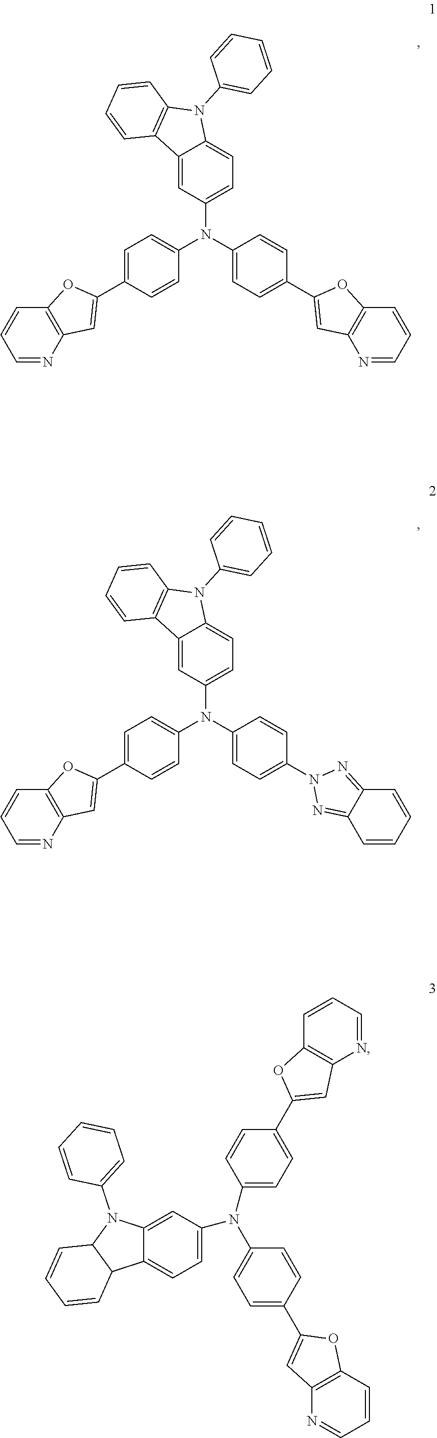 [SMILES: c1ccc(-n2c3ccccc3c3cc(N(c4ccc(-c5cc6ncccc6o5)cc4)c4ccc(-c5cc6ncccc6o5)cc4)ccc32)cc1 ; c1ccc(-n2c3ccccc3c3cc(N(c4ccc(-c5cc6ncccc6o5)cc4)c4ccc(-n5nc6ccccc6n5)cc4)ccc32)cc1 ; C1=CC2c3ccc(N(c4ccc(-c5cc6ncccc6o5)cc4)c4ccc(-c5cc6ncccc6o5)cc4)cc3N(c3ccccc3)C2C=C1]

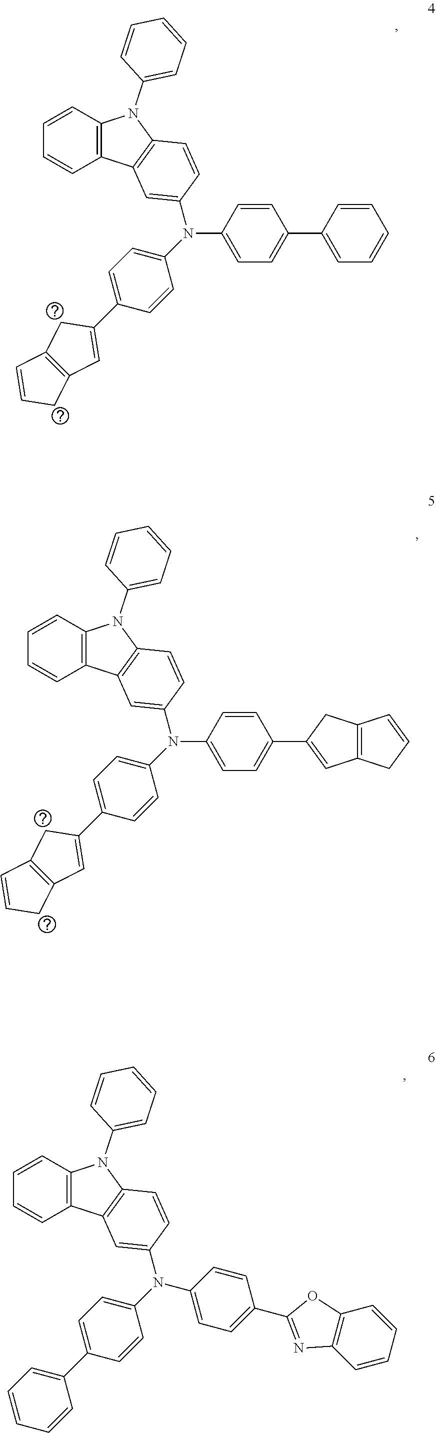 [SMILES: C1=CC2=C(C=C(c3ccc(N(c4ccc(-c5ccccc5)cc4)c4ccc5c(c4)c4ccccc4n5-c4ccccc4)cc3)C2)C1 ; C1=CC2=C(C=C(c3ccc(N(c4ccc(C5=CC6=C(C=CC6)C5)cc4)c4ccc5c(c4)c4ccccc4n5-c4ccccc4)cc3)C2)C1 ; c1ccc(-c2ccc(N(c3ccc(-c4nc5ccccc5o4)cc3)c3ccc4c(c3)c3ccccc3n4-c3ccccc3)cc2)cc1]

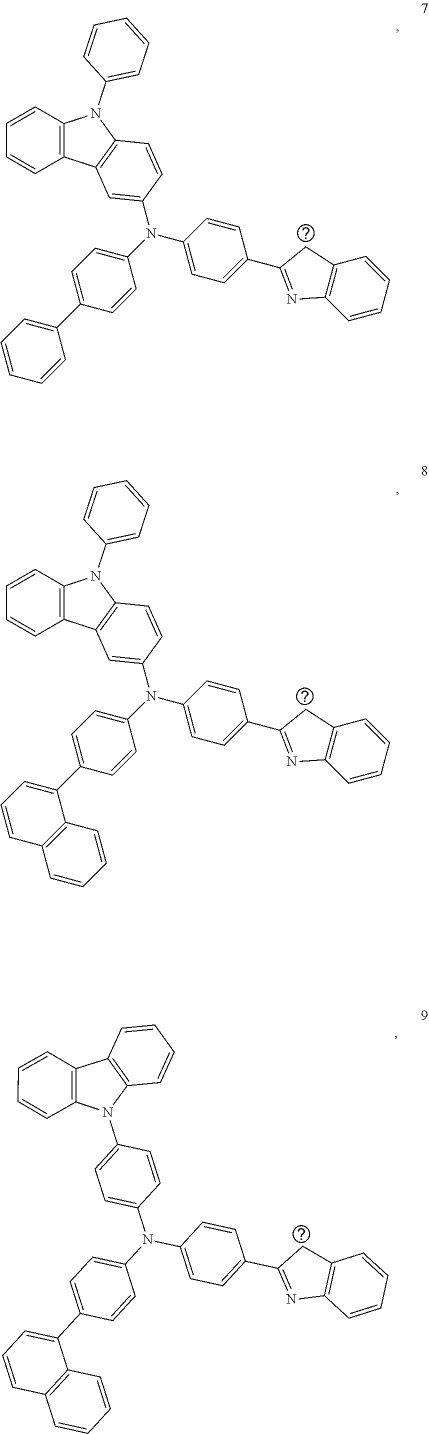 [SMILES: c1ccc(-c2ccc(N(c3ccc(C4=Nc5ccccc5C4)cc3)c3ccc4c(c3)c3ccccc3n4-c3ccccc3)cc2)cc1 ; c1ccc(-n2c3ccccc3c3cc(N(c4ccc(C5=Nc6ccccc6C5)cc4)c4ccc(-c5cccc6ccccc56)cc4)ccc32)cc1 ; c1ccc2c(c1)CC(c1ccc(N(c3ccc(-c4cccc5ccccc45)cc3)c3ccc(-n4c5ccccc5c5ccccc54)cc3)cc1)=N2]

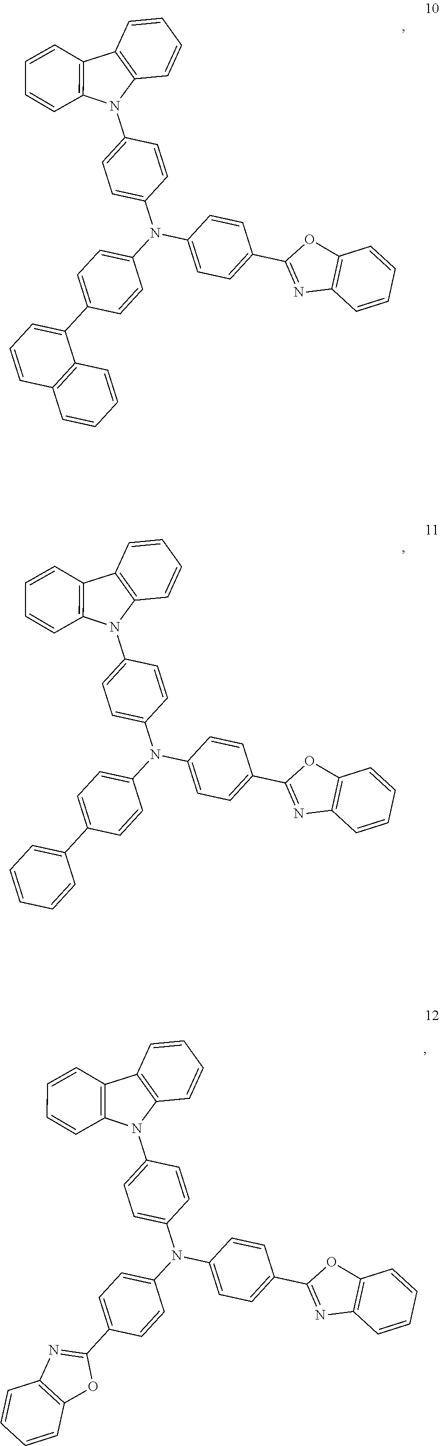 [SMILES: c1ccc2c(-c3ccc(N(c4ccc(-c5nc6ccccc6o5)cc4)c4ccc(-n5c6ccccc6c6ccccc65)cc4)cc3)cccc2c1 ; c1ccc(-c2ccc(N(c3ccc(-c4nc5ccccc5o4)cc3)c3ccc(-n4c5ccccc5c5ccccc54)cc3)cc2)cc1 ; c1ccc2oc(-c3ccc(N(c4ccc(-c5nc6ccccc6o5)cc4)c4ccc(-n5c6ccccc6c6ccccc65)cc4)cc3)nc2c1]

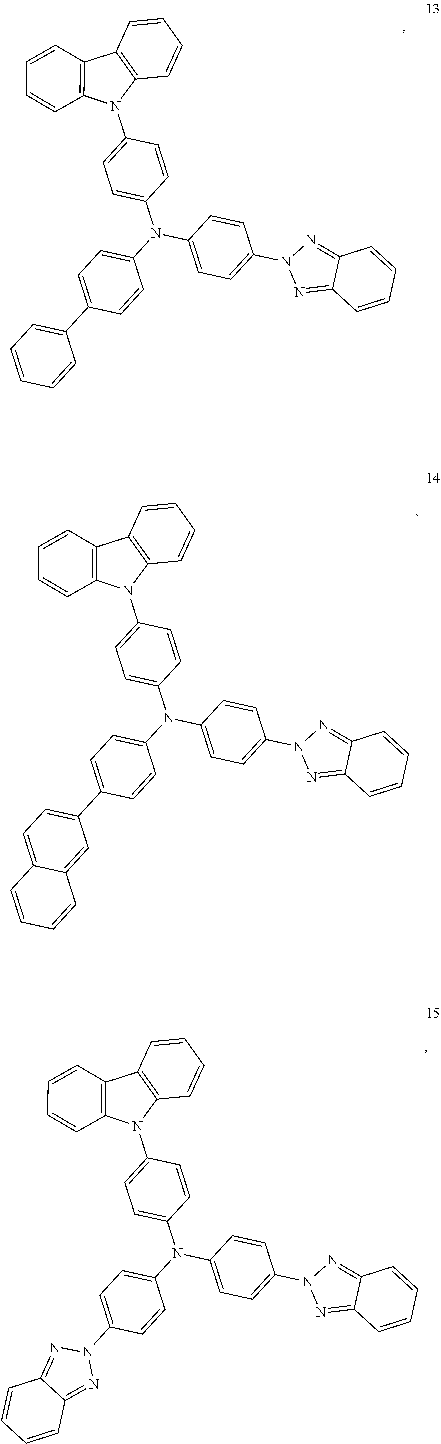 [SMILES: c1ccc(-c2ccc(N(c3ccc(-n4nc5ccccc5n4)cc3)c3ccc(-n4c5ccccc5c5ccccc54)cc3)cc2)cc1 ; c1ccc2cc(-c3ccc(N(c4ccc(-n5nc6ccccc6n5)cc4)c4ccc(-n5c6ccccc6c6ccccc65)cc4)cc3)ccc2c1 ; c1ccc2nn(-c3ccc(N(c4ccc(-n5nc6ccccc6n5)cc4)c4ccc(-n5c6ccccc6c6ccccc65)cc4)cc3)nc2c1]

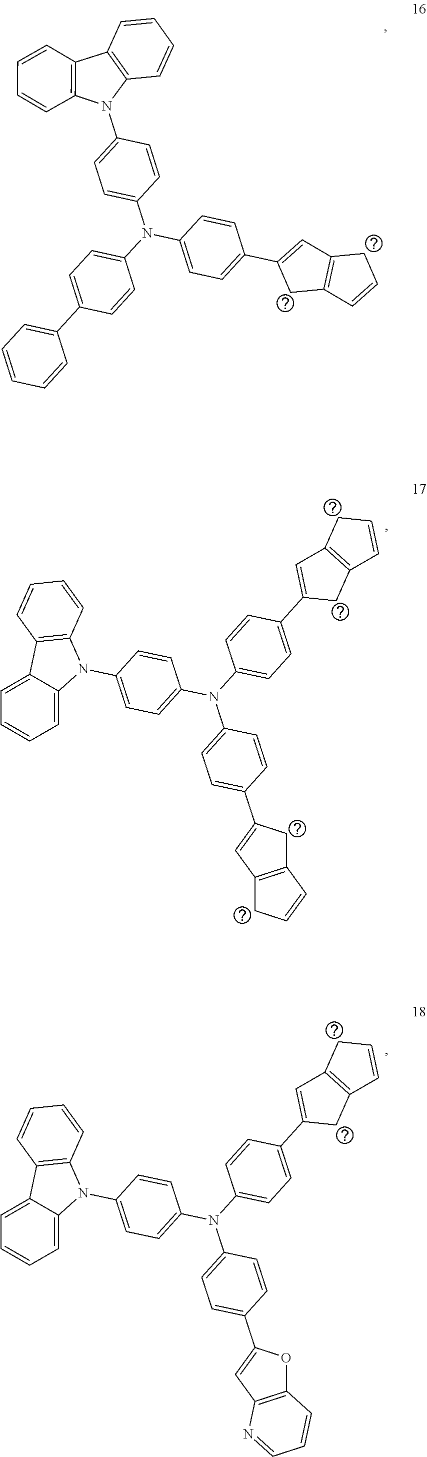 [SMILES: C1=CC2=C(C=C(c3ccc(N(c4ccc(-c5ccccc5)cc4)c4ccc(-n5c6ccccc6c6ccccc65)cc4)cc3)C2)C1 ; C1=CC2=C(C=C(c3ccc(N(c4ccc(C5=CC6=C(C=CC6)C5)cc4)c4ccc(-n5c6ccccc6c6ccccc65)cc4)cc3)C2)C1 ; C1=CC2=C(C=C(c3ccc(N(c4ccc(-c5cc6ncccc6o5)cc4)c4ccc(-n5c6ccccc6c6ccccc65)cc4)cc3)C2)C1]

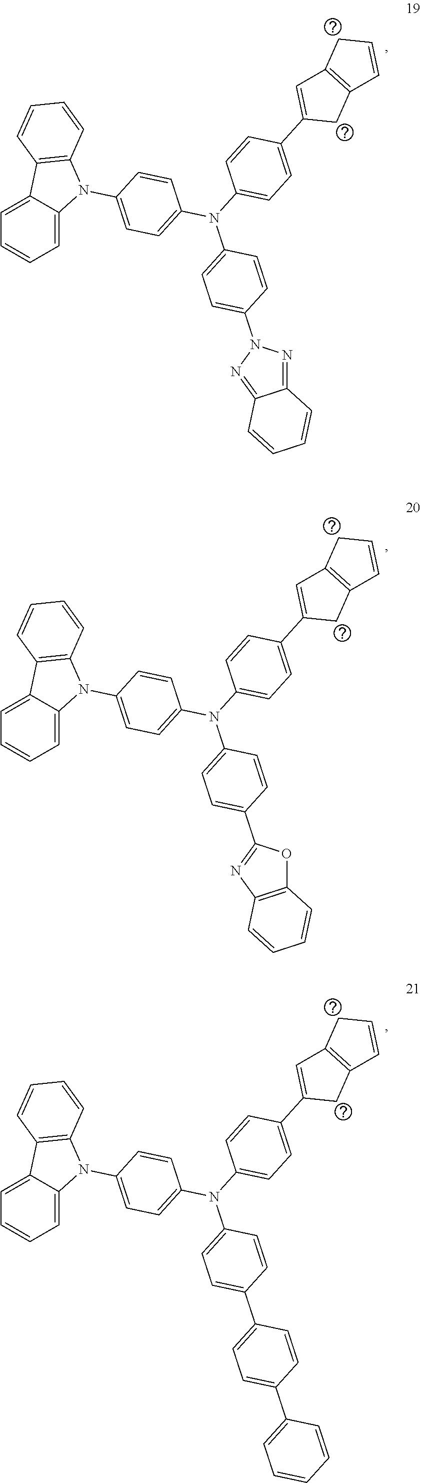 [SMILES: C1=CC2=C(C=C(c3ccc(N(c4ccc(-n5nc6ccccc6n5)cc4)c4ccc(-n5c6ccccc6c6ccccc65)cc4)cc3)C2)C1 ; C1=CC2=C(C=C(c3ccc(N(c4ccc(-c5nc6ccccc6o5)cc4)c4ccc(-n5c6ccccc6c6ccccc65)cc4)cc3)C2)C1 ; C1=CC2=C(C=C(c3ccc(N(c4ccc(-c5ccc(-c6ccccc6)cc5)cc4)c4ccc(-n5c6ccccc6c6ccccc65)cc4)cc3)C2)C1]

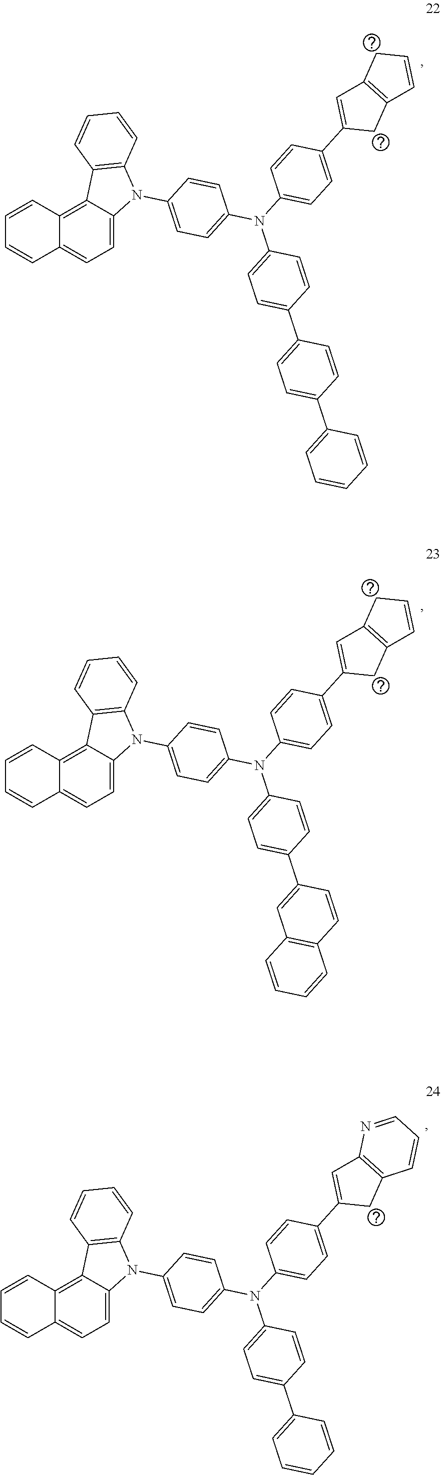 [SMILES: C1=CC2=C(C=C(c3ccc(N(c4ccc(-c5ccc(-c6ccccc6)cc5)cc4)c4ccc(-n5c6ccccc6c6c7ccccc7ccc65)cc4)cc3)C2)C1 ; C1=CC2=C(C=C(c3ccc(N(c4ccc(-c5ccc6ccccc6c5)cc4)c4ccc(-n5c6ccccc6c6c7ccccc7ccc65)cc4)cc3)C2)C1 ; C1=C(c2ccc(N(c3ccc(-c4ccccc4)cc3)c3ccc(-n4c5ccccc5c5c6ccccc6ccc54)cc3)cc2)Cc2cccnc21]

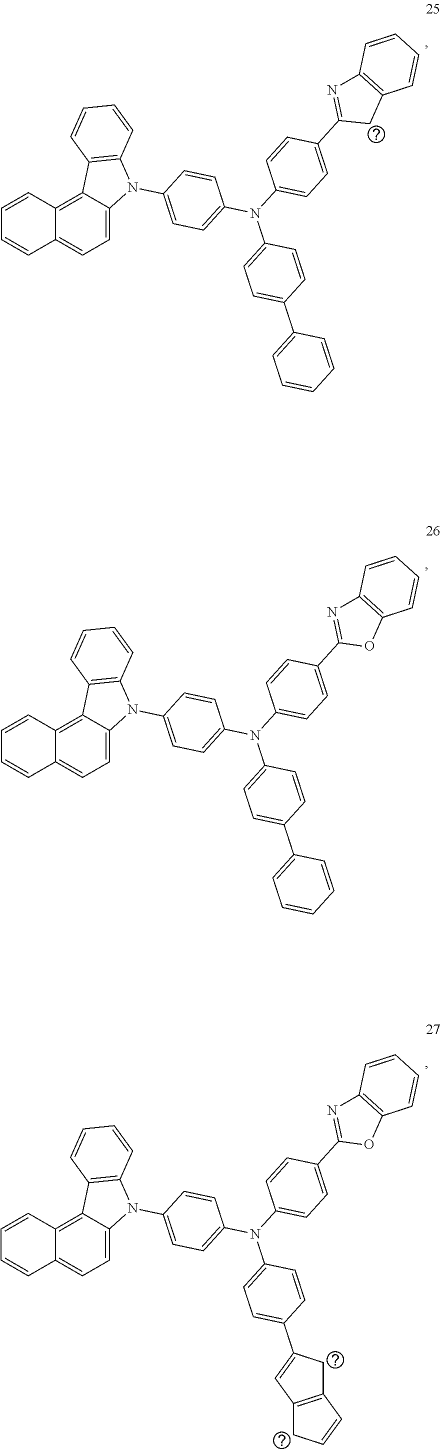 [SMILES: c1ccc(-c2ccc(N(c3ccc(C4=Nc5ccccc5C4)cc3)c3ccc(-n4c5ccccc5c5c6ccccc6ccc54)cc3)cc2)cc1 ; c1ccc(-c2ccc(N(c3ccc(-c4nc5ccccc5o4)cc3)c3ccc(-n4c5ccccc5c5c6ccccc6ccc54)cc3)cc2)cc1 ; C1=CC2=C(C=C(c3ccc(N(c4ccc(-c5nc6ccccc6o5)cc4)c4ccc(-n5c6ccccc6c6c7ccccc7ccc65)cc4)cc3)C2)C1]

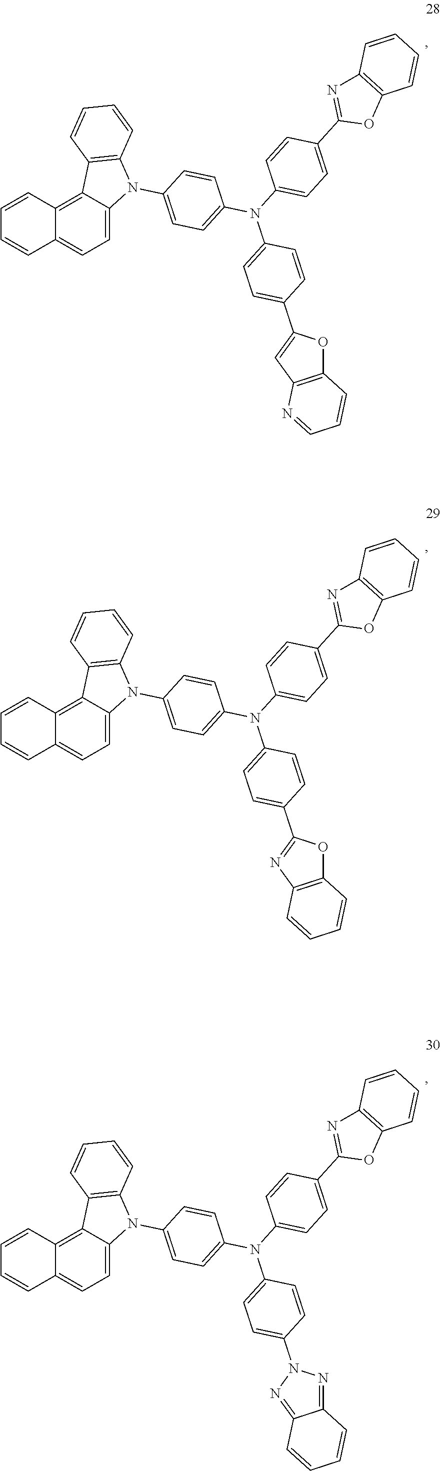 [SMILES: c1ccc2c(c1)ccc1c2c2ccccc2n1-c1ccc(N(c2ccc(-c3cc4ncccc4o3)cc2)c2ccc(-c3nc4ccccc4o3)cc2)cc1 ; c1ccc2c(c1)ccc1c2c2ccccc2n1-c1ccc(N(c2ccc(-c3nc4ccccc4o3)cc2)c2ccc(-c3nc4ccccc4o3)cc2)cc1 ; c1ccc2c(c1)ccc1c2c2ccccc2n1-c1ccc(N(c2ccc(-c3nc4ccccc4o3)cc2)c2ccc(-n3nc4ccccc4n3)cc2)cc1]

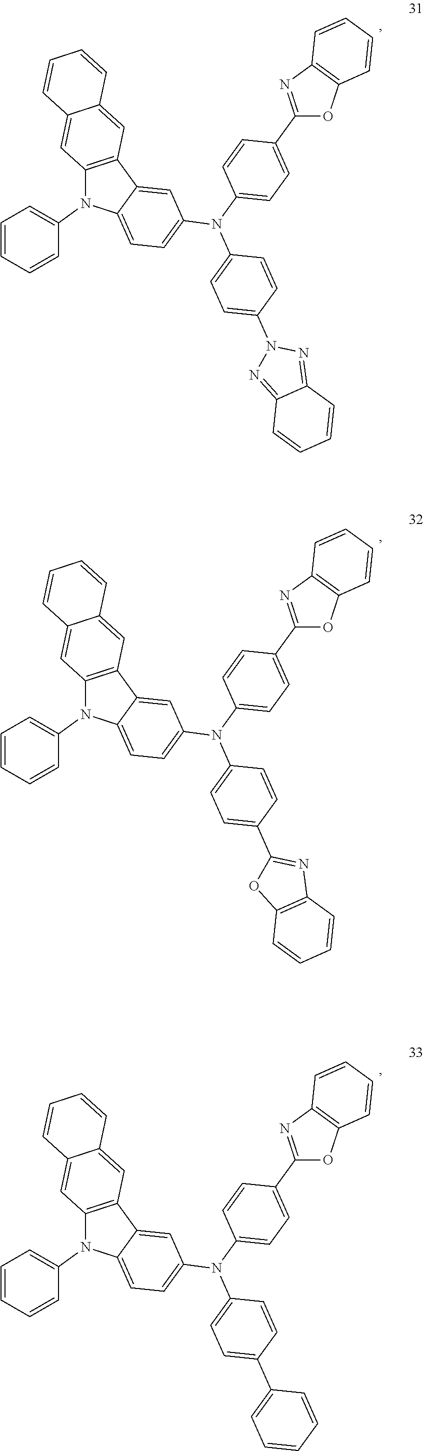 [SMILES: c1ccc(-n2c3ccc(N(c4ccc(-c5nc6ccccc6o5)cc4)c4ccc(-n5nc6ccccc6n5)cc4)cc3c3cc4ccccc4cc32)cc1 ; c1ccc(-n2c3ccc(N(c4ccc(-c5nc6ccccc6o5)cc4)c4ccc(-c5nc6ccccc6o5)cc4)cc3c3cc4ccccc4cc32)cc1 ; c1ccc(-c2ccc(N(c3ccc(-c4nc5ccccc5o4)cc3)c3ccc4c(c3)c3cc5ccccc5cc3n4-c3ccccc3)cc2)cc1]

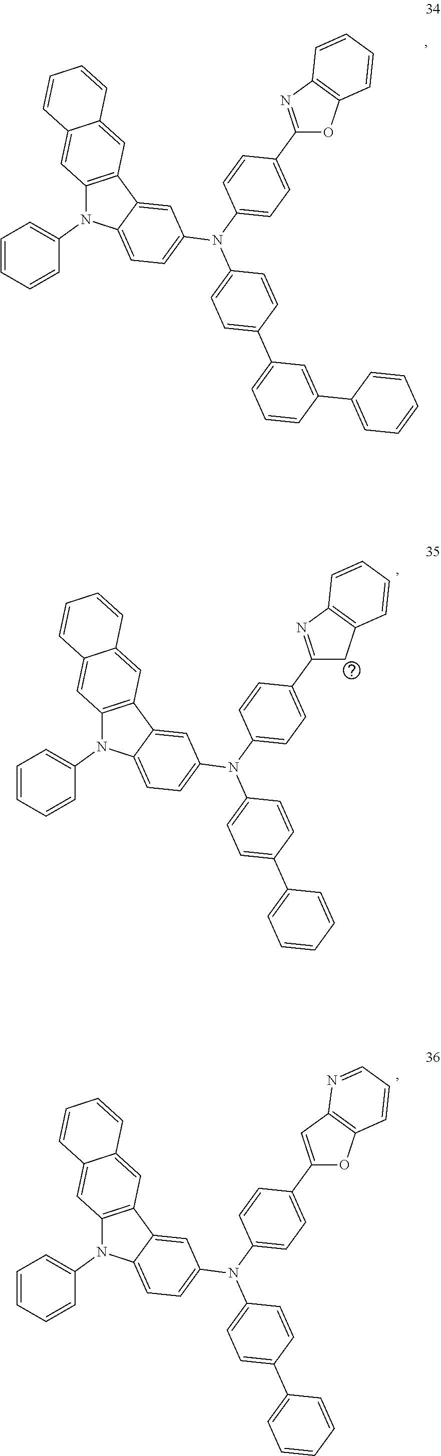 [SMILES: c1ccc(-c2cccc(-c3ccc(N(c4ccc(-c5nc6ccccc6o5)cc4)c4ccc5c(c4)c4cc6ccccc6cc4n5-c4ccccc4)cc3)c2)cc1 ; c1ccc(-c2ccc(N(c3ccc(C4=Nc5ccccc5C4)cc3)c3ccc4c(c3)c3cc5ccccc5cc3n4-c3ccccc3)cc2)cc1 ; c1ccc(-c2ccc(N(c3ccc(-c4cc5ncccc5o4)cc3)c3ccc4c(c3)c3cc5ccccc5cc3n4-c3ccccc3)cc2)cc1]

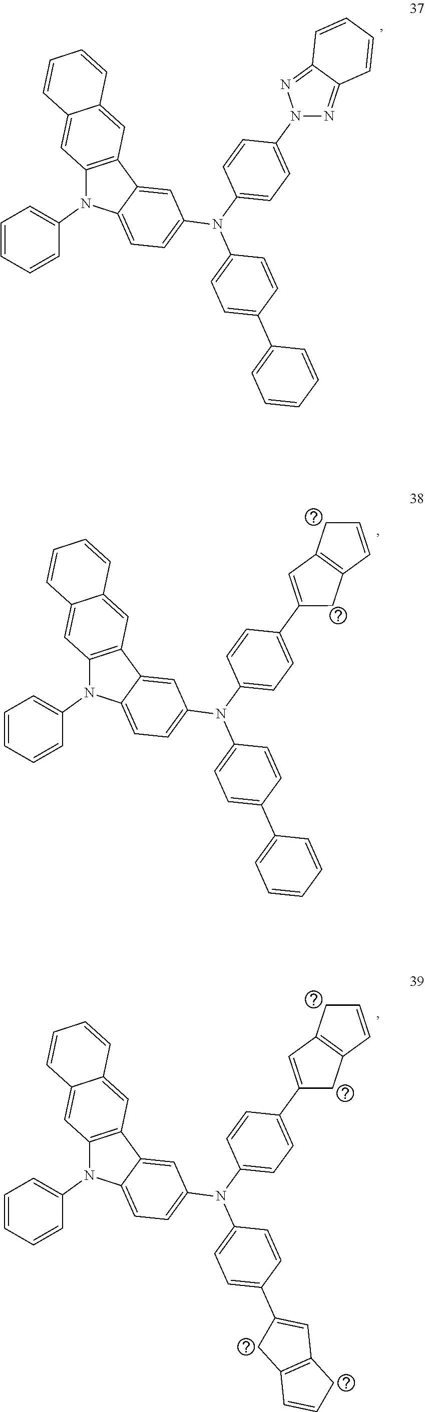 [SMILES: c1ccc(-c2ccc(N(c3ccc(-n4nc5ccccc5n4)cc3)c3ccc4c(c3)c3cc5ccccc5cc3n4-c3ccccc3)cc2)cc1 ; C1=CC2=C(C=C(c3ccc(N(c4ccc(-c5ccccc5)cc4)c4ccc5c(c4)c4cc6ccccc6cc4n5-c4ccccc4)cc3)C2)C1 ; C1=CC2=C(C=C(c3ccc(N(c4ccc(C5=CC6=C(C=CC6)C5)cc4)c4ccc5c(c4)c4cc6ccccc6cc4n5-c4ccccc4)cc3)C2)C1]

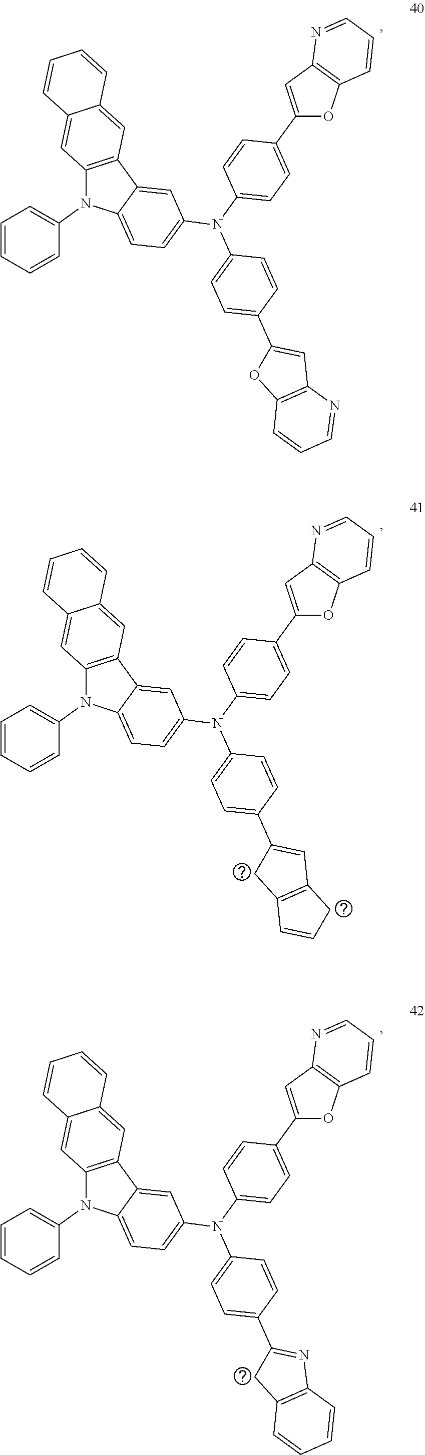 [SMILES: c1ccc(-n2c3ccc(N(c4ccc(-c5cc6ncccc6o5)cc4)c4ccc(-c5cc6ncccc6o5)cc4)cc3c3cc4ccccc4cc32)cc1 ; C1=CC2=C(C=C(c3ccc(N(c4ccc(-c5cc6ncccc6o5)cc4)c4ccc5c(c4)c4cc6ccccc6cc4n5-c4ccccc4)cc3)C2)C1 ; c1ccc(-n2c3ccc(N(c4ccc(C5=Nc6ccccc6C5)cc4)c4ccc(-c5cc6ncccc6o5)cc4)cc3c3cc4ccccc4cc32)cc1]

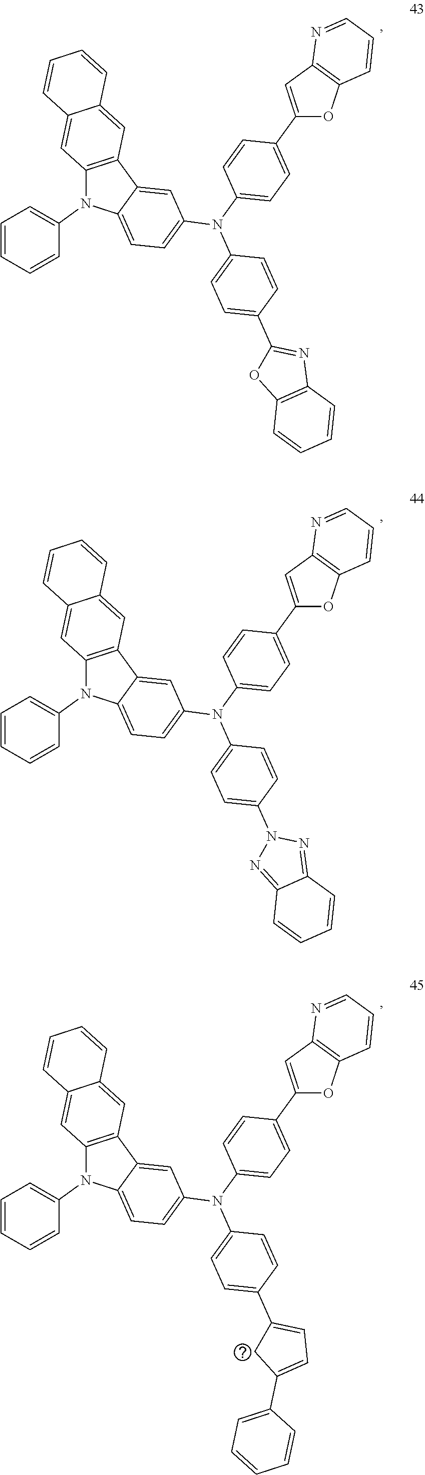 [SMILES: c1ccc(-n2c3ccc(N(c4ccc(-c5cc6ncccc6o5)cc4)c4ccc(-c5nc6ccccc6o5)cc4)cc3c3cc4ccccc4cc32)cc1 ; c1ccc(-n2c3ccc(N(c4ccc(-c5cc6ncccc6o5)cc4)c4ccc(-n5nc6ccccc6n5)cc4)cc3c3cc4ccccc4cc32)cc1 ; C1=C(c2ccccc2)CC(c2ccc(N(c3ccc(-c4cc5ncccc5o4)cc3)c3ccc4c(c3)c3cc5ccccc5cc3n4-c3ccccc3)cc2)=C1]

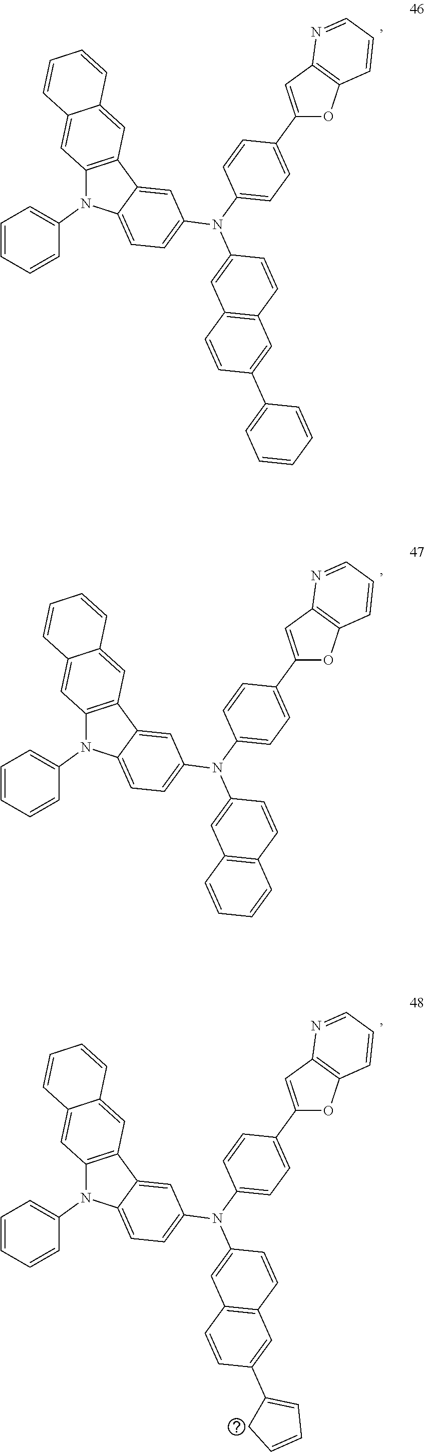 [SMILES: c1ccc(-c2ccc3cc(N(c4ccc(-c5cc6ncccc6o5)cc4)c4ccc5c(c4)c4cc6ccccc6cc4n5-c4ccccc4)ccc3c2)cc1 ; c1ccc(-n2c3ccc(N(c4ccc(-c5cc6ncccc6o5)cc4)c4ccc5ccccc5c4)cc3c3cc4ccccc4cc32)cc1 ; C1=CCC(c2ccc3cc(N(c4ccc(-c5cc6ncccc6o5)cc4)c4ccc5c(c4)c4cc6ccccc6cc4n5-c4ccccc4)ccc3c2)=C1]

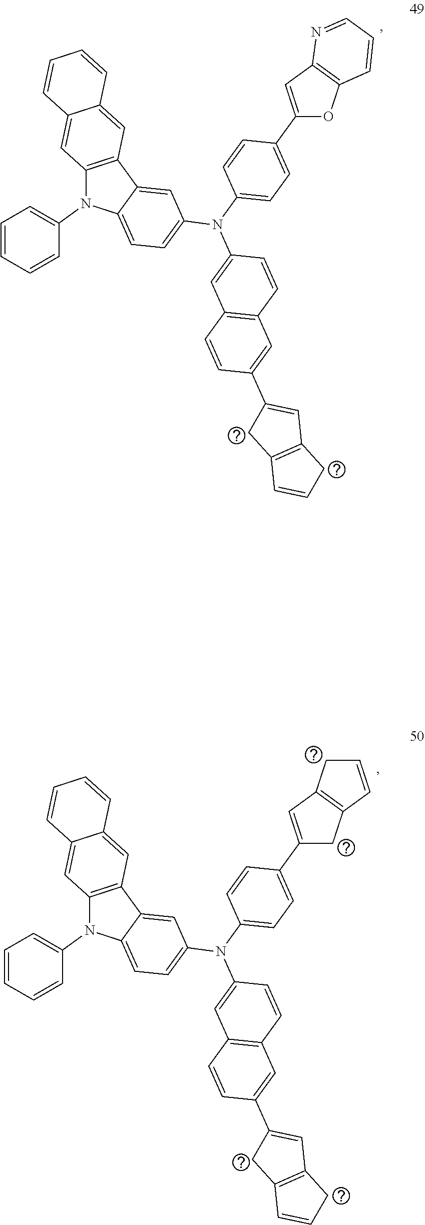 [SMILES: C1=CC2=C(C=C(c3ccc4cc(N(c5ccc(-c6cc7ncccc7o6)cc5)c5ccc6c(c5)c5cc7ccccc7cc5n6-c5ccccc5)ccc4c3)C2)C1 ; C1=CC2=C(C=C(c3ccc(N(c4ccc5cc(C6=CC7=C(C=CC7)C6)ccc5c4)c4ccc5c(c4)c4cc6ccccc6cc4n5-c4ccccc4)cc3)C2)C1]

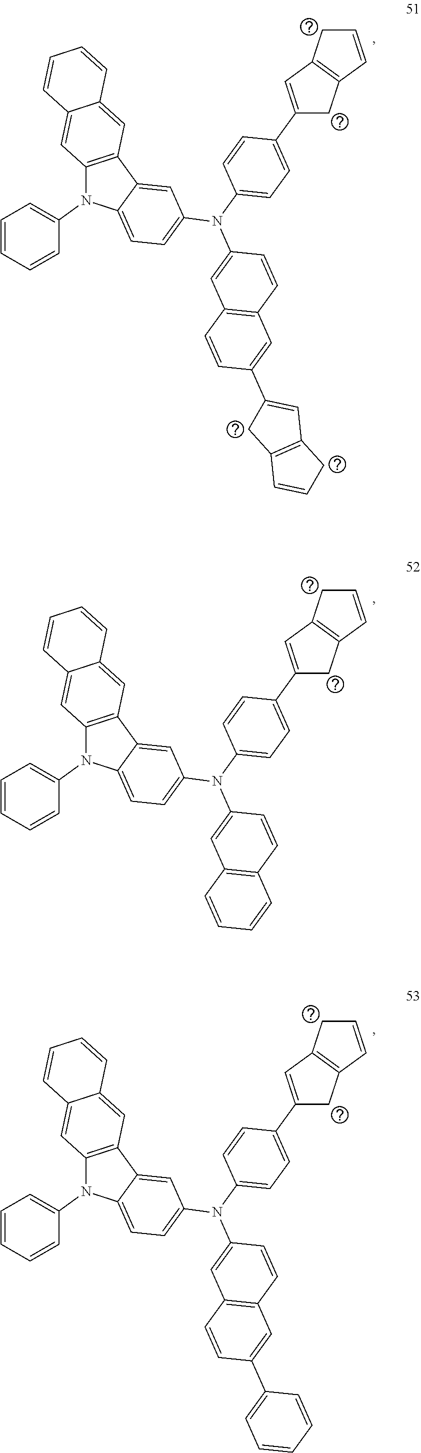 [SMILES: C1=CC2=C(C=C(c3ccc(N(c4ccc5cc(C6=CC7=C(C=CC7)C6)ccc5c4)c4ccc5c(c4)c4cc6ccccc6cc4n5-c4ccccc4)cc3)C2)C1 ; C1=CC2=C(C=C(c3ccc(N(c4ccc5ccccc5c4)c4ccc5c(c4)c4cc6ccccc6cc4n5-c4ccccc4)cc3)C2)C1 ; C1=CC2=C(C=C(c3ccc(N(c4ccc5cc(-c6ccccc6)ccc5c4)c4ccc5c(c4)c4cc6ccccc6cc4n5-c4ccccc4)cc3)C2)C1]

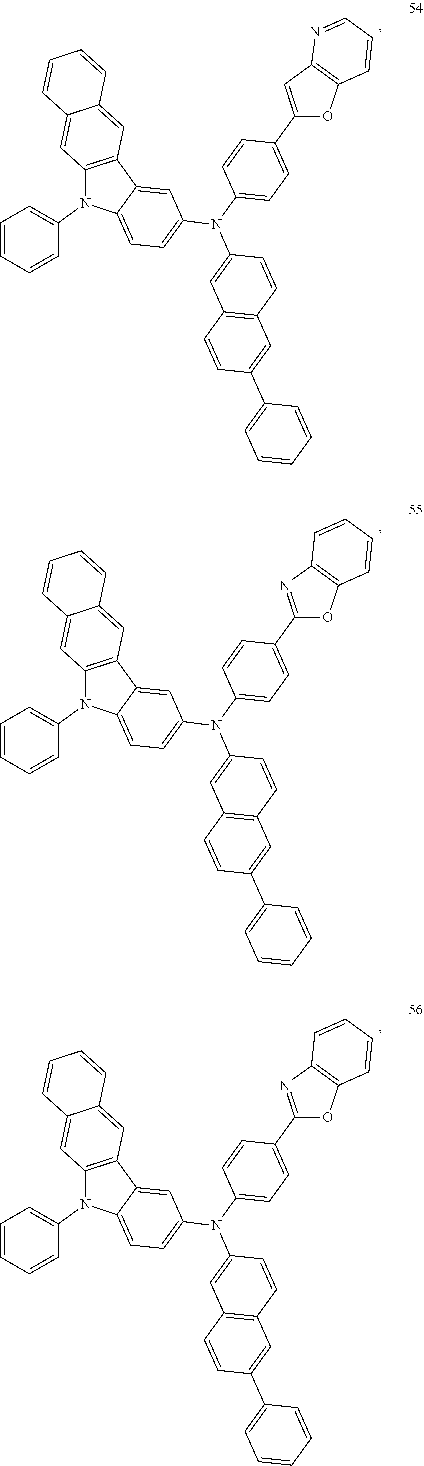 [SMILES: c1ccc(-c2ccc3cc(N(c4ccc(-c5cc6ncccc6o5)cc4)c4ccc5c(c4)c4cc6ccccc6cc4n5-c4ccccc4)ccc3c2)cc1 ; c1ccc(-c2ccc3cc(N(c4ccc(-c5nc6ccccc6o5)cc4)c4ccc5c(c4)c4cc6ccccc6cc4n5-c4ccccc4)ccc3c2)cc1 ; c1ccc(-c2ccc3cc(N(c4ccc(-c5nc6ccccc6o5)cc4)c4ccc5c(c4)c4cc6ccccc6cc4n5-c4ccccc4)ccc3c2)cc1]

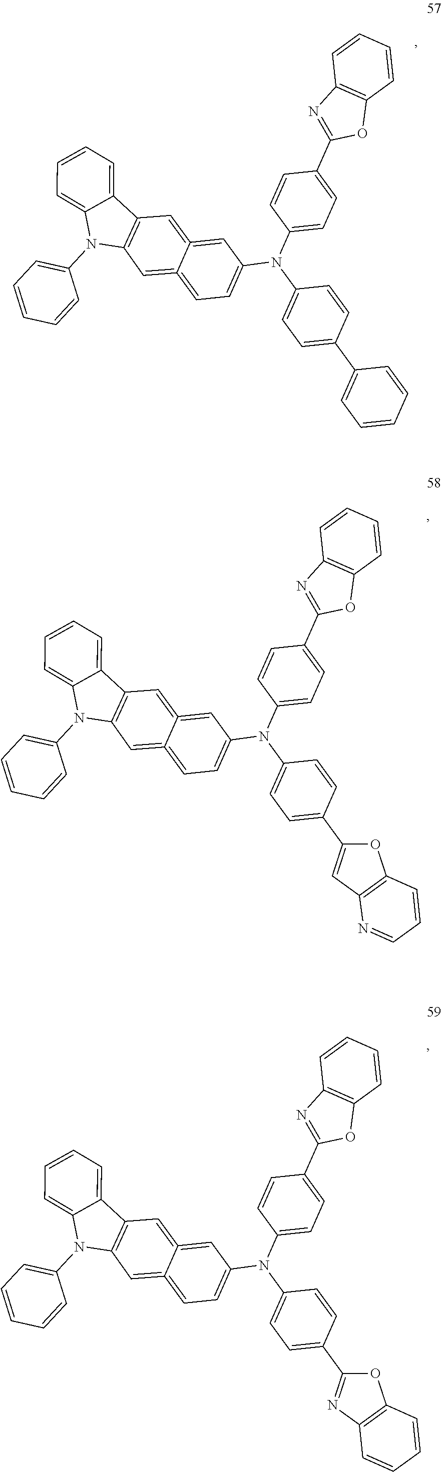

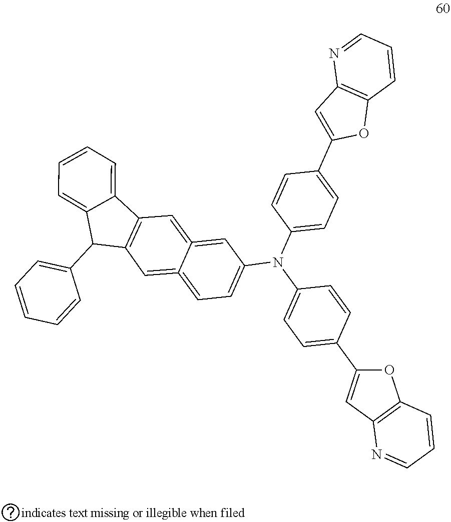

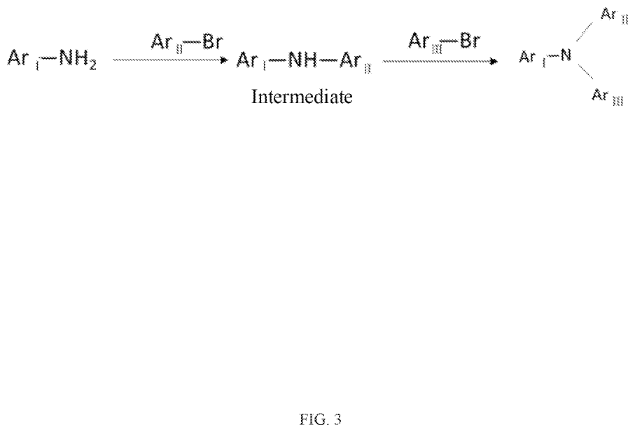

An embodiment of the application further provides a method for preparing a compound of aromatic amines including a group of a derivative of carbazoles. Referring to that shown in FIG. 3. The method includes:

Step 1: Synthetizing an intermediate.

Adding 400 ml of toluene solvent into a reaction flask, and then respectively adding 2 g of Ar₁-NH₂, 2 g of Arn-Br and 1 g of sodium tert-butanol. After filling nitrogen gas, adding 0.3 g of palladium acetate. Then refilling nitrogen gas, and adding 100 ml of toluene solution of 0.3 mol of tert-butylphosphine. After repeating filling nitrogen gas, performing a reflux reaction for 2 hours. After the reaction is completed, the mixture is cooled to a room temperature, and is filtered by diatomite to obtain a filtrate. After the filtrate is concentrated, methanol is added in the filtrate, then the filtrate is stood to recrystallize, and then recrystallization solid is obtained by filtering and by washing with methanol, that is, the intermediate is obtained.

Step 2: Synthetizing the compound of aromatic amines including the group of the derivative of carbazoles.

Adding 400 ml of toluene solvent into the reaction flask, and then successively adding 4 g of raw material of the intermediate, 2 g of Arm-Br, and 1 g of sodium tert-butanol. After filling nitrogen gas, adding palladium acetate. Then refilling nitrogen gas, and adding 100 ml of toluene solution of 0.3 mol of tert-butylphosphine. After repeating filling nitrogen gas, performing a reflux reaction for 2 hours. After the reaction is completed, the mixture is cooled to a room temperature, and is filtered by diatomite to obtain a filtrate. After the filtrate is concentrated, then heated. Adding a small amount of ethanol into the filtrate, then the filtrate is stood to recrystallize, and then recrystallization solid is obtained by filtering and by washing with ethanol, that is, a pale yellow solid compound is obtained.

By using the above synthesis method, the corresponding compounds of aromatic amines may be synthesized by utilizing raw materials in table 1.

TABLE 1
List of raw materials for correspondingly synthesizing a part of the compounds
of aromatic amines provided in the embodiments of the application.

| Ar_{I}-NH₂ | Ar_{II}-Br | Ar_{III}-Br | Compound |
|---|---|---|---|
| 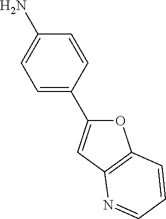 | 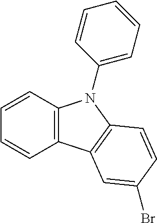 [SMILES: Brc1ccc2c(c1)c1ccccc1n2-c1ccccc1] | 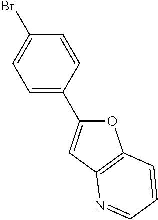 [SMILES: Brc1ccc(-c2cc3ncccc3o2)cc1] | Compound 1 |
| 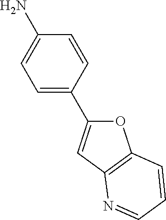 | 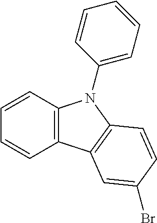 [SMILES: Brc1ccc2c(c1)c1ccccc1n2-c1ccccc1] | 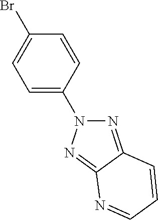 [SMILES: Brc1ccc(-n2nc3cccnc3n2)cc1] | Compound 2 |
| 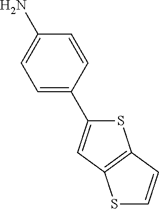 | 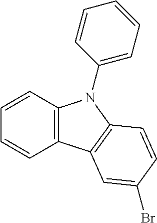 [SMILES: Brc1ccc2c(c1)c1ccccc1n2-c1ccccc1] | 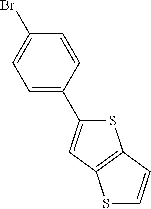 [SMILES: Brc1ccc(-c2cc3sccc3s2)cc1] | Compound 5 |
| 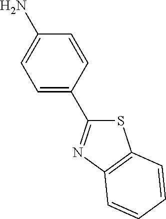 | 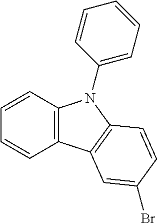 [SMILES: Brc1ccc2c(c1)c1ccccc1n2-c1ccccc1] | 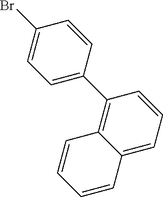 [SMILES: Brc1ccc(-c2cccc3ccccc23)cc1] | Compound 8 |
| 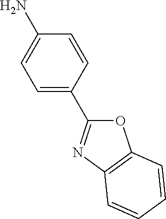 | 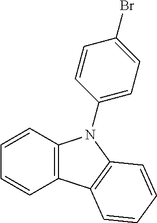 [SMILES: Brc1ccc(-n2c3ccccc3c3ccccc32)cc1] | 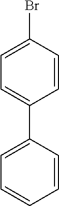 [SMILES: Brc1ccc(-c2ccccc2)cc1] | Compound 11 |
| 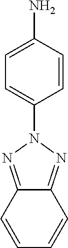 | 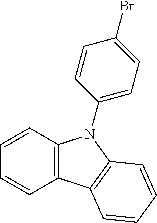 [SMILES: Brc1ccc(-n2c3ccccc3c3ccccc32)cc1] | 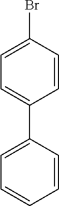 [SMILES: Brc1ccc(-c2ccccc2)cc1] | Compound 13 |
| 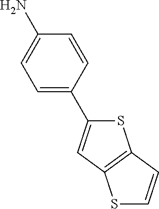 | 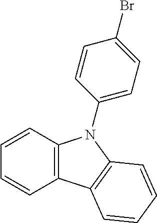 [SMILES: Brc1ccc(-n2c3ccccc3c3ccccc32)cc1] | 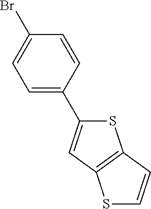 [SMILES: Brc1ccc(-c2cc3sccc3s2)cc1] | Compound 17 |
| 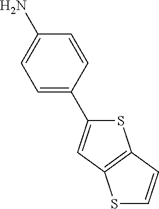 | 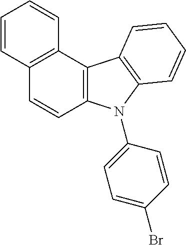 [SMILES: Brc1ccc(-n2c3ccccc3c3c4ccccc4ccc32)cc1] | 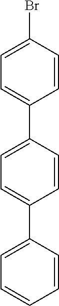 [SMILES: Brc1ccc(-c2ccc(-c3ccccc3)cc2)cc1] | Compound 22 |
| 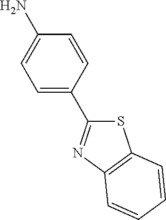 | 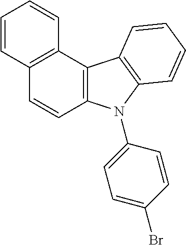 [SMILES: Brc1ccc(-n2c3ccccc3c3c4ccccc4ccc32)cc1] | 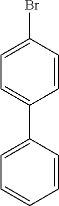 [SMILES: Brc1ccc(-c2ccccc2)cc1] | Compound 25 |
| 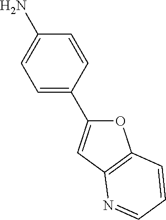 | 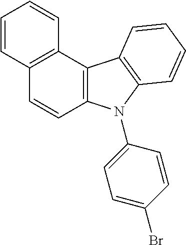 [SMILES: Brc1ccc(-n2c3ccccc3c3c4ccccc4ccc32)cc1] | 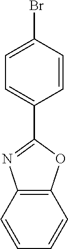 [SMILES: Brc1ccc(-c2nc3ccccc3o2)cc1] | Compound 28 |
| 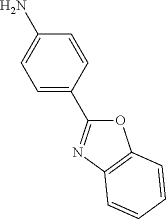 | 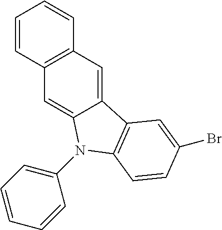 [SMILES: Brc1ccc2c(c1)c1cc3ccccc3cc1n2-c1ccccc1] | 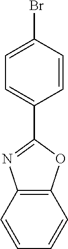 [SMILES: Brc1ccc(-c2nc3ccccc3o2)cc1] | Compound 32 |
| 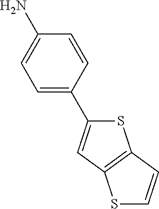 | 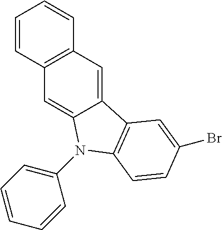 [SMILES: Brc1ccc2c(c1)c1cc3ccccc3cc1n2-c1ccccc1] | 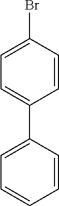 [SMILES: Brc1ccc(-c2ccccc2)cc1] | Compound 38 |
| 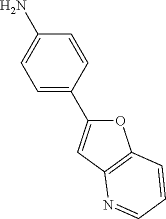 | 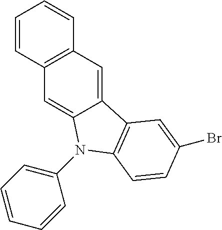 [SMILES: Brc1ccc2c(c1)c1cc3ccccc3cc1n2-c1ccccc1] | 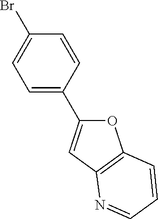 [SMILES: Brc1ccc(-c2cc3ncccc3o2)cc1] | Compound 40 |
| 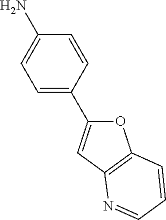 | 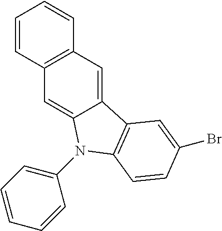 [SMILES: Brc1ccc2c(c1)c1cc3ccccc3cc1n2-c1ccccc1] | 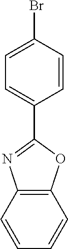 [SMILES: Brc1ccc(-c2nc3ccccc3o2)cc1] | Compound 43 |
| 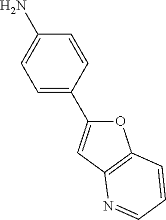 | 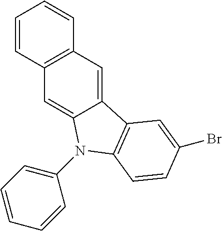 [SMILES: Brc1ccc2c(c1)c1cc3ccccc3cc1n2-c1ccccc1] | 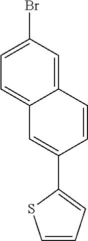 [SMILES: Brc1ccc2cc(-c3cccs3)ccc2c1] | Compound 48 |
| 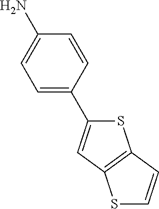 | 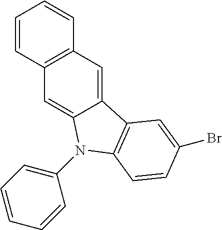 [SMILES: Brc1ccc2c(c1)c1cc3ccccc3cc1n2-c1ccccc1] | 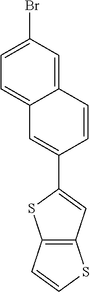 [SMILES: Brc1ccc2cc(-c3cc4sccc4s3)ccc2c1] | Compound 50 |
| 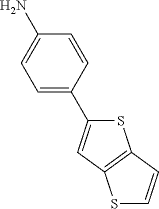 | 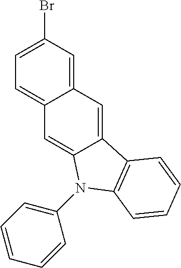 [SMILES: Brc1ccc2cc3c(cc2c1)c1ccccc1n3-c1ccccc1] | 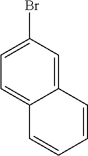 [SMILES: Brc1ccc2ccccc2c1] | Compound 52 |
| 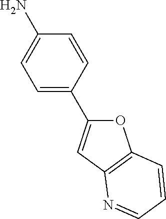 | 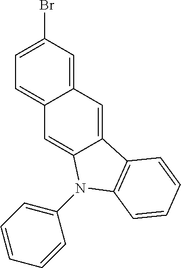 [SMILES: Brc1ccc2cc3c(cc2c1)c1ccccc1n3-c1ccccc1] | 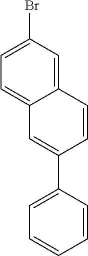 [SMILES: Brc1ccc2cc(-c3ccccc3)ccc2c1] | Compound 54 |
| 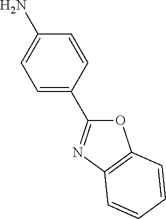 | 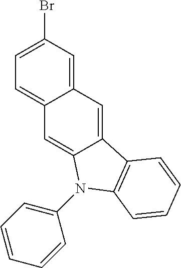 [SMILES: Brc1ccc2cc3c(cc2c1)c1ccccc1n3-c1ccccc1] | 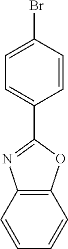 [SMILES: Brc1ccc(-c2nc3ccccc3o2)cc1] | Compound 59 |
| 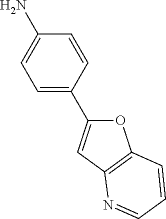 | 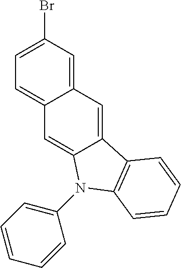 [SMILES: Brc1ccc2cc3c(cc2c1)c1ccccc1n3-c1ccccc1] | 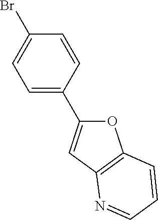 [SMILES: Brc1ccc(-c2cc3ncccc3o2)cc1] | Compound 60 |

In some embodiments of the present application, a glass transition temperature of the compound of aromatic amines ranges from 130° C. to 160° C.

Among them, the glass transition temperature (Tg) determines thermal stability of a material during evaporation. The higher the Tg, the better the thermal stability of the material.

Table 2 provides the glass transition temperatures (Tg) of a part of the compounds of aromatic amines provided by the embodiments of the application, wherein test equipment is a differential scanning calorimeter (DSC), and test conditions are: test atmosphere is nitrogen gas, a heating rate is 10° C. imin, and a test temperature ranges from 50° C. to 300° C.

TABLE 2
Glass transition temperatures of compounds of aromatic amines
in related technology and of a part of the compounds of aromatic
amines provided by the embodiments of the application.

| | Tg ° C. |
|---|---|
| Ref1 (CP1) | 130 |
| Ref2 (CP2) | 125 |
| Compound 1 | 136 |
| Compound 2 | 134 |
| Compound 5 | 135 |
| Compound 8 | 133 |
| Compound 11 | 131 |
| Compound 13 | 132 |
| Compound 17 | 134 |
| Compound 23 | 133 |
| Compound 25 | 134 |
| Compound 28 | 136 |
| Compound 32 | 137 |
| Compound 38 | 136 |
| Compound 40 | 137 |
| Compound 43 | 137 |
| Compound 48 | 135 |
| Compound 50 | 135 |
| Compound 52 | 134 |
| Compound 54 | 134 |
| Compound 59 | 135 |
| Compound 60 | 140 |

Among them, the Ref1 and Ref2 are two light-extraction materials in related technology. According to data in the table 2, compared with glass transition temperatures of the light-extraction materials in the related technology, the compounds of monoarylamines provided by the embodiments of the present application have higher glass transition temperatures. Therefore, in a process of preparing the capping layer by using an evaporation process, the process of preparing the capping layer, in which the material is the compound of monoarylamines provided by the embodiments of the present application, has better process stability.

A structure of the Ref1 (CP1) is

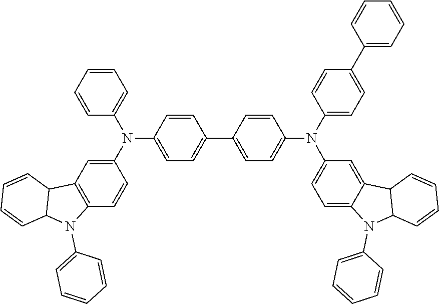 [SMILES: C1=CC2c3cc(N(c4ccccc4)c4ccc(-c5ccc(N(c6ccc(-c7ccccc7)cc6)c6ccc7c(c6)C6C=CC=CC6N7c6ccccc6)cc5)cc4)ccc3N(c3ccccc3)C2C=C1]

A structure of the Ref2 (CP2) is

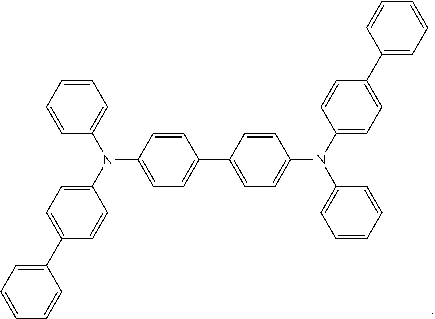 [SMILES: c1ccc(-c2ccc(N(c3ccccc3)c3ccc(-c4ccc(N(c5ccccc5)c5ccc(-c6ccccc6)cc5)cc4)cc3)cc2)cc1]

In some embodiments of the present application, a refractive index of the compound of aromatic amines at a wavelength of 460 nm ranges from 2.0 to 2.2. The refractive index of the compound of aromatic amines at a wavelength of 530 nm ranges from 1.9 to 2.1. The refractive index of the compound of aromatic amines at a wavelength of 620 nm ranges from 1.85 to 2.0. Among them, the refractive index is an important physical parameter of material of the capping layer (CPL), and a size of the refractive index directly determines the light-coupling efficiency of the light-emitting device. Table 3 provides the refractive indexes (n) of a part of the compounds of aromatic amines provided by the embodiments of the application, wherein the test equipment is an ellipsometer, and the test conditions are: a scanning range is from 245 nm to 1000 nm, a film of the compound is evaporated on a silicon chip, and the film has a thickness of 50 nm.

TABLE 3
the refractive indexes of compounds of aromatic amines in
related technology and of a part of the compounds of aromatic
amines provided by the embodiments of the application.

Refractive indexes n of different wavelengths

| Compound | 460 nm | 530 nm | 620 nm |
|---|---|---|---|
| Ref1 (CP1) | 1.93 | 1.86 | 1.83 |
| Ref2 (CP2) | 1.99 | 1.91 | 1.87 |
| Compound 1 | 2.09 | 1.9 [symbol] | 1.94 |
| Compound 2 | 2.0 [symbol] | 1.9 [symbol] | 1.9 |
| Compound 5 | 2.06 | 1.97 | 1.92 |
| Compound 8 | 2.03 | 1.9 [symbol] | 1.89 |
| Compound 11 | 2.01 | 1.92 | 1.89 |
| Compound 13 | 2.02 | 1.9 [symbol] | 1.9 |
| Compound 17 | 2.0 [symbol] | 1.97 | 1.93 |
| Compound 23 | 2.06 | 1.97 | 1.92 |
| Compound 25 | 2.07 | 1.98 | 1.93 |
| Compound 28 | 2.1 | 1.99 | 1.9 [symbol] |
| Compound 32 | 2.11 | 1.98 | 1.9 [symbol] |
| Compound 38 | 2.09 | 1.98 | 1.9 [symbol] |
| Compound 40 | 2.12 | [symbol] | 1.96 |
| Compound 43 | 2.11 | 1.97 | 1.94 |
| Compound 48 | 2.08 | 1.96 | 1.9 [symbol] |
| Compound 50 | 2.07 | 1.98 | 1.9 [symbol] |
| Compound 52 | 2.0 [symbol] | 1.96 | 1.9 [symbol] |
| Compound 54 | 2.06 | 1.9 [symbol] | 1.92 |
| Compound 59 | 2.08 | 1.99 | 1.94 |
| Compound 60 | 2.1 [symbol] | 2.02 | 1.96 |

[symbol]

indicates data missing or illegible when filed

Among them, the Ref1 and the Ref2 are two light-extraction materials in related technology. According to data in the table 3, compared with the refractive indexes of the light-extraction materials in the related technology, the compounds of monoarylamines provided in the embodiments of the present application have higher refractive indexes (including but not limited to circumstances at wavelengths of 460 nm, 530 nm and 620 nm). Therefore, preparing the capping layer by using the compounds of monoarylamines provided in the embodiments of the present application is more conducive to coupling output of light in the light-emitting device, thus improving light-outlet efficiency of the light-emitting device.

In some embodiments of the present application, in a condition that the compound of aromatic amines includes the following structure 1, a general formula of the compound of aromatic amines is a following general formula I,

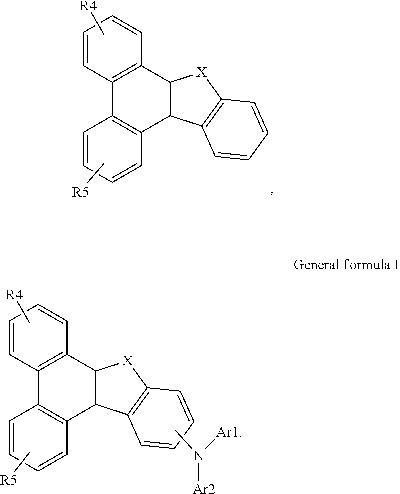

Among them, a structure of at least one of the Ar1 and Ar2 includes one of the following three structures 3,

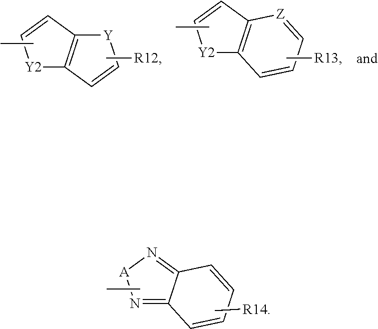

The structure 3 includes at least one heteroatom.

In an exemplary embodiment, the compound of aromatic amines with the general formula

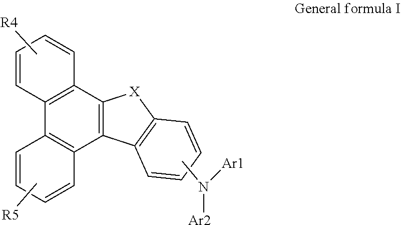

has at least one group of a derivative of phenanthrenes.

In an exemplary embodiment, the Y2 includes O, S or N. The Z includes C or N. The A includes C or N. In an exemplary embodiment, the R12, R13 and R14 respectively include one of: hydrogen, deuterium, halogen, a nitro group, a nitrile group, a substituted or unsubstituted C1 to C30 alkyl group, a substituted or unsubstituted C2 to C30 alkenyl group, a substituted or unsubstituted C1 to C30 alkoxy group, a substituted or unsubstituted C1 to C30 thioether group, a substituted or unsubstituted C6 to C50 aryl group, and a substituted or unsubstituted C2 to C50 heteroaryl group.

In the exemplary embodiment, one of the Ar1 and Ar2 may include one of the following three structures 3, or both the Ar1 and Ar2 may include one of the following three structures 3,

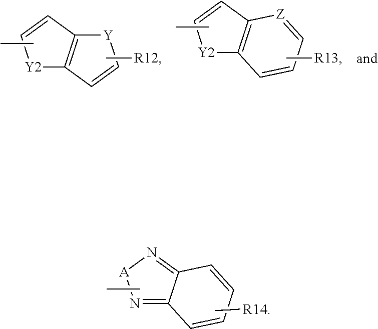

For example, the Ar1 may include

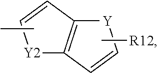

and the Ar2 may include

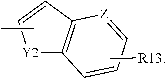

For another example, the Ar1 may include

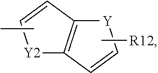

and the Ar2 may include

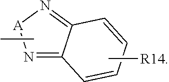

For another example, the Ar1 may include

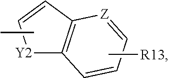

and the Ar2 may include

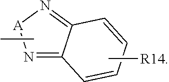

For still another example, both the Ar1 and Ar2 may include

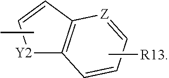

Whether structures of both the Ar1 and the Ar2 are the same is no limited here, which may be determined according to an actual situation.

In some embodiments of the present application, in a condition that the structure of only one of the Ar1 and the Ar2 includes one of

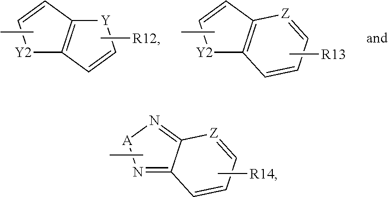

the structure of the other one of the Ar1 and the Ar2 includes any one of: a nitrile group, a C2 to C30 alkyl group, a C3 to C30 naphthyl group, a substituted or unsubstituted C6 to C60 aryl group, and a substituted or substituted C5 to C60 heteroaryl group.

A substituted position in the substituted C6 to C60 aryl group is no limited here, and a specific structure of a substituted group in the substituted C5 to C60 heteroaryl group is no limited here either to, which may be determined according to an actual situation.

For example, the compound of aromatic amines (containing a group a derivative of phenanthrenes) may include any of the following structures:

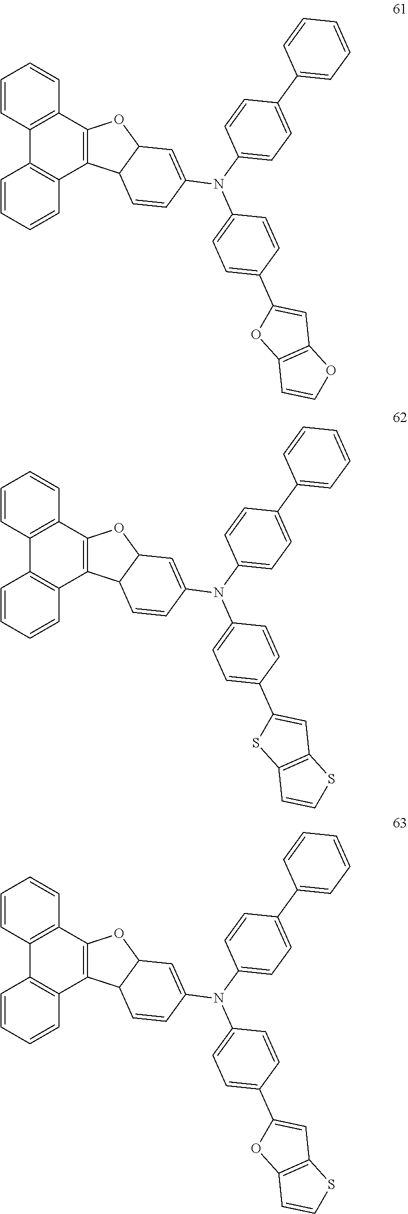

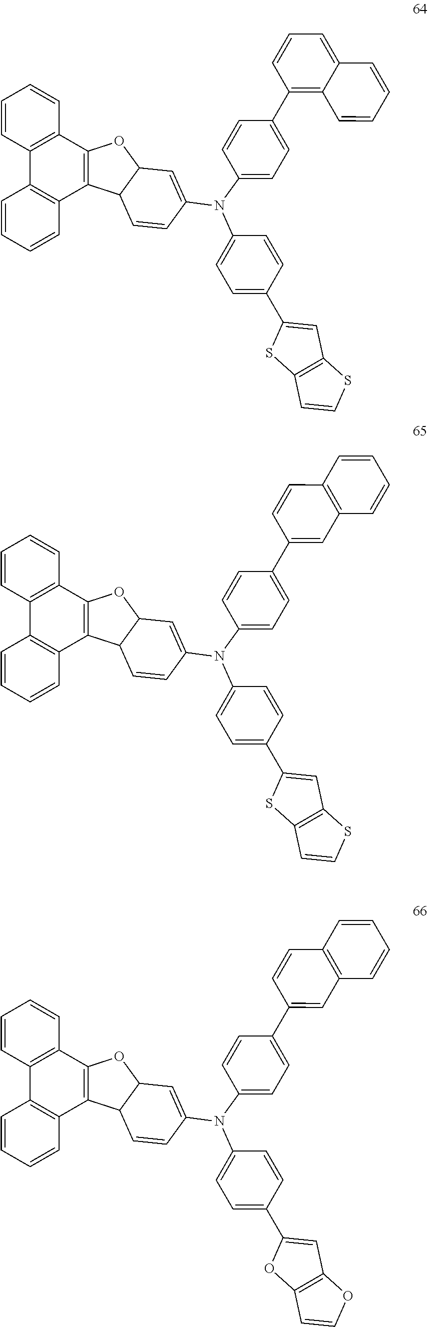

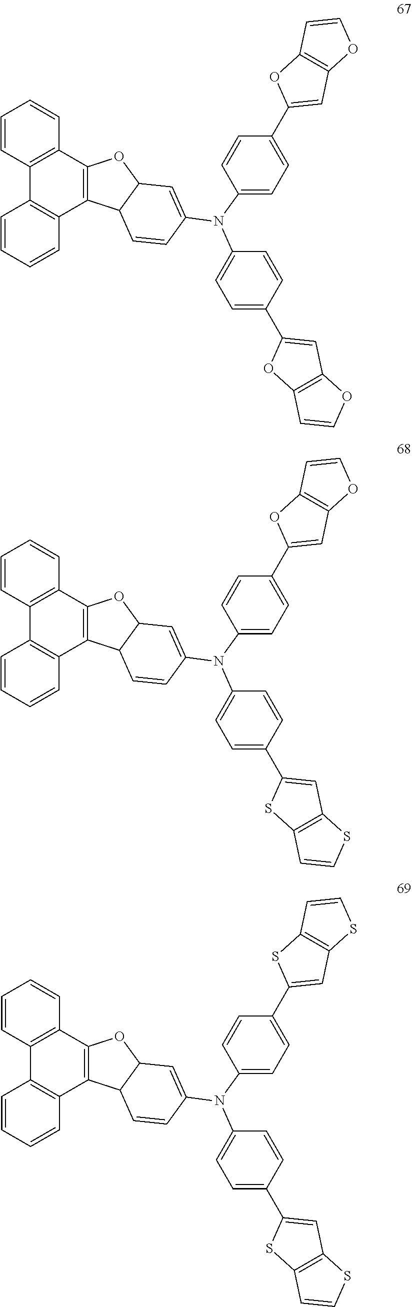

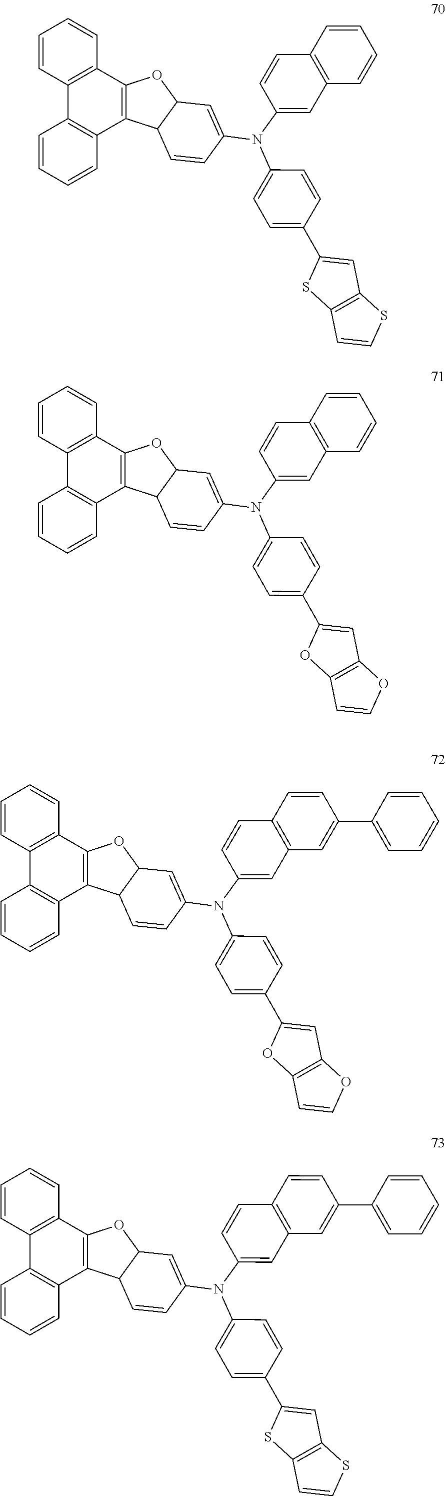 [SMILES: C1=CC2c3c(c4ccccc4c4ccccc34)OC2C=C1N(c1ccc(-c2cc3sccc3s2)cc1)c1ccc2ccccc2c1 ; C1=CC2c3c(c4ccccc4c4ccccc34)OC2C=C1N(c1ccc(-c2cc3occc3o2)cc1)c1ccc2ccccc2c1 ; C1=CC2c3c(c4ccccc4c4ccccc34)OC2C=C1N(c1ccc(-c2cc3occc3o2)cc1)c1ccc2ccc(-c3ccccc3)cc2c1 ; C1=CC2c3c(c4ccccc4c4ccccc34)OC2C=C1N(c1ccc(-c2cc3sccc3s2)cc1)c1ccc2ccc(-c3ccccc3)cc2c1]

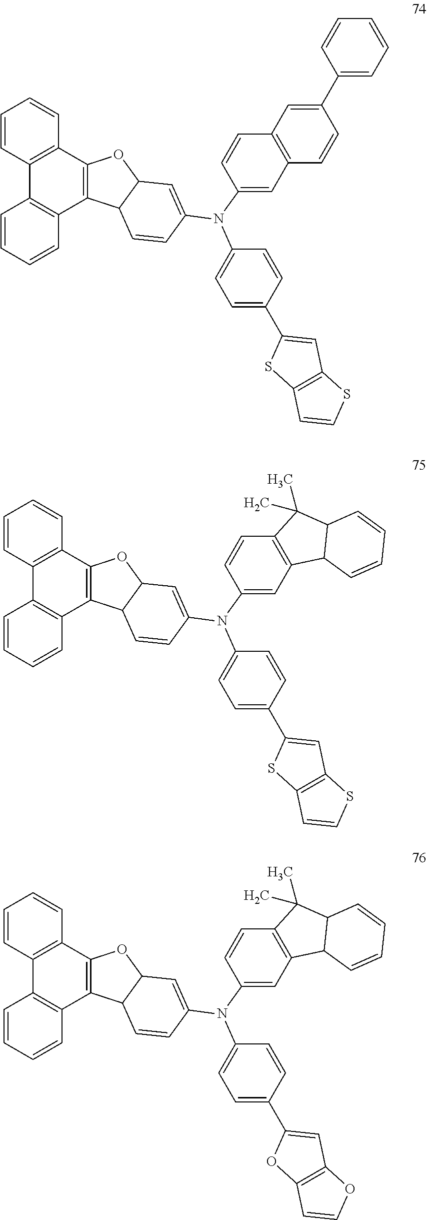 [SMILES: C1=CC2c3c(c4ccccc4c4ccccc34)OC2C=C1N(c1ccc(-c2cc3sccc3s2)cc1)c1ccc2cc(-c3ccccc3)ccc2c1 ; CC1(C)c2ccc(N(C3=CC4Oc5c(c6ccccc6c6ccccc56)C4C=C3)c3ccc(-c4cc5sccc5s4)cc3)cc2C2C=CC=CC21 ; CC1(C)c2ccc(N(C3=CC4Oc5c(c6ccccc6c6ccccc56)C4C=C3)c3ccc(-c4cc5occc5o4)cc3)cc2C2C=CC=CC21]

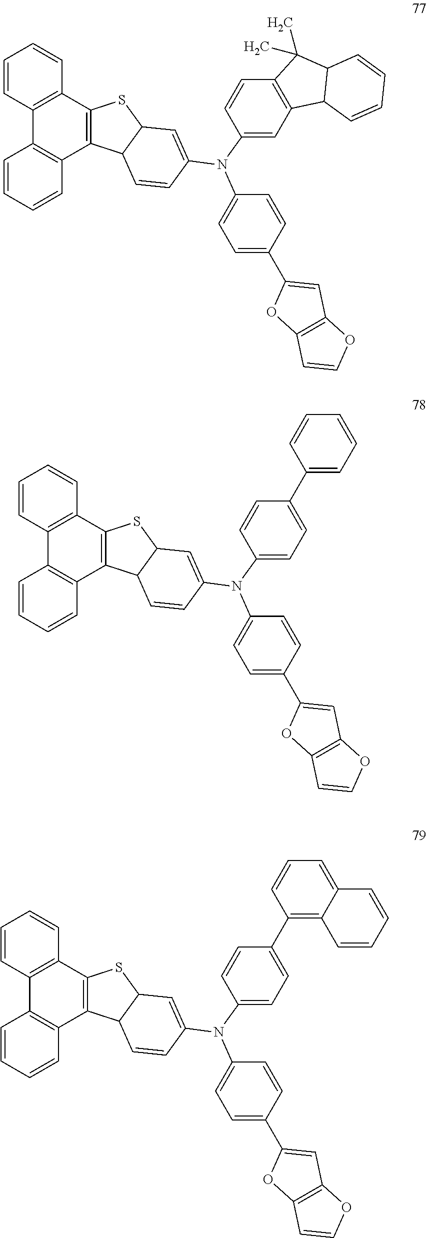

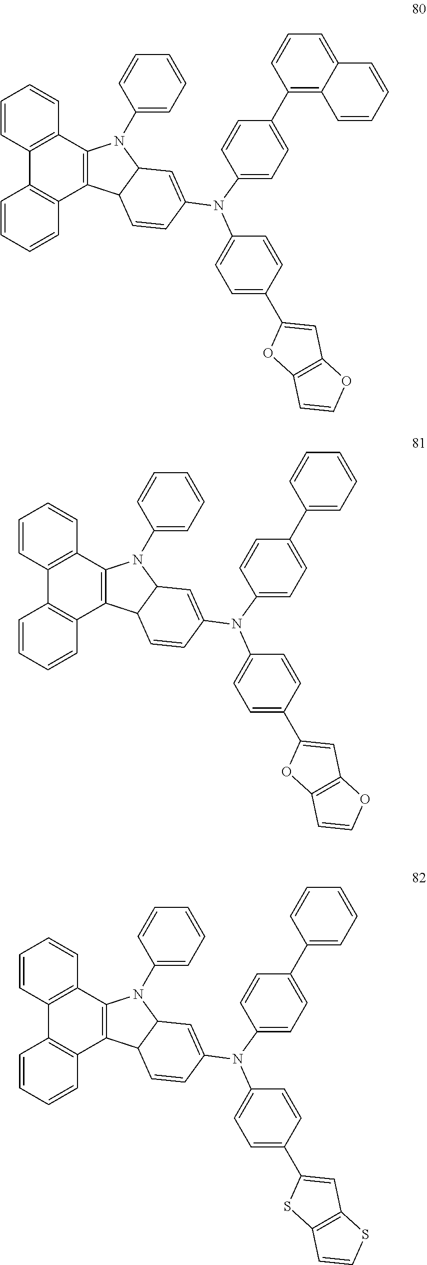

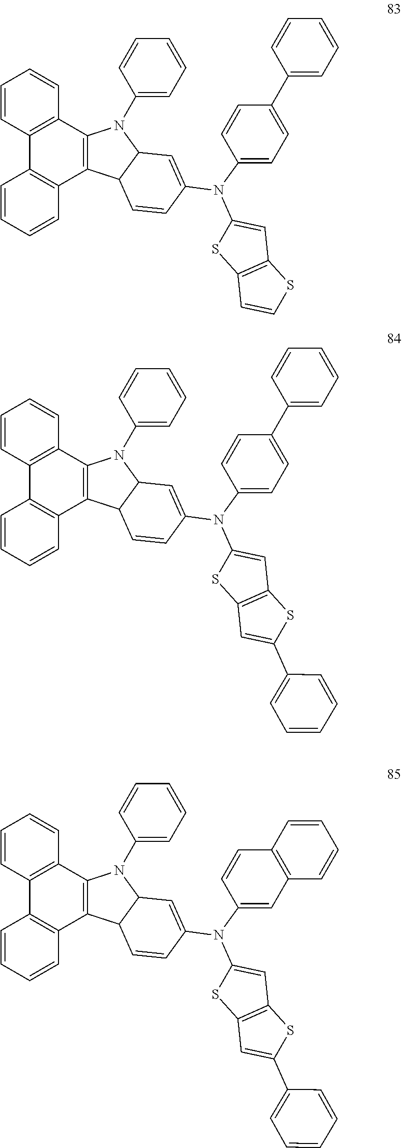

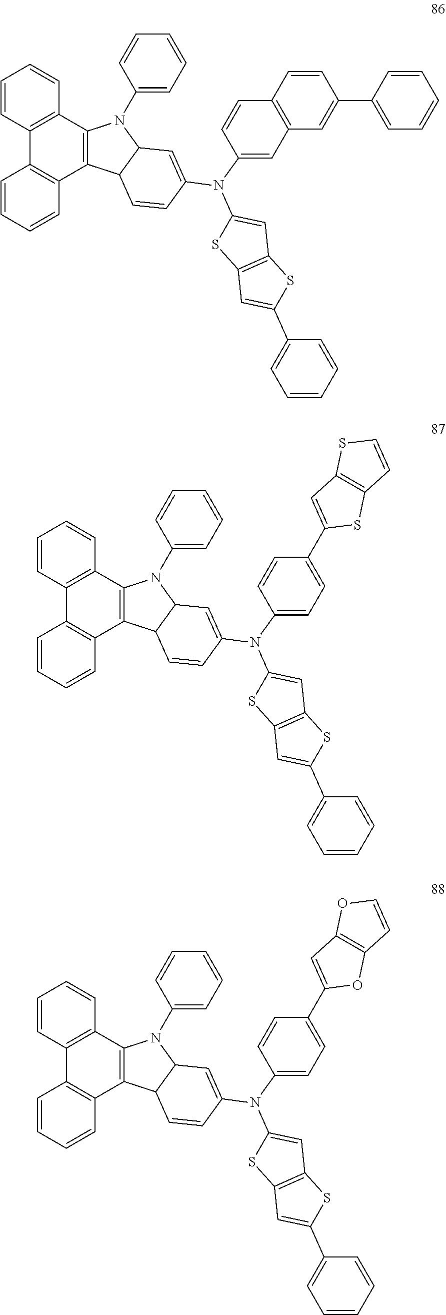 [SMILES: C1=CC2c3c(c4ccccc4c4ccccc34)N(c3ccccc3)C2C=C1N(c1ccc2ccc(-c3ccccc3)cc2c1)c1cc2sc(-c3ccccc3)cc2s1 ; C1=CC2c3c(c4ccccc4c4ccccc34)N(c3ccccc3)C2C=C1N(c1ccc(-c2cc3sccc3s2)cc1)c1cc2sc(-c3ccccc3)cc2s1 ; C1=CC2c3c(c4ccccc4c4ccccc34)N(c3ccccc3)C2C=C1N(c1ccc(-c2cc3occc3o2)cc1)c1cc2sc(-c3ccccc3)cc2s1]

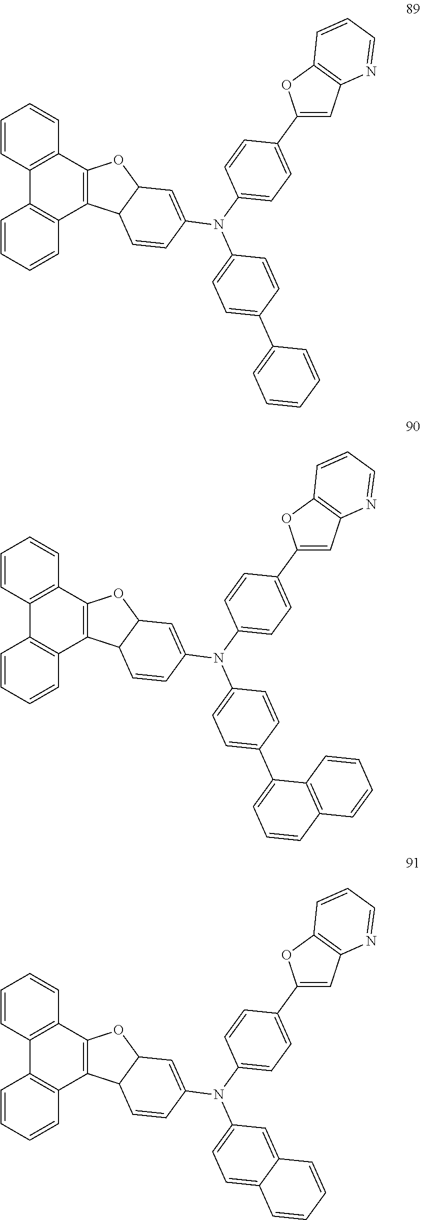 [SMILES: C1=CC2c3c(c4ccccc4c4ccccc34)OC2C=C1N(c1ccc(-c2ccccc2)cc1)c1ccc(-c2cc3ncccc3o2)cc1 ; C1=CC2c3c(c4ccccc4c4ccccc34)OC2C=C1N(c1ccc(-c2cc3ncccc3o2)cc1)c1ccc(-c2cccc3ccccc23)cc1 ; C1=CC2c3c(c4ccccc4c4ccccc34)OC2C=C1N(c1ccc(-c2cc3ncccc3o2)cc1)c1ccc2ccccc2c1]

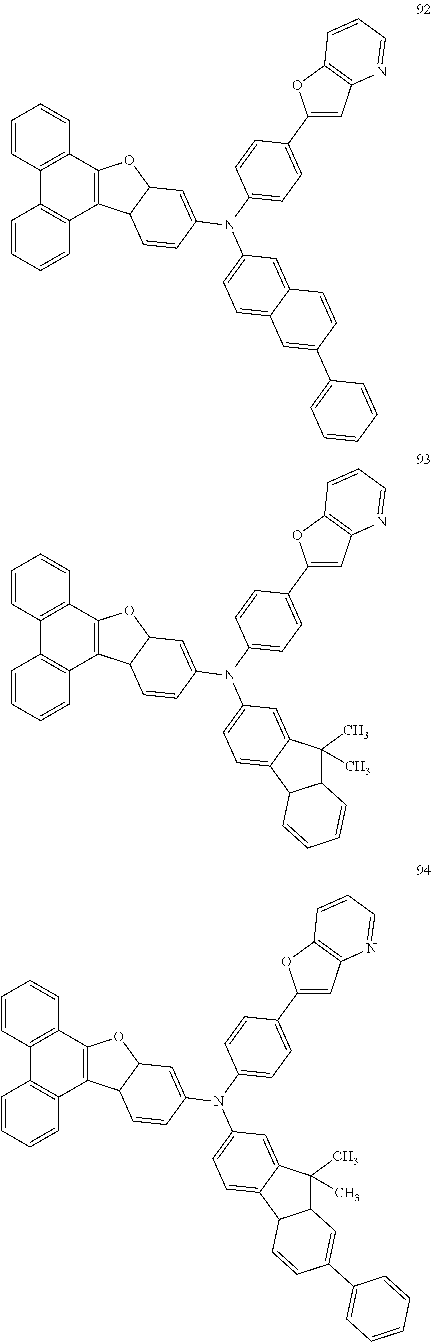

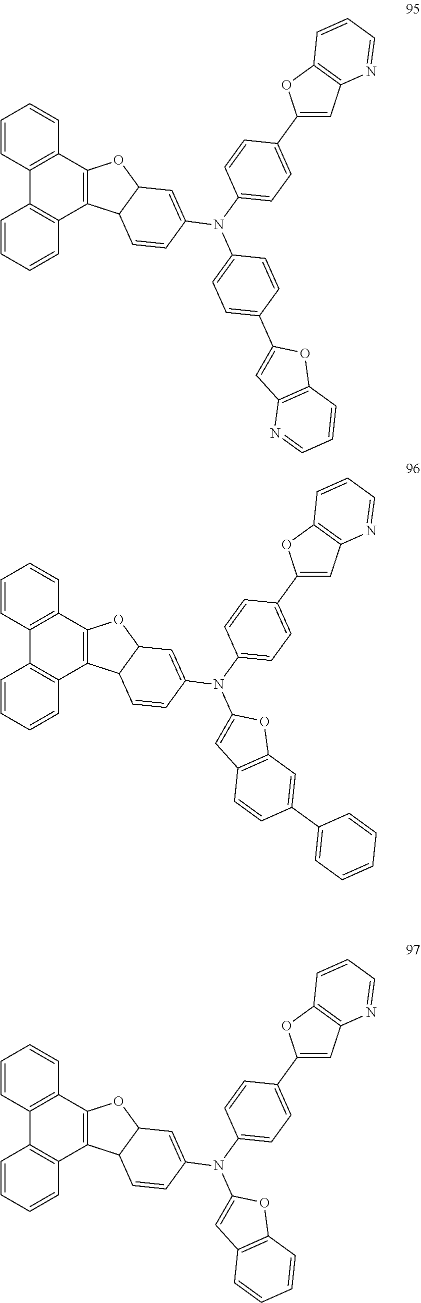

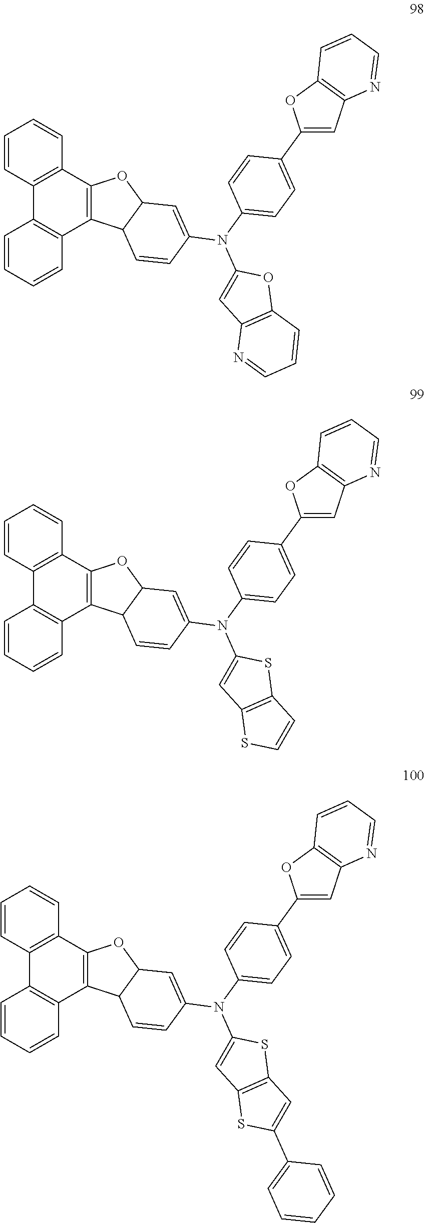 [SMILES: C1=CC2c3c(c4ccccc4c4ccccc34)OC2C=C1N(c1ccc(-c2cc3ncccc3o2)cc1)c1cc2ncccc2o1 ; C1=CC2c3c(c4ccccc4c4ccccc34)OC2C=C1N(c1ccc(-c2cc3ncccc3o2)cc1)c1cc2sccc2s1 ; C1=CC2c3c(c4ccccc4c4ccccc34)OC2C=C1N(c1ccc(-c2cc3ncccc3o2)cc1)c1cc2sc(-c3ccccc3)cc2s1]

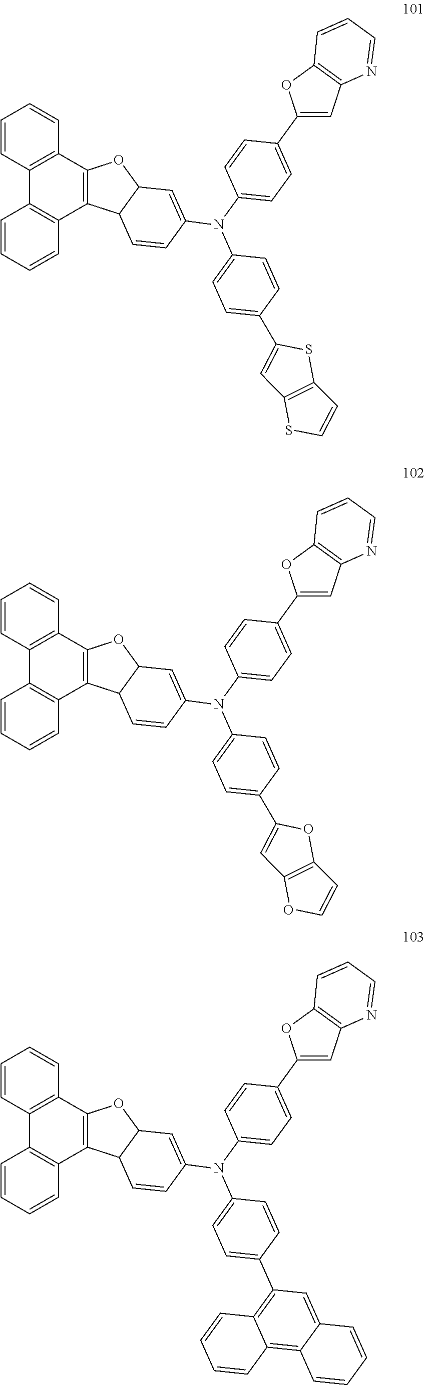 [SMILES: C1=CC2c3c(c4ccccc4c4ccccc34)OC2C=C1N(c1ccc(-c2cc3ncccc3o2)cc1)c1ccc(-c2cc3sccc3s2)cc1 ; C1=CC2c3c(c4ccccc4c4ccccc34)OC2C=C1N(c1ccc(-c2cc3ncccc3o2)cc1)c1ccc(-c2cc3occc3o2)cc1 ; C1=CC2c3c(c4ccccc4c4ccccc34)OC2C=C1N(c1ccc(-c2cc3ncccc3o2)cc1)c1ccc(-c2cc3ccccc3c3ccccc23)cc1]

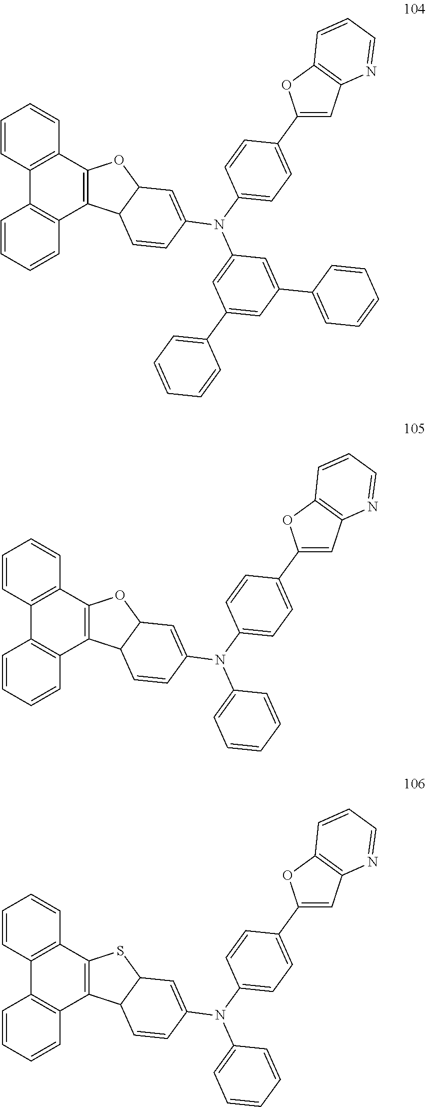 [SMILES: C1=CC2c3c(c4ccccc4c4ccccc34)OC2C=C1N(c1ccc(-c2cc3ncccc3o2)cc1)c1cc(-c2ccccc2)cc(-c2ccccc2)c1 ; C1=CC2c3c(c4ccccc4c4ccccc34)OC2C=C1N(c1ccccc1)c1ccc(-c2cc3ncccc3o2)cc1 ; C1=CC2c3c(c4ccccc4c4ccccc34)SC2C=C1N(c1ccccc1)c1ccc(-c2cc3ncccc3o2)cc1]

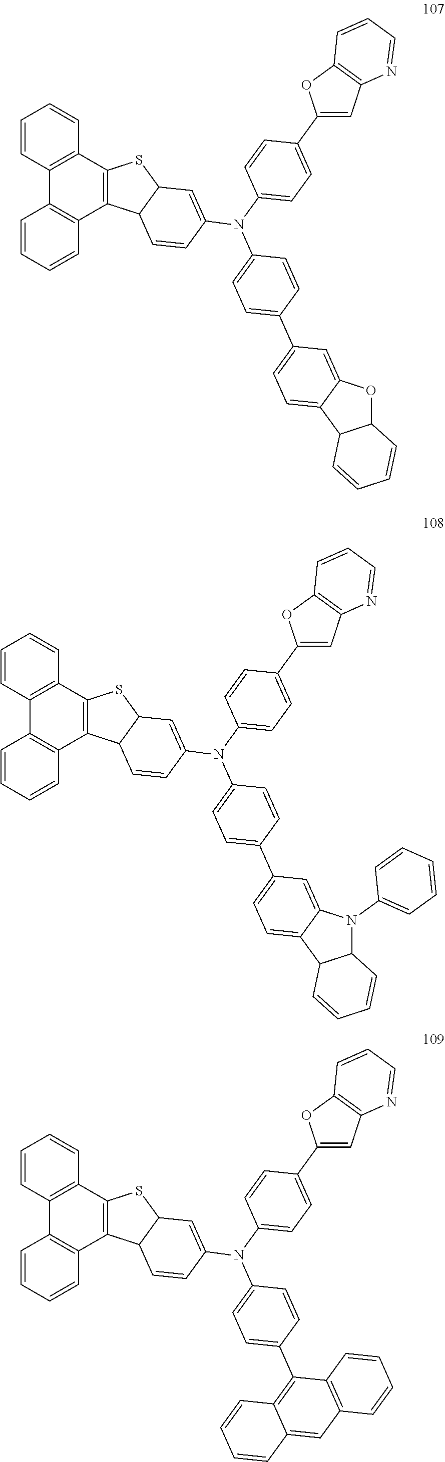

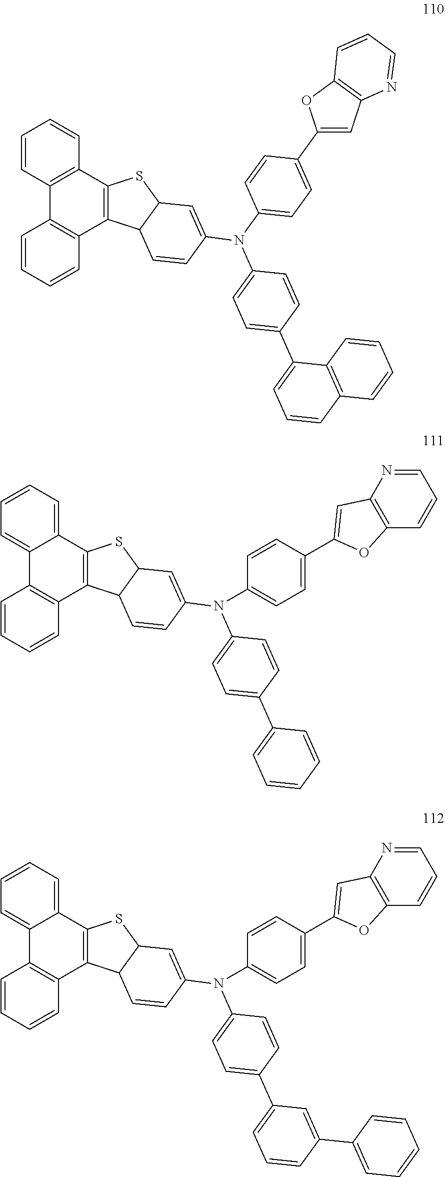 [SMILES: C1=CC2c3c(c4ccccc4c4ccccc34)SC2C=C1N(c1ccc(-c2cc3ncccc3o2)cc1)c1ccc(-c2cccc3ccccc23)cc1 ; C1=CC2c3c(c4ccccc4c4ccccc34)SC2C=C1N(c1ccc(-c2ccccc2)cc1)c1ccc(-c2cc3ncccc3o2)cc1 ; C1=CC2c3c(c4ccccc4c4ccccc34)SC2C=C1N(c1ccc(-c2cccc(-c3ccccc3)c2)cc1)c1ccc(-c2cc3ncccc3o2)cc1]

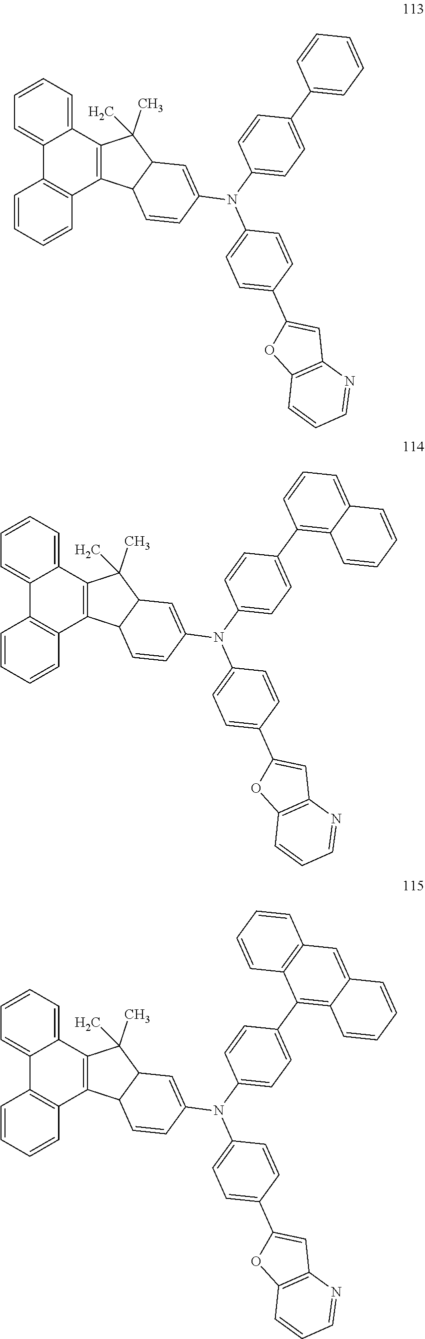

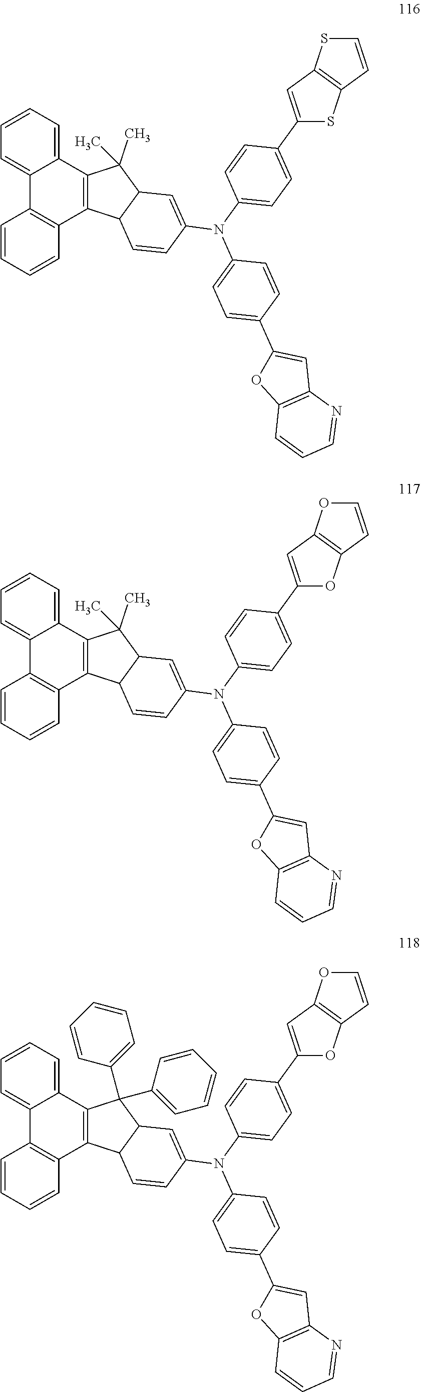 [SMILES: CC1(C)c2c(c3ccccc3c3ccccc23)C2C=CC(N(c3ccc(-c4cc5ncccc5o4)cc3)c3ccc(-c4cc5sccc5s4)cc3)=CC21 ; CC1(C)c2c(c3ccccc3c3ccccc23)C2C=CC(N(c3ccc(-c4cc5ncccc5o4)cc3)c3ccc(-c4cc5occc5o4)cc3)=CC21 ; C1=CC2c3c(c4ccccc4c4ccccc34)C(c3ccccc3)(c3ccccc3)C2C=C1N(c1ccc(-c2cc3ncccc3o2)cc1)c1ccc(-c2cc3occc3o2)cc1]

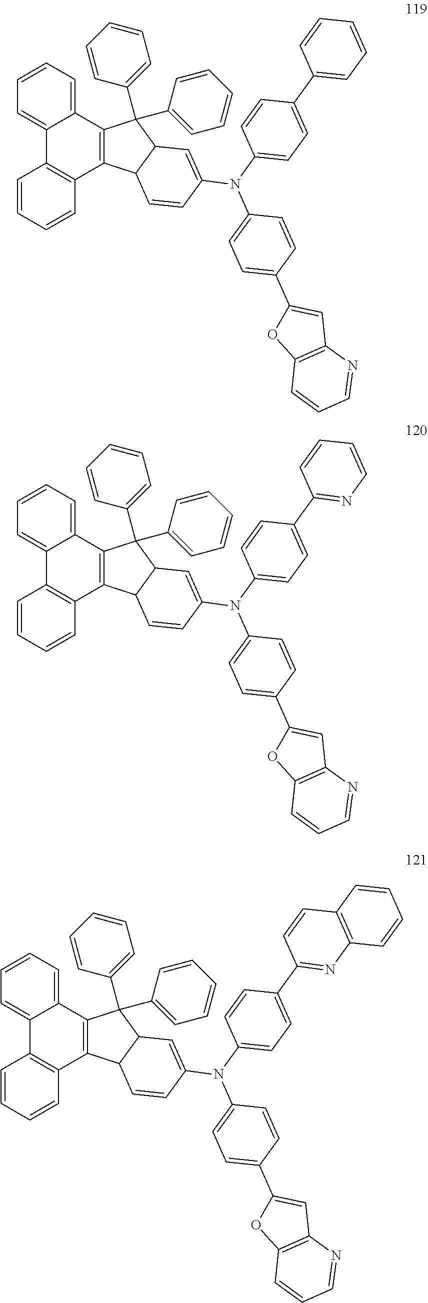

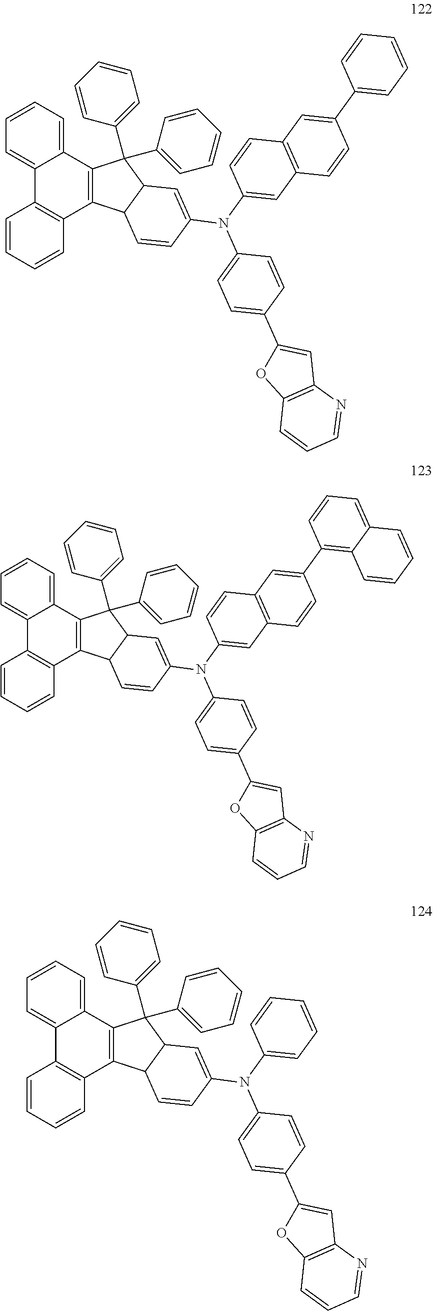 [SMILES: C1=CC2c3c(c4ccccc4c4ccccc34)C(c3ccccc3)(c3ccccc3)C2C=C1N(c1ccc(-c2cc3ncccc3o2)cc1)c1ccc2cc(-c3ccccc3)ccc2c1 ; C1=CC2c3c(c4ccccc4c4ccccc34)C(c3ccccc3)(c3ccccc3)C2C=C1N(c1ccc(-c2cc3ncccc3o2)cc1)c1ccc2cc(-c3cccc4ccccc34)ccc2c1 ; C1=CC2c3c(c4ccccc4c4ccccc34)C(c3ccccc3)(c3ccccc3)C2C=C1N(c1ccccc1)c1ccc(-c2cc3ncccc3o2)cc1]

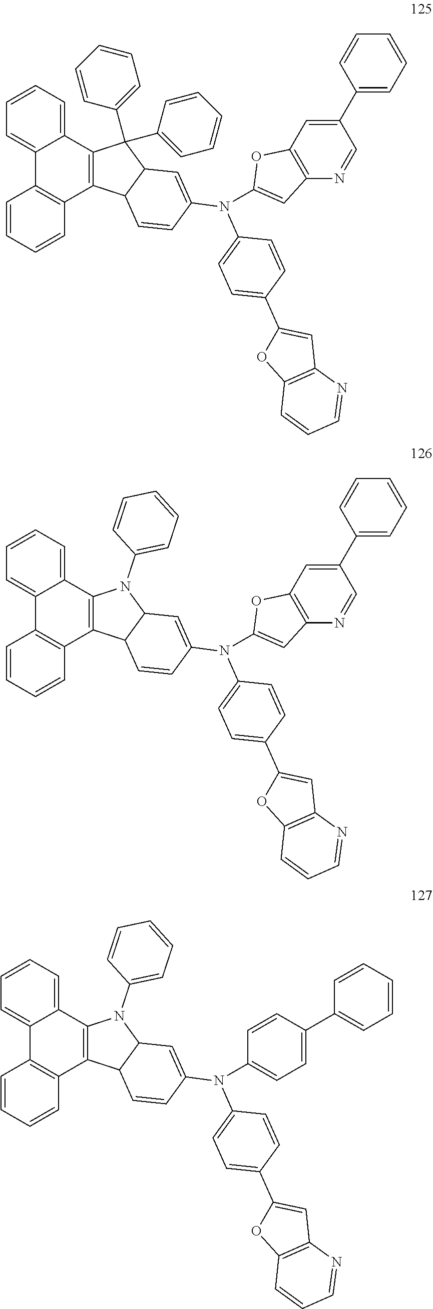 [SMILES: C1=CC2c3c(c4ccccc4c4ccccc34)C(c3ccccc3)(c3ccccc3)C2C=C1N(c1ccc(-c2cc3ncccc3o2)cc1)c1cc2ncc(-c3ccccc3)cc2o1 ; C1=CC2c3c(c4ccccc4c4ccccc34)N(c3ccccc3)C2C=C1N(c1ccc(-c2cc3ncccc3o2)cc1)c1cc2ncc(-c3ccccc3)cc2o1 ; C1=CC2c3c(c4ccccc4c4ccccc34)N(c3ccccc3)C2C=C1N(c1ccc(-c2ccccc2)cc1)c1ccc(-c2cc3ncccc3o2)cc1]

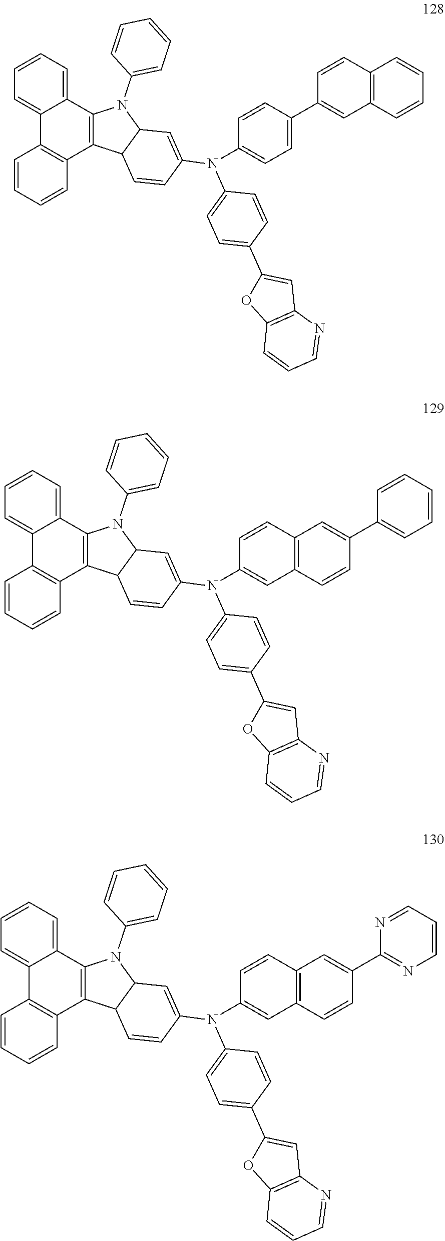 [SMILES: C1=CC2c3c(c4ccccc4c4ccccc34)N(c3ccccc3)C2C=C1N(c1ccc(-c2ccc3ccccc3c2)cc1)c1ccc(-c2cc3ncccc3o2)cc1 ; C1=CC2c3c(c4ccccc4c4ccccc34)N(c3ccccc3)C2C=C1N(c1ccc(-c2cc3ncccc3o2)cc1)c1ccc2cc(-c3ccccc3)ccc2c1 ; C1=CC2c3c(c4ccccc4c4ccccc34)N(c3ccccc3)C2C=C1N(c1ccc(-c2cc3ncccc3o2)cc1)c1ccc2cc(-c3ncccn3)ccc2c1]

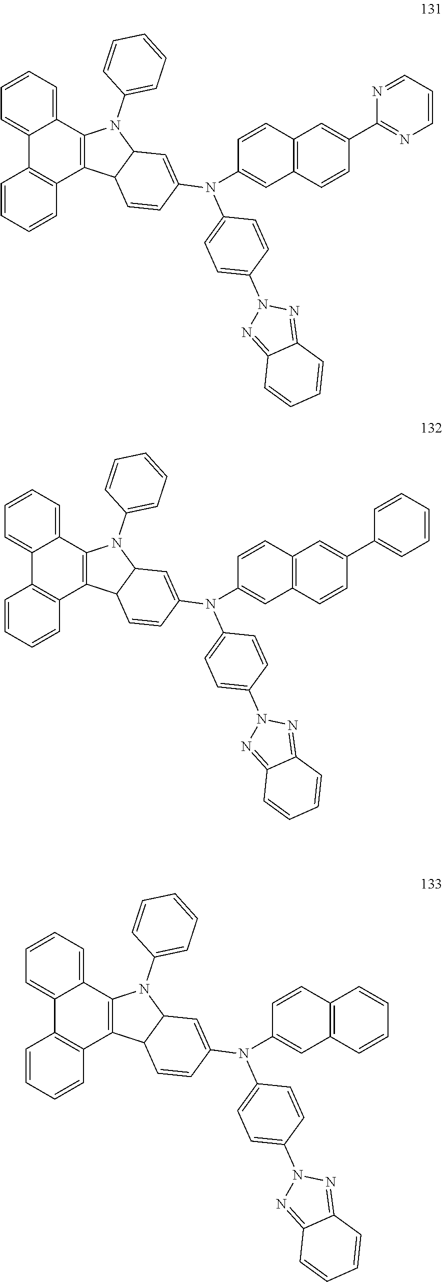 [SMILES: C1=CC2c3c(c4ccccc4c4ccccc34)N(c3ccccc3)C2C=C1N(c1ccc(-n2nc3ccccc3n2)cc1)c1ccc2cc(-c3ncccn3)ccc2c1 ; C1=CC2c3c(c4ccccc4c4ccccc34)N(c3ccccc3)C2C=C1N(c1ccc(-n2nc3ccccc3n2)cc1)c1ccc2cc(-c3ccccc3)ccc2c1 ; C1=CC2c3c(c4ccccc4c4ccccc34)N(c3ccccc3)C2C=C1N(c1ccc(-n2nc3ccccc3n2)cc1)c1ccc2ccccc2c1]

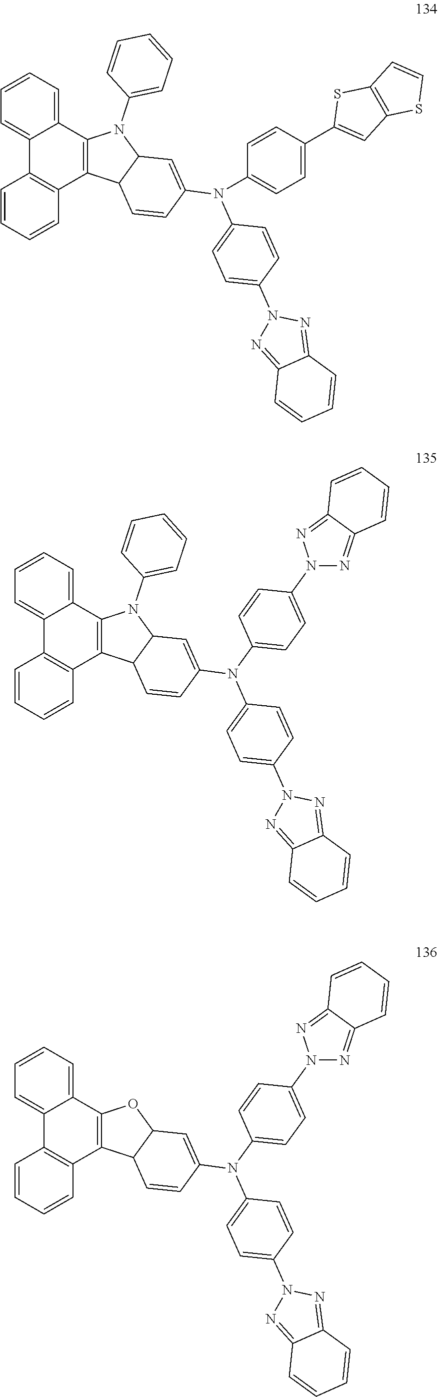 [SMILES: C1=CC2c3c(c4ccccc4c4ccccc34)N(c3ccccc3)C2C=C1N(c1ccc(-c2cc3sccc3s2)cc1)c1ccc(-n2nc3ccccc3n2)cc1 ; C1=CC2c3c(c4ccccc4c4ccccc34)N(c3ccccc3)C2C=C1N(c1ccc(-n2nc3ccccc3n2)cc1)c1ccc(-n2nc3ccccc3n2)cc1 ; C1=CC2c3c(c4ccccc4c4ccccc34)OC2C=C1N(c1ccc(-n2nc3ccccc3n2)cc1)c1ccc(-n2nc3ccccc3n2)cc1]

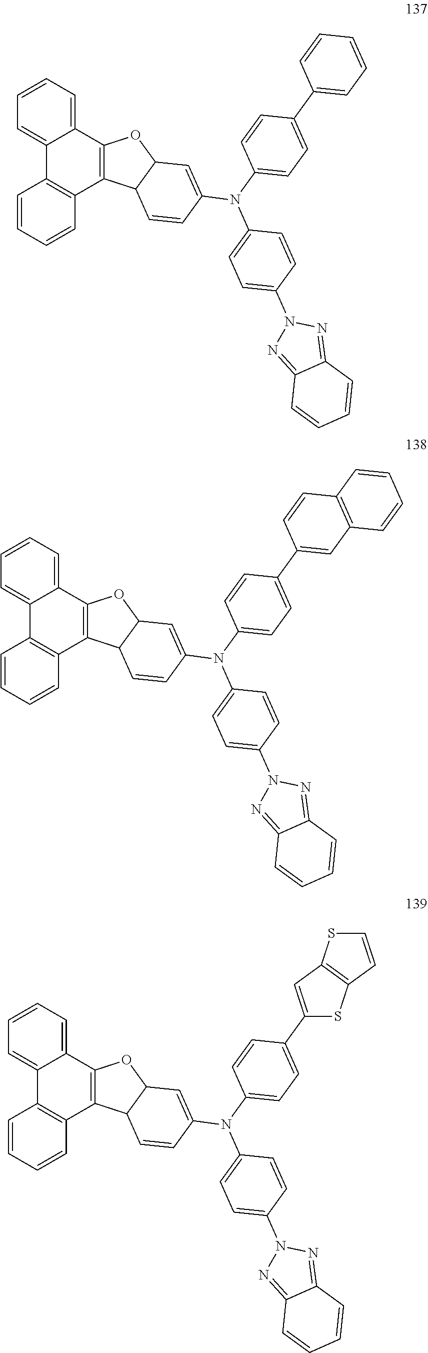 [SMILES: C1=CC2c3c(c4ccccc4c4ccccc34)OC2C=C1N(c1ccc(-c2ccccc2)cc1)c1ccc(-n2nc3ccccc3n2)cc1 ; C1=CC2c3c(c4ccccc4c4ccccc34)OC2C=C1N(c1ccc(-c2ccc3ccccc3c2)cc1)c1ccc(-n2nc3ccccc3n2)cc1 ; C1=CC2c3c(c4ccccc4c4ccccc34)OC2C=C1N(c1ccc(-c2cc3sccc3s2)cc1)c1ccc(-n2nc3ccccc3n2)cc1]

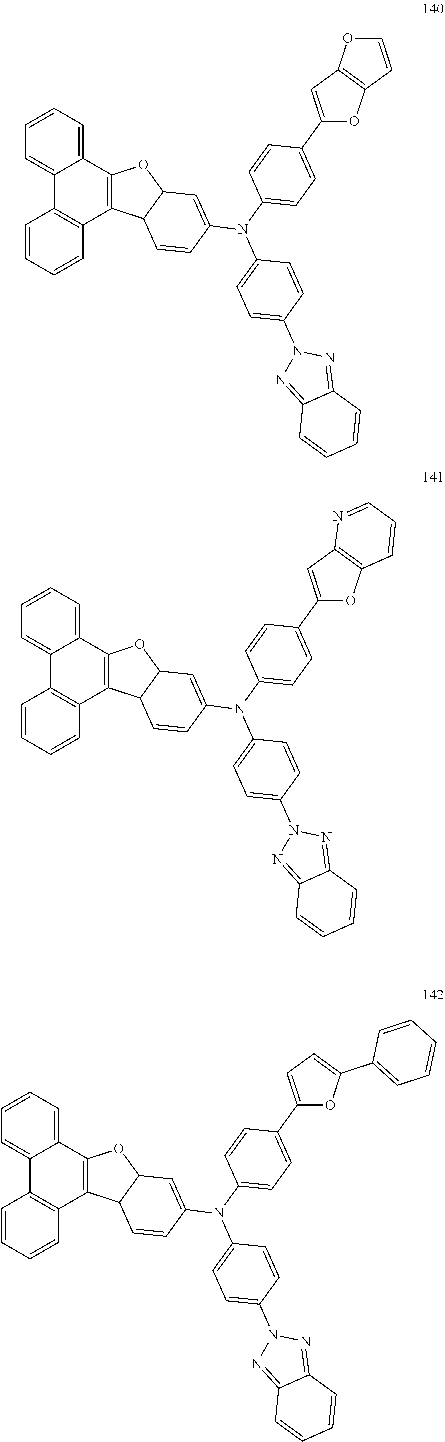 [SMILES: C1=CC2c3c(c4ccccc4c4ccccc34)OC2C=C1N(c1ccc(-c2cc3occc3o2)cc1)c1ccc(-n2nc3ccccc3n2)cc1 ; C1=CC2c3c(c4ccccc4c4ccccc34)OC2C=C1N(c1ccc(-c2cc3ncccc3o2)cc1)c1ccc(-n2nc3ccccc3n2)cc1 ; C1=CC2c3c(c4ccccc4c4ccccc34)OC2C=C1N(c1ccc(-c2ccc(-c3ccccc3)o2)cc1)c1ccc(-n2nc3ccccc3n2)cc1]

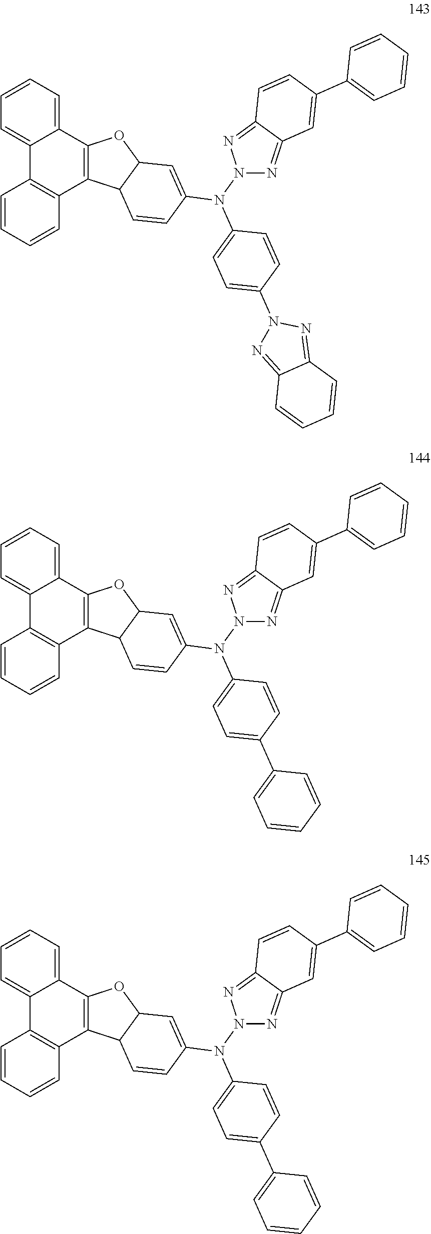 [SMILES: C1=CC2c3c(c4ccccc4c4ccccc34)OC2C=C1N(c1ccc(-n2nc3ccccc3n2)cc1)n1nc2ccc(-c3ccccc3)cc2n1 ; C1=CC2c3c(c4ccccc4c4ccccc34)OC2C=C1N(c1ccc(-c2ccccc2)cc1)n1nc2ccc(-c3ccccc3)cc2n1 ; C1=CC2c3c(c4ccccc4c4ccccc34)OC2C=C1N(c1ccc(-c2ccccc2)cc1)n1nc2ccc(-c3ccccc3)cc2n1]

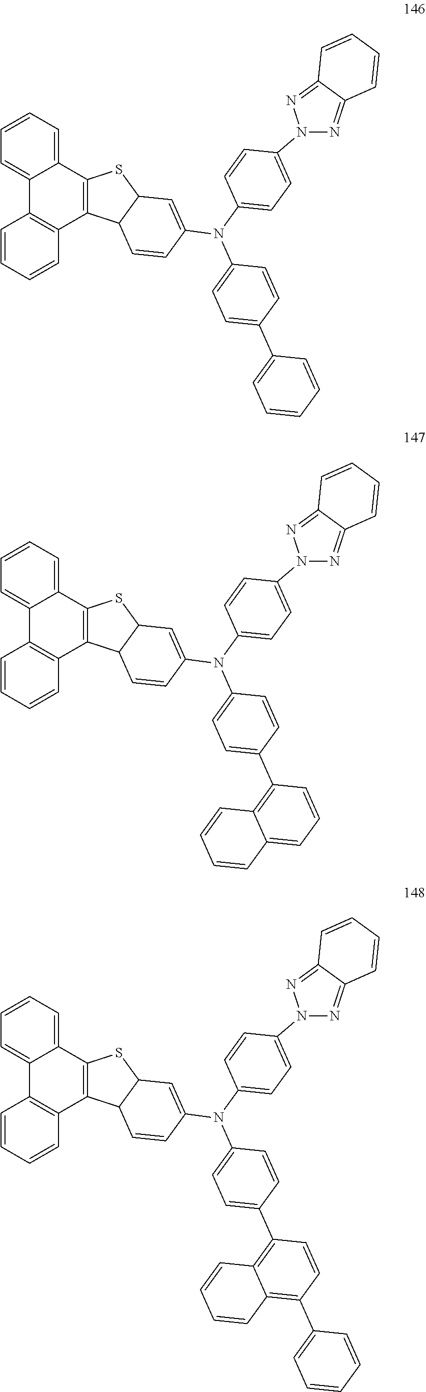 [SMILES: C1=CC2c3c(c4ccccc4c4ccccc34)SC2C=C1N(c1ccc(-c2ccccc2)cc1)c1ccc(-n2nc3ccccc3n2)cc1 ; C1=CC2c3c(c4ccccc4c4ccccc34)SC2C=C1N(c1ccc(-c2cccc3ccccc23)cc1)c1ccc(-n2nc3ccccc3n2)cc1 ; C1=CC2c3c(c4ccccc4c4ccccc34)SC2C=C1N(c1ccc(-c2ccc(-c3ccccc3)c3ccccc23)cc1)c1ccc(-n2nc3ccccc3n2)cc1]

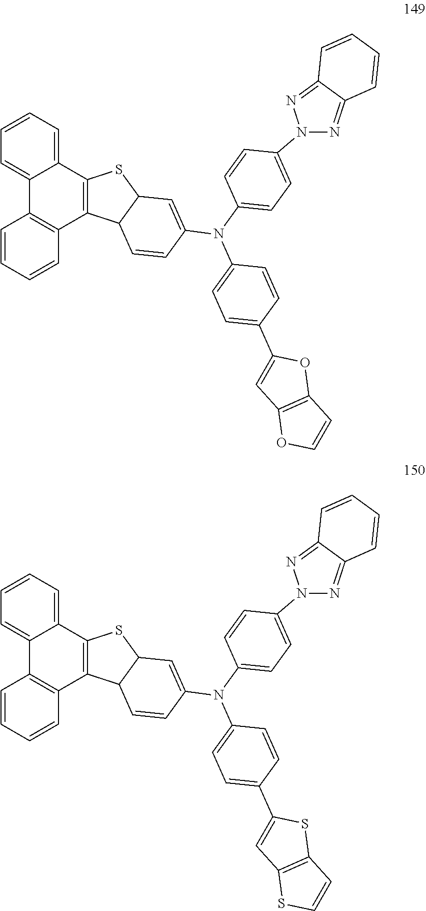 [SMILES: C1=CC2c3c(c4ccccc4c4ccccc34)SC2C=C1N(c1ccc(-c2cc3occc3o2)cc1)c1ccc(-n2nc3ccccc3n2)cc1 ; C1=CC2c3c(c4ccccc4c4ccccc34)SC2C=C1N(c1ccc(-c2cc3sccc3s2)cc1)c1ccc(-n2nc3ccccc3n2)cc1]

The embodiment of the present application further provides a method for preparing a compound of aromatic amines including a group of a derivative of phenanthrenes.

Taking a structure of compound 61 as an example, a preparation method is as follows:

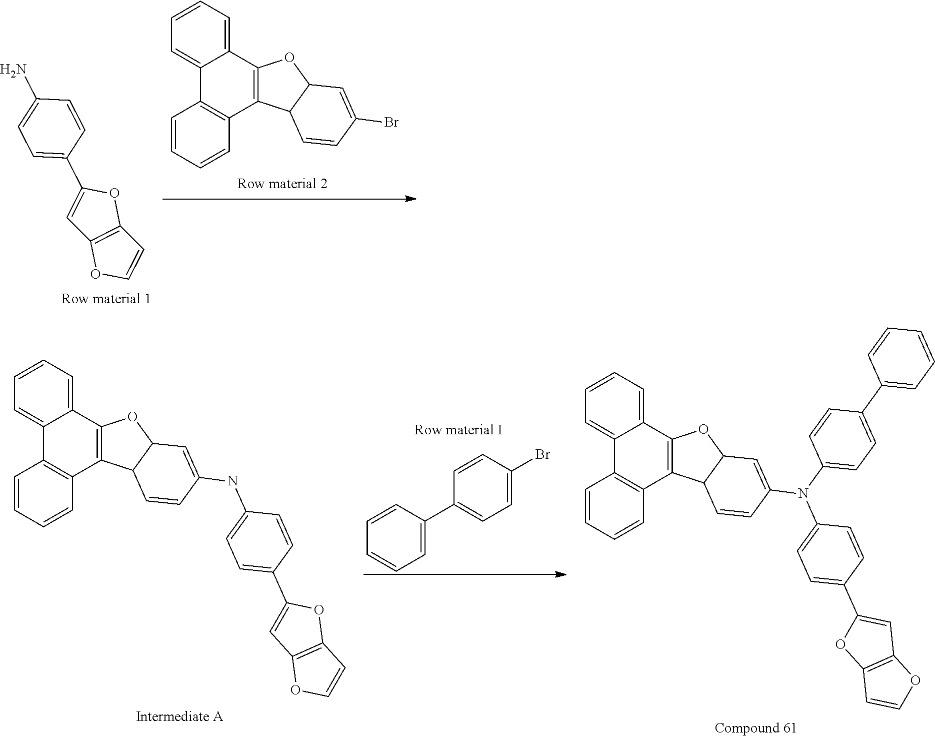 [SMILES: Nc1ccc(-c2cc3occc3o2)cc1 ; BrC1=CC2Oc3c(c4ccccc4c4ccccc34)C2C=C1 ; C1=CC2c3c(c4ccccc4c4ccccc34)OC2C=C1Nc1ccc(-c2cc3occc3o2)cc1 ; Brc1ccc(-c2ccccc2)cc1 ; C1=CC2c3c(c4ccccc4c4ccccc34)OC2C=C1N(c1ccc(-c2ccccc2)cc1)c1ccc(-c2cc3occc3o2)cc1]

Step 1: Synthetizing an intermediate A.

Adding 400 ml of toluene solvent into a reaction flask, and then respectively adding 0.02 mol of row material 1 and 0.02 mol of material 2, then adding 0.2 mol of sodium tert-butanol. After filling nitrogen gas, adding palladium acetate. Then refilling nitrogen gas, and adding 0.007 mol of toluene solution dissolved with tert-butylphosphine. After repeating filling nitrogen gas, performing a reflux reaction for 2 hours. After the reaction is completed, the mixture is cooled to a room temperature, and is filtered by diatomite to obtain a filtrate. After the filtrate is concentrated, methanol is added in the filtrate, then the filtrate is stood to recrystallize, and then recrystallization solid is obtained by filtering and by washing with methanol, that is, the intermediate A is obtained.

Step 2: Synthetizing the compound 61.

Adding 100 ml of toluene solvent into the reaction flask, and then successively adding 0.02 mol of raw material of the intermediate A, 0.07 mol of sodium tert-butanol and p-bromobiphenyl. After filling nitrogen gas, adding 0.1 g of palladium acetate. Then refilling nitrogen gas, and adding 0.007 mol of tert-butylphosphine. After repeating filling nitrogen gas, performing a reflux reaction for 2 hours. After the reaction is completed, the mixture is cooled to a room temperature, and is filtered by diatomite to obtain a filtrate. After the filtrate is concentrated, then heated. Adding a small amount of ethanol into the filtrate, then the filtrate is stood to recrystallize, and then recrystallization solid is obtained by filtering and by washing with ethanol, that is, a solid compound 61 is obtained.

A sample of the obtained compound 61 is tested and analyzed by mass spectrometry (MS) and hydrogen spectroscopy (1H NMR). A tested structure is as follows:

The mass spectrometry m/z: 617.20, element content (%): C₄₄H₂₇NO₃, C, 85.56%; H, 4.41%; N, 2.27%, and O, 7.77%.

1H NMR: 9.08-8.98 (2H), 8.17-8.03 (4H), 7.8-7.7 (4H), 7.68-7.62 (5H), 7.55 (2H), 7.49 (2H), 7.41-7.37 (5H), and 6.91-6.3 (3H).

Taking a structure of compound 65 as an example, the method for preparing the compound is as follows:

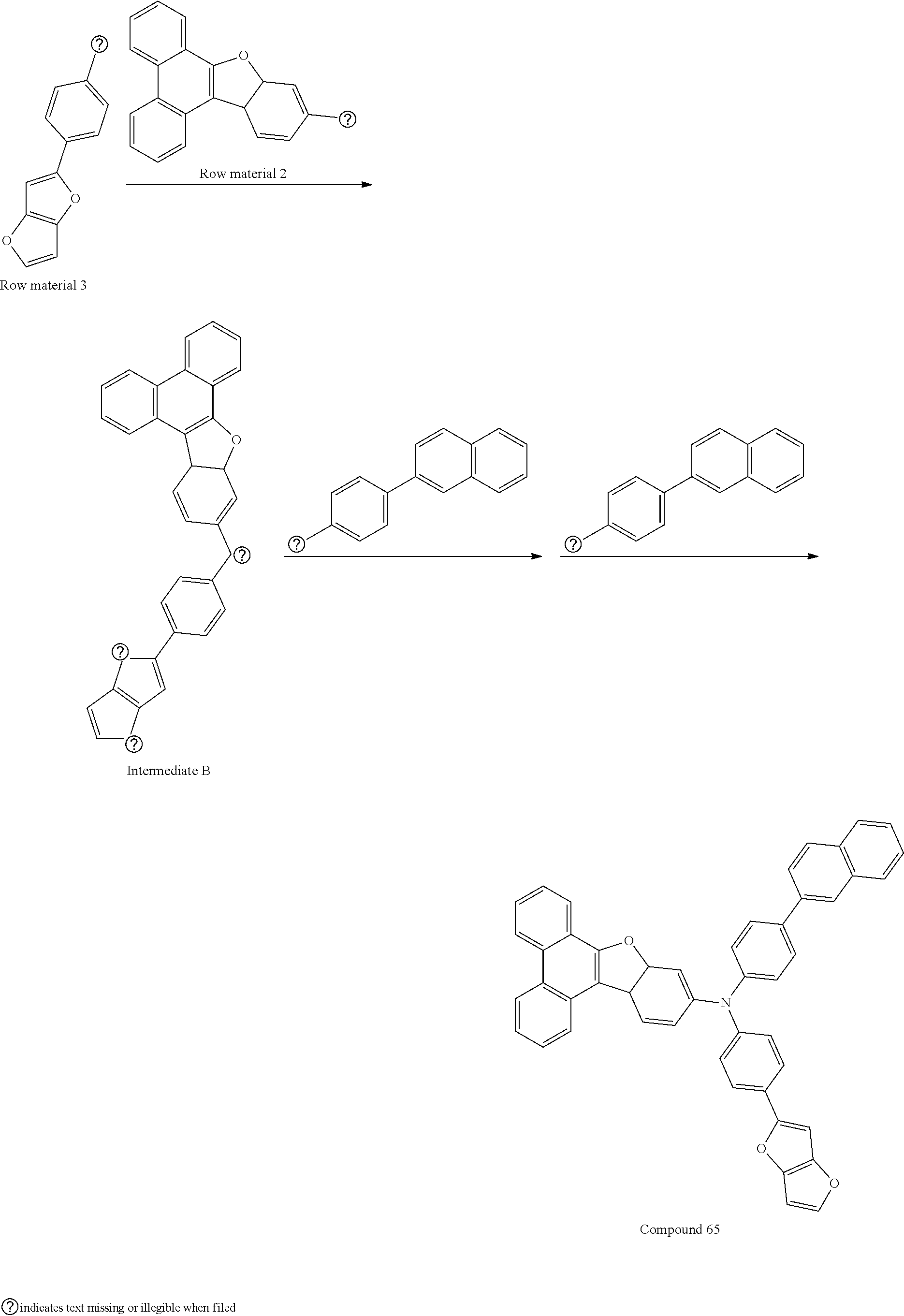

Among them, a synthesis method of intermediate B is similar to that of intermediate A. A synthesis method of the compound 65 is similar to that of the compound 61.

A sample of the obtained compound 65 is tested and analyzed by the mass spectrometry (MS) and the hydrogen spectroscopy (1H NMR). A tested structure is as follows:

The mass spectrometry m/z: 699.89, the element content (%): C₄₈H₂₉NOS₂, C, 82.37%; H, 4.18%. N, 2.00%; 2.29%; and S, 9.16%.

1H NMR: 9.08-8.98 (2H), 8.17-8.03 (5H), 7.99-7.7 (3H), 7.68-7.6 (5H), 7.55 (3H), 7.38-7.37 (5H), 7.31-7.1 (3H), and 6.99-6.91 (3H).

Taking a structure of compound 67 as an example, the method for preparing of the compound is as follows:

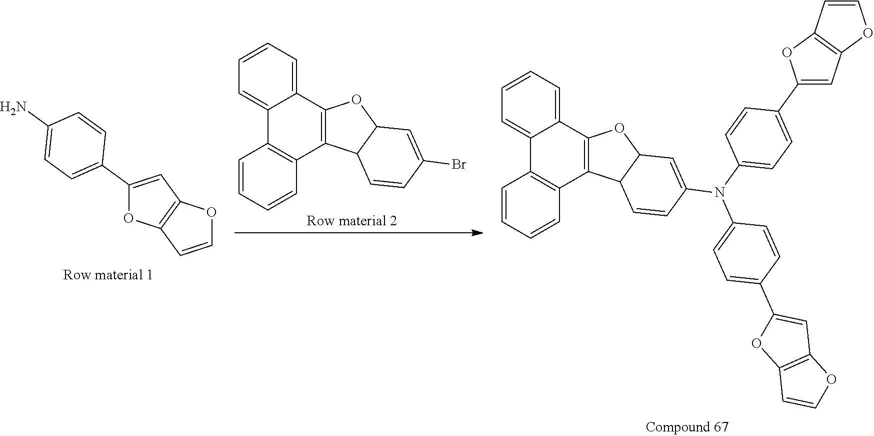

A sample of the obtained compound 67 is tested and analyzed by the mass spectrometry (NIS) and the hydrogen spectroscopy (1H NMR). A tested structure is as follows:

The mass spectrometry m/z: 647.69, the element content (%): C₄₄H₂₅NO₅, C, 81.6%; H, 3.89%; N, 2.16%; and O, 12.35%.

1H NMR: 9.08-8.98 (2H), 8.17-8.03 (5H), 7.8-7.7 (2H), 7.68-7.62 (7H), 7.37 (4H), and 6.91-6.3 (5H).

Taking a structure of compound 69 as an example, the method for preparing the compound is as follows:

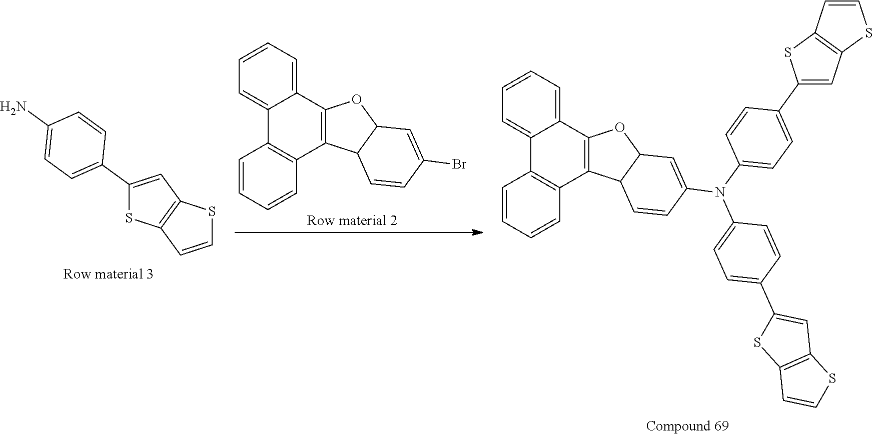

A sample of the obtained compound 69 is tested and analyzed by the mass spectrometry (MS) and the hydrogen spectroscopy (1H NMR). A tested structure is as follows:

The mass spectrum m/z: 711.93, the element content (%): C₄₄H₂₅NOS₄, C, 74.23%; H, 3.54%; N, 1.97%; 0, 2.25%; and S, 18.01%.

1H NMR: 9.08-8.98 (2H), 8.17-8.03 (3H), 7.8-7.7 (2H), 7.68-7.62 (3H), 7.37 (4H), 7.31 (2H), 7.12-7.1 (4H), and 6.99-6.91 (5H).

Taking a structure of compound 77 as an example, the method for preparing the compound is as follows:

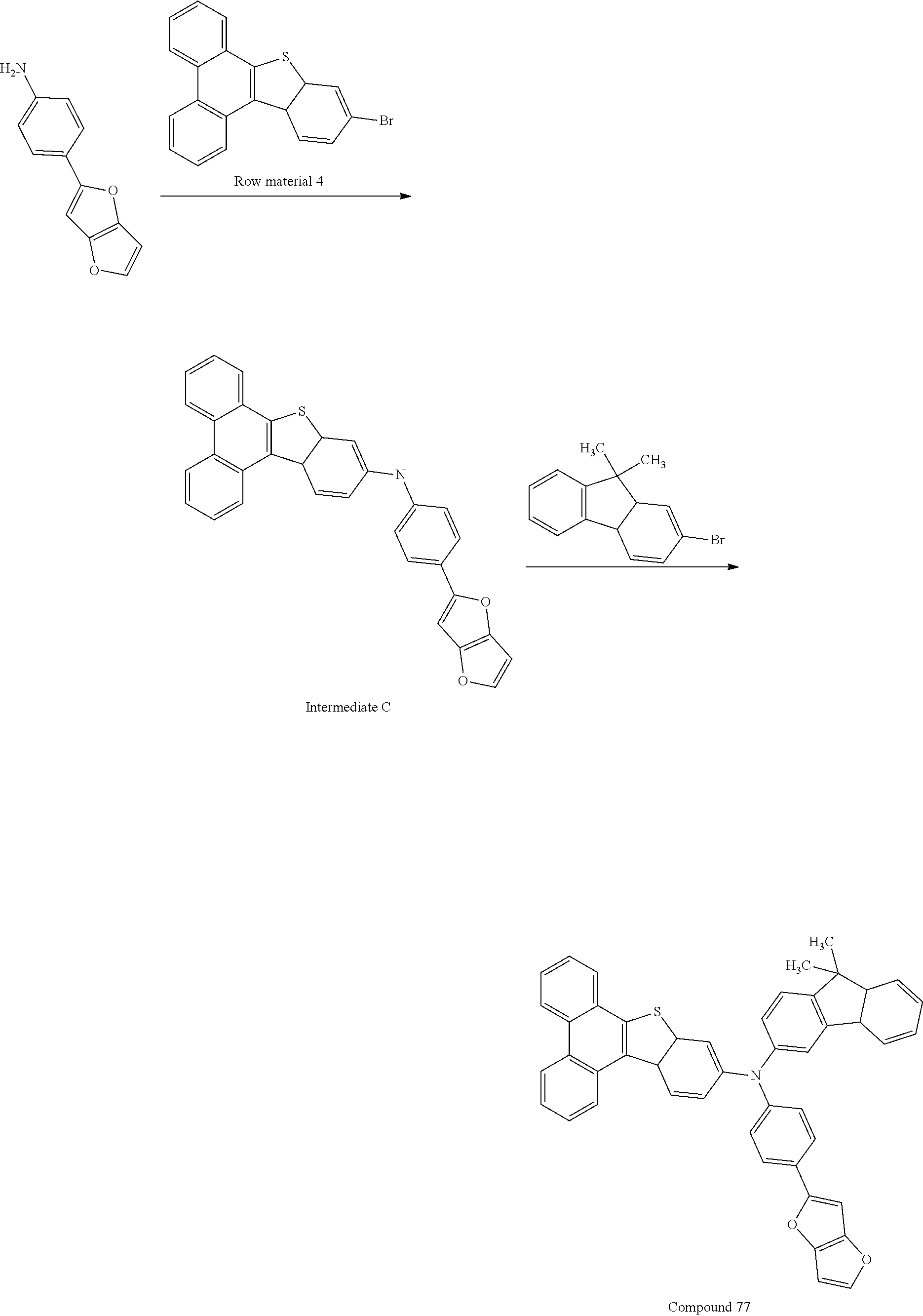 [SMILES: Nc1ccc(-c2cc3occc3o2)cc1 ; BrC1=CC2Sc3c(c4ccccc4c4ccccc34)C2C=C1 ; C1=CC2c3c(c4ccccc4c4ccccc34)SC2C=C1Nc1ccc(-c2cc3occc3o2)cc1 ; CC1(C)c2ccccc2C2C=CC(Br)=CC21 ; CC1(C)c2ccc(N(C3=CC4Sc5c(c6ccccc6c6ccccc56)C4C=C3)c3ccc(-c4cc5occc5o4)cc3)cc2C2C=CC=CC21]

A sample of the obtained compound 77 is tested and analyzed by the mass spectrometry (MS) and the hydrogen spectroscopy (1H NMR). A tested structure is as follows:

The mass spectrometry m/z: 673.83, the element content (%): C₄₇H₃₁NO₂S, C, 83.78%: H, 4.64%: N, 2.08%; O, 4.75%; and S, 4.76%.

1H NMR: 9.08-8.98 (2H), 8.17-8.01 (4H), 7.9-7.7 (2H), 7.68-7.62 (7H), 7.55-7.51 (2H), 7.43 (1H), 7.38-7.37 (3H), 7.28-06 (2H), 6.56-6.3 (2H), and 1.69 (6H).

Taking a structure of compound 89 as an example, the method for preparing the compound is as follows:

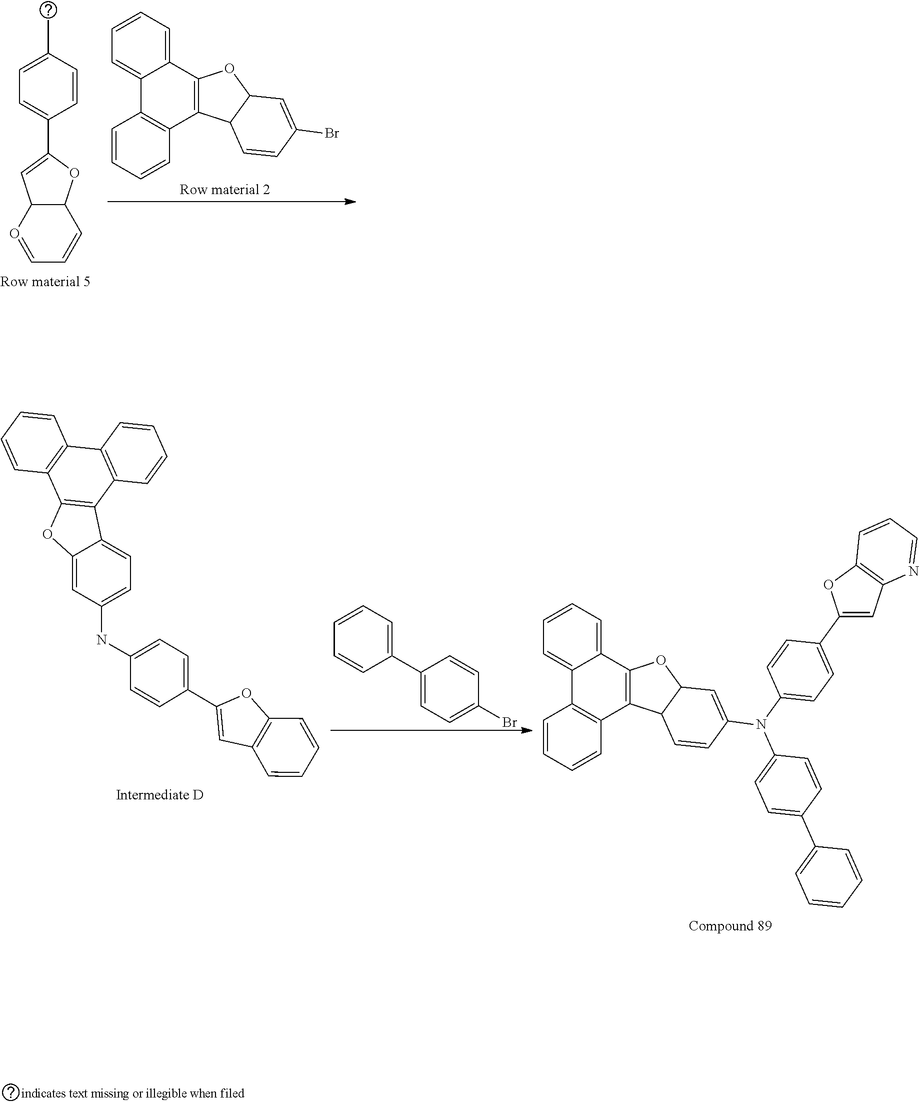

A sample of the obtained compound 89 is tested and analyzed by the mass spectrometry (MS) and the hydrogen spectroscopy (1H NMR). A tested structure is as follows:

The mass spectrometry m/z: 628.73, the element content (%): C₄₅H₂₈N₂O₂, C, 85.97%; H, 4.49%; N, 4.46%; and O, 5.09%.

1H NMR: 9.08-8.98 (2H), 8.43-8.03 (4H), 7.8-7.7 (4H), 7.68-7.62 (5H), 7.55-7.41 (5H), 7.37-7.21 (6H), and 6.91-6.56 (2H).

Taking a structure of compound 102 as an example, the method for preparing the compound is as follows:

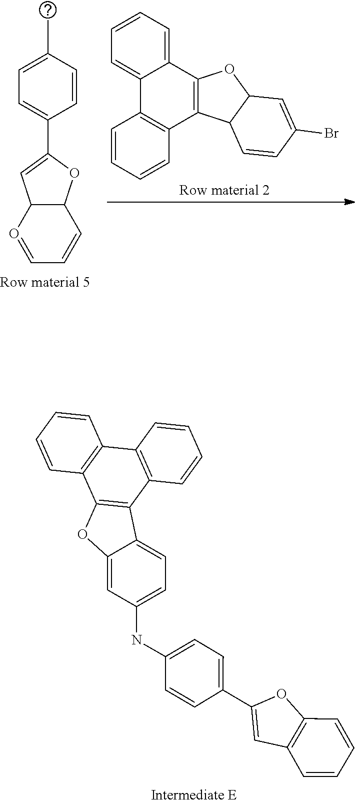

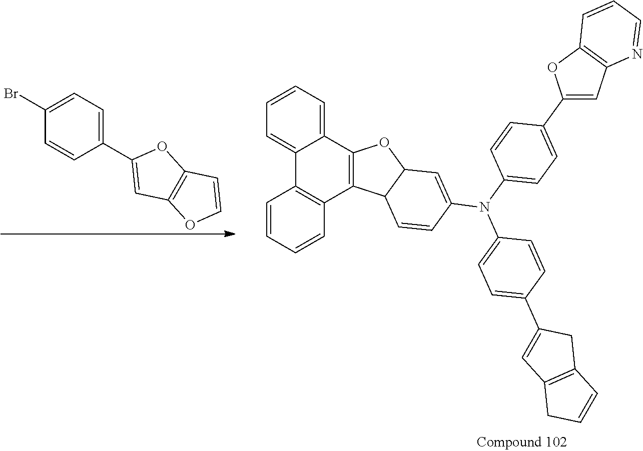

A sample of the obtained compound 102 is tested and analyzed by the mass spectrometry (MS) and the hydrogen spectroscopy (1H NMR). A tested structure is as follows:

The mass spectrometry m/z: 658.71, the element content (%): C₄₅H₂₆N₂O₄, C, 82.05%: H, 3.98%: N, 4.25%; and 0, 9.72%.

1H NMR: 9.08-8.98 (2H), 8.43-8.03 (5H), 7.8-7.7 (2H), 7.68-7.62 (7H), 7.37 (4H), 7.33-7.21 (2H), and 6.91-6.3 (4H).

Taking a structure of compound 135 as an example, the method for preparing the compound is as follows:

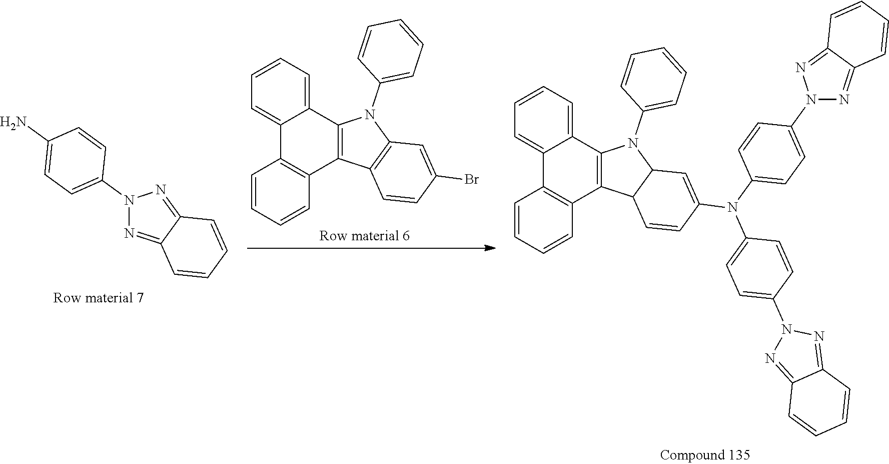

A sample of the obtained compound 135 is tested and analyzed by the mass spectrometry (MS) and the hydrogen spectroscopy (1H NMR). A tested structure is as follows:

The mass spectrum m/z: 744.86, the element content (%): C₅₀H₃₂N₈, C, 80.63%; H, 4.33%; and N, 15.04%.

1H NMR: 9.08-8.98 (2H), 8.39-8.03 (7H), 7.7 (1H), 7.68-7.62 (5H), 7.58-7.57 (5H), 7.54-7.5 (6H), 7.18 (5H), and 6.4 (1H).

In some embodiments of the present application, the glass transition temperature of the compound of aromatic amines ranges from 130° C. to 160° C.

Among them, the glass transition temperature (Tg) determines the thermal stability of the material during evaporation. The higher the Tg, the better the thermal stability of the material.

Table 4 provides the glass transition temperatures (Tg) of another part of compounds of aromatic amines provided by the embodiments of the application. Among them, the test equipment is a differential scanning calorimeter (DSC), and the test conditions are: the test atmosphere is nitrogen gas, the heating rate is 10° C./min, and the test temperature ranges from 50° C. to 300° C.

TABLE 4
Glass transition temperatures of compounds of aromatic amines
in related technology and of another part of the compounds of
aromatic amines provided by the embodiment of the application.

| | Tg ° C. |
|---|---|
| Ref1 (CP1) | 130 |
| Ref2 (CP2) | 125 |
| Compound 61 | 132 |
| Compound 62 | 132 |
| Compound 65 | 134 |
| Compound 69 | 132 |
| Compound 71 | 131 |
| Compound 75 | 133 |
| Compound 79 | 133 |
| Compound 80 | 134 |
| Compound 86 | 136 |
| Compound 90 | 134 |
| Compound 93 | 133 |
| Compound 95 | 131 |
| Compound 101 | 133 |
| Compound 103 | 135 |
| Compound 108 | 136 |
| Compound 111 | 132 |
| Compound 118 | 137 |
| Compound 120 | 136 |
| Compound 123 | 138 |
| Compound 136 | 133 |
| Compound 138 | 132 |
| Compound 140 | 134 |

Among them, the Ref1 and Ref2 are two light-extraction materials in related technology. According to data in the table 4, compared with the glass transition temperatures of the light-extraction materials in the related technology, the compounds of monoarylamines provided by the embodiments of the present application have higher glass transition temperatures. Therefore, in the process of preparing the capping layer by using the evaporation process, the process of preparing the capping layer, in which the material is the compound of monoarylamines provided by the embodiments of the present application, has better process stability.

In some embodiments of the present application, the refractive index of the compound of aromatic amines at a wavelength of 460 nm ranges from 2.0 to 2.2. The refractive index of the compound of aromatic amines at a wavelength of 530 nm ranges from 1.9 to 2.1. The refractive index of the compound of aromatic amines at a wavelength of 620 nm ranges from 1.85 to 2.0.

Among them, the refractive index is the important physical parameter of the material of the capping layer (CPL), and the size of the refractive index directly determines the light-coupling efficiency of the light-emitting device.

Table 5 provides the refractive indexes (n) of a part of the compounds of aromatic amines provided by the embodiments of the application, wherein the test equipment is the ellipsometer, and the test conditions are: the scanning range is from 245 nm to 1000 nm, the film of the compound is evaporated on the silicon chip, and the film has a thickness of 50 nm.

TABLE 5
the refractive indexes of compounds of aromatic amines in
related technology and of a part of the compounds of aromatic
amines provided by the embodiments of the application.

Refractive indexes n of different wavelengths

| Compound | 460 nm | 530 nm | 620 nm |
|---|---|---|---|
| Ref1 (CP1) | 1.93 | 1.86 | 1.83 |
| Ref [symbol] (CP [symbol] ) | 1.99 | 1.91 | 1.87 |
| Compound 61 | [symbol] .1 | 1.93 | 1.89 |
| Compound 62 | 2.1 [symbol] | 1.94 | [symbol] |
| Compound 65 | 2.15 | 1.96 | 1.92 |
| Compound 69 | 2.18 | 1.99 | 1.95 |
| Compound 71 | 2.17 | 1.96 | 1.93 |
| Compound 75 | 2.15 | 1.95 | 1.91 |
| Compound 79 | 2.11 | 1.92 | 1. [symbol] 9 |
| Compound 80 | 2.14 | 1.95 | 1.92 |
| Compound 86 | 2.13 | 1.94 | [symbol] |
| Compound 90 | 2.18 | 1.98 | 1.94 |
| Compound 93 | 2.16 | 1.96 | 1.91 |
| Compound 95 | 2.19 | 1.99 | 1.95 |
| Compound 101 | 2.17 | 1.96 | 1.93 |
| Compound 103 | 2.16 | 1.95 | 1.91 |
| Compound 108 | 2.17 | 1.95 | 1.93 |
| Compound 111 | 2.13 | 1.94 | 1.91 |
| Compound 118 | 2.17 | 1.96 | 1.93 |
| Compound 120 | 2.15 | 1.95 | 1.91 |
| Compound 123 | 2.17 | 195 | 1.9 [symbol] |
| Compound 136 | 2.19 | 1.99 | 1.96 |
| Compound 138 | 2.16 | 1.95 | 1.9 [symbol] |
| Compound 140 | 2.18 | 1.98 | 1.95 |

[symbol]

indicates data missing or illegible when filed

Among them, the Ref1 and Ref2 are two light-extraction materials in related technology. According to data in the table 5, compared with the refractive indexes of the light-extraction materials in the related technology, the compounds of monoarylamines provided is in the embodiments of the present application have higher refractive indexes (including but not limited to circumstances at wavelengths of 460 nm, 530 nm and 620 nm). Therefore, preparing the capping layer by using the compounds of monoarylamines provided in the embodiments of the present application is more conducive to coupling output of light in the light-emitting device, thus improving light-outlet efficiency of the light-emitting device.

In some embodiments of the present application, referring to that shown in FIG. 4, the light-emitting device includes a first electrode 2, a hole injection layer 3, a hole transport layer 4, an electron barrier layer 5, a light-emitting layer 6, a hole barrier layer 7, an electron transport layer 8, an electron injection layer 9, a second electrode 10 and a capping layer 11, which are arranged by overlapping in sequence. The first electrode 2 is an anode and the second electrode 10 is a cathode.

In some embodiments of the present application, a material of the hole injection layer 3 includes at least one of: a metal oxide, a material having hole transmission characteristics, and a P-type dopant. Materials of the hole transport layer 4 and the electron barrier layer 5 respectively include one of a compound of aromatic amines, a compound of dimethylfluorenes, and a compound of carbazoles. Materials of the hole barrier layer 7 and the electron transport layer 8 respectively include one of a derivative of imidazoles, a compound containing a structure of nitrogen six-membered ring, and a compound whose heterocycle has a substituent group of phosphine oxide series. A material of the electron injection layer 9 includes one of a metal or a metal compound.

In an exemplary embodiment, the material of the hole injection layer 3 may be an inorganic oxide, such as a molybdenum oxide, a titanium oxide, a vanadium oxide, a rhenium oxide, a ruthenium oxide, a chromium oxide, a zirconium oxide, a hafnium oxide, a tantalum oxide, a silver oxide, a tungsten oxide, or a manganese oxide.

In an exemplary embodiment, the material of the hole injection layer 3 may be a p-type dopant of strong electron absorption series, and a dopant of a hole transport material, for example, hexacyanohexa-azotriphenylene, or 2, 3, 5, 6-tetrafluoro-7, 7, 8, 8-tetracyano-p-quinone methane (F4TCNQ), or 1, 2, 3-tri [(cyano)(4-cyano-2, 3, 5, 6-tetrafluorophenyl)methylene] cyclopropane.

In an exemplary embodiment, the material of the hole transport layer 4 may be a material of aromatic amines with hole transmission characteristics, a material of dimethylfluorenes, or a material carbazoles, such as 4, 4′-bis [N-(1-naphthyl)-N-phenylamino]biphenyl (NPB): N, N′-bis (3-methylphenyl)-N, N′-diphenyl-[1, 1′-biphenyl]-4, 4′-diamine (TPD); 4-phenyl-4′-(9-phenylfluoren-9-yl) triphenylamine (BAFLP): 4, 4′-bis [N-(9, 9-dimethylfluoren-2-yl)-N-phenylamino] biphenyl (DFLDPBi); 4, 4′-bis (9-carbazolyl) biphenyl (CBP): or 9-phenyl-3-[4-(10-phenyl-9 anthracyl) phenyl]-9H-carbazole (PCzPA).

In an exemplary embodiment, the material of the electron barrier layer (Prime) 5 may be a material of aromatic amines with hole transmission characteristics, a material of dimethylfluorenes, or a material of carbazoles, such as 4, 4′-bis [N-(1-naphthyl)-N-phenylamino]biphenyl (NPB): N. N′-bis (3-methylphenyl)-N, N′-diphenyl-[1, 1′-biphenyl]-4, 4′-diamine (TPD); 4-phenyl-4′-(9-phenylfluoren-9-yl) triphenylamine (BAFLP): 4, 4′-bis [N-(9, 9-dimethylfluoren-2-yl)-N-phenylamino] biphenyl (DFLDPBi): 4, 4′-bis (9-carbazolyl) biphenyl (CBP); and 9-phenyl-3-[4-(10-phenyl-9 anthracyl)phenyl]-9H-carbazole (PCzPA).

In an exemplary embodiment, a light-emitting color of the light-emitting layer 6 may be blue.

For example, a light-emitting material for blue may include a derivative of pyrenes, a derivative of anthracenes, a derivative of fluorenes, a derivative of perylenes, a derivative of styrylamines, and a metal complex, etc. For example, N1, N6-bis ([1, 1′-biphenyl]-2-yl)-N1, N6-bis ([1, 1′-biphenyl]-4-yl) pyrene-1, 6-diamine; 9, 10-bis-(2-naphthyl) anthracene (ADN); 2-methyl-9, 10-di-2-naphthylanthracene (MADN): 2, 5, 8, 11-tetra-tert-butyl perylene (TBPe): 4, 4′-bis [4-(diphenylamino) styryl] biphenyl (BDAV Bi): 4, 4′-bis [4-(diphenylamino) styrene]biphenyl (DPAVBi); and bis (4, 6-difluorophenylpyridine-C2, N) pyridineformylIridium (FIrpic).

In an exemplary embodiment, the light-emitting color of the light-emitting layer 6 may be green.

In an exemplary embodiment, a light-emitting material for green may include any one or more of a coumarin dye, a light-emitting material for green of derivatives of quinacridone copper, a light-emitting material for green of polycyclic aromatic hydrocarbons, a light-emitting material for green of derivatives of diamine anthracenes, a light-emitting material for green of derivatives of carbazole, and a light-emitting material for green of metal complexes.

For example, the light-emitting material for green may include any one or more of: coumarin 6 (C-6); coumarin 545T (C-525T): quinacridone copper (QA); N, N′-dimethylquinacridone (DMQA); 5, 12-diphenylnaphthalene (DPT): N10, N10′-diphenyl-N10, N10′-diphenyl benzoyl-9, 9′-anthracene-10, 10′-diamine (BA-NPB for short); tri (8-hydroxyquinoline) aluminum (III) (Alq3 for shirt), tri (2-phenylpyridine)Iridium (Ir(ppy)3); and acetylpyruvate bis (2-phenylpyridine)Iridium(Ir(ppy)2(acac)).

In an exemplary embodiment, the light-emitting color of the light-emitting layer 6 may be red.

In an exemplary embodiment, a light-emitting material for red may include any one or more of: a light-emitting material for red of DCM series, and a light-emitting material for red of metal complexes.

For example, the light-emitting material for red may include any one or more of: 4-(dicyanomethylene)-2-methyl-6-(4-dimethylaminostyryl)-4H-pyran (DCM): 4-(dicyanomethyl)-2-tert-butyl-6-(1, 1, 7, 7-tetramethyljuronidine-9-alkenyl)-4H-pyran (DCJTB): bis (1-phenylisoquinoline)(acetylacetone)Iridim(III) (Ir(piq)2(acac)); octaethyl porphyrin platinum (PtOEP for short), and bis (2-(2′-benzothiophenyl) pyridine-N, C3′Xacetylacetone)Iridium complex (Ir(btp)2(acac) for short).

In an exemplary embodiment, the hole barrier layer 7 and the electron transport layer 8 may respectively be a compound of aromatic heterocyclic, for example, a derivative of imidazoles such as a derivative of benzimidazoles, a derivative of imidazolopyridines, and a derivative of benzimidazolphenanthridines; a derivative of zines such as a derivative of pyrimidines, and a derivative of triazines; a compound containing a nitrogen six-membered ring structure (as well as including a compound whose heterocycle has a substituent group of phosphine oxide series) such as a derivative of quinolines, a derivative of isoquinolines, and a derivative of phenanthrolines. Specifically, it may be 2-(4-tert-butylphenyl)-5-(4-tert-butylphenyl)-1, 3, 4-oxadiazole (PBD): 1, 3-bis [5-(p-tert-butylphenyl)-1, 3, 4-oxadiazole-2-yl] benzene (OXD-7): 3-(4-tert-butylphenyl)-4-phenyl-5-(4-biphenyl)-1, 2, 4-triazole (TAZ): 3-(4-tert-butylphenyl)-4-(4-ethylphenyl)-5-(4-biphenyl)-1, 2, 4-triazole (p-EtTAZ): phenanthroline (BPhen); (BCP); 4, 4′-bis (5-methylbenzoxazol-2-yl) stilbene (BzOs).

In an exemplary embodiment, the material of the electron injection layer 9 may be an alkali metal or a metal, for example, LiF, Yb Mg, Ca, an Yb-related compound, a Mg-related compound and a Ca-related compound.

In some embodiments of the present application, the light-emitting device further includes an encapsulation layer, and the encapsulation layer includes a glass encapsulation layer or a film encapsulation layer.

In a condition that the encapsulation layer is the film encapsulation layer, a transition layer is further set between the capping layer and the film encapsulation layer. A material of the transition layer is an organic material, and a refractive index of the material of the transition layer is less than the refractive index of the material of the capping layer.

In an exemplary embodiment, the refractive index of the material of the transition layer is less than or equal to 1.6.

In an exemplary embodiment, the material of the transition layer may further be LiF.

Specific structures of the light-emitting devices and corresponding tested data of the respective light-emitting devices are provided below.

A light-emitting device for blue:
The first electrode 2 (ITO), the hole injection layer 3 (m-MTDATA: F4TCNQ, 3%, 10 nm), the hole transport layer 4 (m-MTDATA, 100 nm), the electron barrier layer 5 (CBP, 10 nm), the light-emitting layer 6 (BH: BD, 5% BD, 20 nm), the hole barrier layer 7 (TPBI, 5 nm), the electron transport layer 8 (BCP: Liq, 1:1, 30 nm), the electron injection layer 9 (Yb, 1 nm), the second electrode 10 (Mg: Ag, 13 nm) and the capping layer 11 (CPL, 60 nm).

A light-emitting device for green:
The first electrode 2 (ITO), the hole injection layer 3 (m-MTDATA: F4TCNQ, 3%, 10 nm), the hole transport layer 4 (m-MTDATA, 100 am), the electron barrier layer 5 (CBP, 45 nm), the light-emitting layer 6 (BH: BD, 10% BD, 40 nm), the hole barrier layer 7 (TPBI, 5 nm), the electron transport layer 8 (BCP: Liq, 1:1, 30 nm), the electron injection layer 9 (Yb, Inn), the second electrode 10 (Mg: Ag, 13 nm) and the capping layer 11 (CPL, 60 nm).

A light-emitting device for red:
The first electrode 2 (ITO), the hole injection layer 3 (m-MTDATA: F4TCNQ, 3%, 10 nm), the hole transport layer 4 (m-MTDATA, 100 nm), the electron barrier layer 5 (CBP, 75 nm), the light-emitting layer 6 (BH: BD, 3% BD, 45 nm), the hole barrier layer 7 (TPBI, 5 nm), the electron transport layer 8 (BCP: Liq, 1:1, 30 nm), the electron injection layer 9 (Yb, 1 nm), the second electrode 10 (Mg: Ag, 13 nm) and the capping layer 11 (CPL, 60 nm).

Among them, structural formulas of materials of the respective film are as follows:

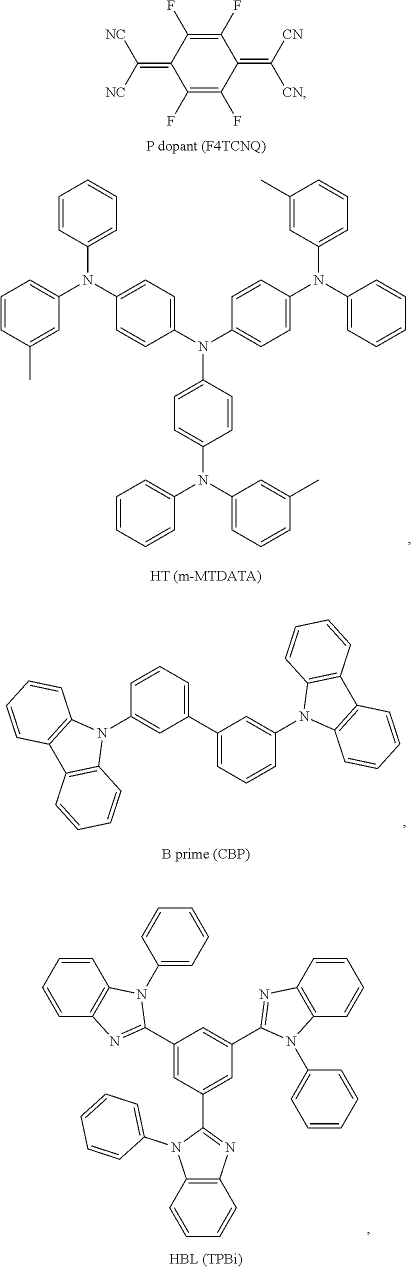 [SMILES: N#CC(C#N)=c1c(F)c(F)c(=C(C#N)C#N)c(F)c1F ; Cc1cccc(N(c2ccccc2)c2ccc(N(c3ccc(N(c4ccccc4)c4cccc(C)c4)cc3)c3ccc(N(c4ccccc4)c4cccc(C)c4)cc3)cc2)c1 ; c1cc(-c2cccc(-n3c4ccccc4c4ccccc43)c2)cc(-n2c3ccccc3c3ccccc32)c1 ; c1ccc(-n2c(-c3cc(-c4nc5ccccc5n4-c4ccccc4)cc(-c4nc5ccccc5n4-c4ccccc4)c3)nc3ccccc32)cc1]

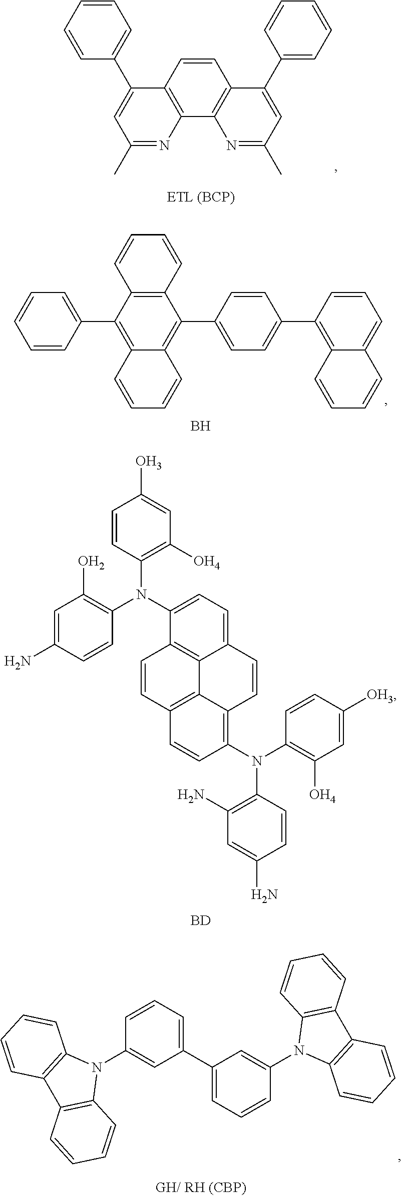

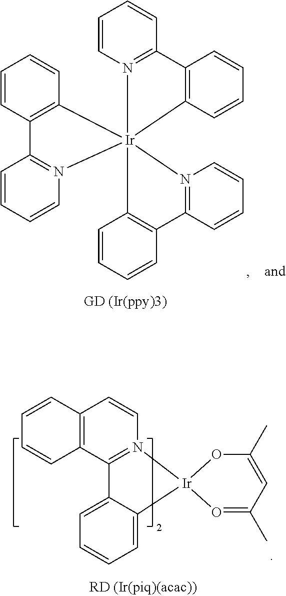

TABLE 6
performance test result 1 of the light-emitting device for blue

| | | | | Life |
|---|---|---|---|---|
| | CPL | Voltage | EQE | (LT95@ 1000 nit) |
| Reference 1 | CP1 | 100% | 100% | 100% |
| Reference 2 | CP2 | 99% | 102% | 103% |
| Embodiment 1 | Compound 1 | 99% | 109% | 110% |
| Embodiment 2 | Compound 2 | 98% | 106% | 107% |
| Embodiment 3 | Compound 5 | 100% | 108% | 107% |
| Embodiment 4 | Compound 8 | 99% | 103% | 103% |
| Embodiment 5 | Compound 11 | 100% | 103% | 104% |
| Embodiment 6 | Compound 13 | 98% | 104% | 106% |
| Embodiment 7 | Compound 17 | 97% | 107% | 108% |
| Embodiment 8 | Compound 23 | 98% | 107% | 106% |
| Embodiment 9 | Compound 25 | 99% | 108% | 109% |
| Embodiment 10 | Compound 28 | 98% | 110% | 108% |
| Embodiment 11 | Compound 32 | 99% | 112% | 110% |
| Embodiment 12 | Compound 38 | 99% | 109% | 107% |
| Embodiment 13 | Compound 40 | 100% | 112% | 109% |
| Embodiment 14 | Compound 43 | 100% | 111% | 108% |
| Embodiment 15 | Compound 48 | 100% | 108% | 106% |
| Embodiment 16 | Compound 50 | 101% | 106% | 105% |
| Embodiment 17 | Compound 52 | 98% | 105% | 105% |
| Embodiment 18 | Compound 54 | 98% | 105% | 106% |
| Embodiment 19 | Compound 59 | 99% | 108% | 108% |
| Embodiment 20 | Compound 60 | 99% | 115% | 110% |

TABLE 7
performance test result 2 of the light-emitting device for blue

| | | | | Life |
|---|---|---|---|---|
| | CPL | Voltage | EQE | (LT95@ 1000 nit) |
| Reference 1 | CP1 | 100% | 100% | 100% |
| Reference 2 | CP2 | 99% | 102% | 103% |
| Embodiment 21 | Compound 61 | 100% | 103% | 103% |
| Embodiment 22 | Compound 62 | 99% | 103% | 105% |
| Embodiment 23 | Compound 65 | 100% | 105% | 106% |
| Embodiment 24 | Compound 69 | 98% | 110% | 109% |
| Embodiment 25 | Compound 71 | 98% | 107% | 109% |
| Embodiment 26 | Compound 75 | 99% | 104% | 105% |
| Embodiment 27 | Compound 79 | 100% | 102% | 101% |
| Embodiment 28 | Compound 80 | 98% | 103% | 105% |
| Embodiment 29 | Compound 86 | 98% | 103% | 103% |
| Embodiment 30 | Compound 90 | 101% | 111% | 110% |
| Embodiment 31 | Compound 93 | 100% | 106% | 105% |
| Embodiment 32 | Compound 95 | 99% | 115% | 110% |
| Embodiment 33 | Compound 101 | 98% | 108% | 107% |
| Embodiment 34 | Compound 103 | 99% | 106% | 108% |
| Embodiment 35 | Compound 108 | 100% | 109% | 109% |
| Embodiment 36 | Compound 111 | 100% | 104% | 104% |
| Embodiment 37 | Compound 118 | 101% | 109% | 106% |
| Embodiment 38 | Compound 120 | 98% | 105% | 108% |
| Embodiment 39 | Compound 123 | 99% | 108% | 107% |
| Embodiment 40 | Compound 136 | 100% | 112% | 110% |
| Embodiment 41 | Compound 138 | 97% | 105% | 107% |
| Embodiment 42 | Compound 140 | 98% | 111% | 110% |

According to data in the table 6 and the table 7, compared with the light-emitting device for blue made of materials of CP1 and CP2 in related technology, the capping layer made of a compound of aromatic amines provided in the embodiment of the application has better light-extraction effect and high stability, which improves light-emitting efficiency (EQE) of the light-emitting device for blue, prolongs service life of the device, and makes change in voltage of the device little.

TABLE 8
performance test result of the light-emitting device for green

| | | | | Life |
|---|---|---|---|---|
| | CPL | Voltage | EQE | (LT95@ 10000 nit) |
| Reference 1 | CP1 | 100% | 100% | 100% |
| Reference 2 | CP2 | 100% | 101% | 103% |
| Embodiment 1 | Compound 1 | 100% | 107% | 108% |
| Embodiment 2 | Compound 2 | 99% | 104% | 105% |
| Embodiment 3 | Compound 5 | 99% | 106% | 105% |
| Embodiment 4 | Compound 8 | 99% | 102% | 103% |
| Embodiment 5 | Compound 11 | 99% | 102% | 103% |
| Embodiment 6 | Compound 13 | 98% | 103% | 104% |
| Embodiment 7 | Compound 17 | 98% | 105% | 104% |
| Embodiment 8 | Compound 23 | 97% | 105% | 105% |
| Embodiment 9 | Compound 25 | 98% | 106% | 107% |
| Embodiment 10 | Compound 28 | 99% | 107% | 105% |
| Embodiment 11 | Compound 32 | 99% | 109% | 108% |
| Embodiment 12 | Compound 38 | 100% | 107% | 106% |
| Embodiment 13 | Compound 40 | 100% | 109% | 109% |
| Embodiment 14 | Compound 43 | 99% | 109% | 108% |
| Embodiment 15 | Compound 48 | 100% | 106% | 105% |
| Embodiment 16 | Compound 50 | 99% | 104% | 103% |
| Embodiment 17 | Compound 52 | 99% | 103% | 104% |
| Embodiment 18 | Compound 54 | 100% | 103% | 105% |
| Embodiment 19 | Compound 59 | 98% | 106% | 106% |
| Embodiment 20 | Compound 60 | 98% | 112% | 111% |

According to data in the table 8, compared with the light-emitting device for green made of the materials of CP1 and CP2 in related technology, the capping layer made of a compound of aromatic amines provided in the embodiment of the application has better light-extraction effect and high stability, which improves light-emitting efficiency (EQE) of the light-emitting device for green, prolongs service life of the device, and makes change in voltage of the device little.

TABLE 9
performance test result of the light-emitting device for red

| | | | | Life |
|---|---|---|---|---|
| | CPL | Voltage | EQE | (LT95@ 5000 nit) |
| Reference 1 | CP1 | 100% | 100% | 100% |
| Reference 2 | CP2 | 100% | 101% | 102% |
| Embodiment 1 | Compound 1 | 99% | 106% | 106% |
| Embodiment 2 | Compound 2 | 100% | 103% | 104% |
| Embodiment 3 | Compound 5 | 98% | 105% | 104% |
| Embodiment 4 | Compound 8 | 99% | 102% | 102% |
| Embodiment 5 | Compound 11 | 98% | 101% | 103% |
| Embodiment 6 | Compound 13 | 98% | 103% | 102% |
| Embodiment 7 | Compound 17 | 97% | 104% | 104% |
| Embodiment 8 | Compound 23 | 97% | 103% | 104% |
| Embodiment 9 | Compound 25 | 99% | 105% | 106% |
| Embodiment 10 | Compound 28 | 100% | 106% | 105% |
| Embodiment 11 | Compound 32 | 99% | 107% | 106% |
| Embodiment 12 | Compound 38 | 100% | 106% | 104% |
| Embodiment 13 | Compound 40 | 100% | 108% | 108% |
| Embodiment 14 | Compound 43 | 98% | 108% | 106% |
| Embodiment 15 | Compound 48 | 99% | 105% | 103% |
| Embodiment 16 | Compound 50 | 99% | 103% | 103% |
| Embodiment 17 | Compound 52 | 99% | 102% | 103% |
| Embodiment 18 | Compound 54 | 100% | 103% | 104% |
| Embodiment 19 | Compound 59 | 98% | 105% | 104% |
| Embodiment 20 | Compound 60 | 98% | 110% | 109% |

According to data in the table 9, compared with the light-emitting device for red made of the materials of CP1 and CP2 in related technology, the capping layer made of a compound of aromatic amines provided in the embodiment of the application has better light-extraction effect and high stability, which improves light-emitting efficiency (EQE) of the light-emitting device for red, prolongs service life of the device, and makes change in voltage of the device little.

It should be noted that the EQE (external quantum efficiency) is a parameter to measure the light-emitting efficiency of the light-emitting device. LT95@5000 nit means a brightness value when a brightness attenuates to 95% of an initial brightness, in a condition that the initial brightness is 5000 nit, and LT95@1000 nit and LT95@10000 nit have a similar meaning, which will not be repeated herein.

In addition, specific data of the voltage, the EQE and the life in table 6-table 9 are not given. Data in reference 1 may be looked as 100%, and other data may be converted into percentage data for reference.

The embodiment of the present application further provides a display device, including the above light-emitting device.

Since the light-emitting device in the display device includes the above capping layer containing the compound of aromatic amines, its beneficial effect is similar to that described above, so it will not be repeated here.

The above are only specific implementations of the application, but the scope of protection of the application is not limited to this. Any ordinary skilled in the art may easily think of changes or replacements within the scope of technology disclosed in the application, which should be covered in the scope of protection of the application. Therefore, the scope of protection of the application shall be subject to the scope of protection of the claims.

1. A light-emitting device, comprising:
a substrate;
a first electrode and a second electrode, located on the substrate;
a light-emitting layer, located between the first electrode and the second electrode; and
a capping layer, located on a side of the second electrode away from the light-emitting layer, wherein the capping layer comprises a compound of aromatic amines, and a structure of the compound of aromatic amines comprises one of following two structures 1: 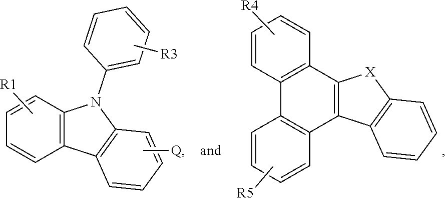
wherein the structure 1 is directly or indirectly bonded to a nitrogen atom in an amine of the compound of aromatic amines, the Q comprises the R2 or a R2-substituted aryl group, the X comprises one of C, O, N, NR6, and S, the R1, the R2, the R3, the R4, the R5 and the R6 respectively comprise one of: hydrogen, deuterium, halogen, a nitro group, a nitrile group, a substituted or unsubstituted C1 to C30 alkyl group, a substituted or unsubstituted C2 to C30 alkenyl group, a substituted or unsubstituted C1 to C30 alkoxy group, a substituted or unsubstituted C1 to C30 thioether group, a substituted or unsubstituted C6 to C50 aryl group, and a substituted or unsubstituted C2 to C50 heteroaryl group.

2. The light-emitting device according to claim 1, wherein the structure 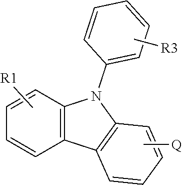
comprises one of:

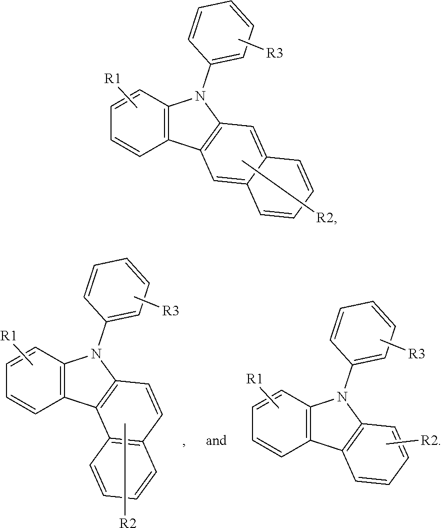

3. The light-emitting device according to claim 1, wherein the substituted or unsubstituted C2 to C50 heteroaryl group comprises a substituted or unsubstituted C2 to C50 heteroaryl group formed by a substituted or unsubstituted ring structure of C2 to C9.

4. The light-emitting device according to claim 1, wherein the substituted C1 to C30 alkyl group, the substituted C2 to C30 alkenyl group, the substituted C1 to C30 alkoxy group, the substituted C1 to C30 thioether group, the substituted C6 to C50 aryl group, and the substituted C2 to C50 heteroaryl group are referred to being replaced by one or more of following groups: deuterium, halogen, a nitro group, a nitrile group, a C1 to C30 alkyl group, a C2 to C30 alkenyl group, a C1 to C30 alkoxy group, a C1 to C30 thioether group, a C6 to C50 aromatic group, and a C2 to C50 heteroaryl group.

5. The light-emitting device according to claim 1, wherein the compound of aromatic amines comprises at least two heteroatoms.

6. The light-emitting device according to claim 2, wherein the compound of aromatic amines further comprises at least one of following four structures 2: 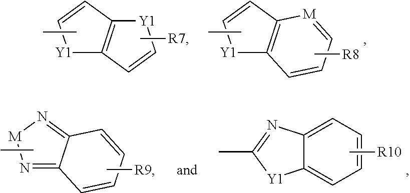
wherein the Y1 comprises O or S, the M comprises N or —CR11, and the R7, R8, R9, R10 and R11 respectively comprise one of: hydrogen, deuterium, halogen, a nitro group, a nitrile group, a substituted or unsubstituted C1 to C30 alkyl group, a substituted or unsubstituted C2 to C30 alkenyl group, a substituted or unsubstituted C1 to C30 alkoxy group, a substituted or unsubstituted C1 to C30 thioether group, a substituted or unsubstituted C6 to C50 aryl group, and a substituted or unsubstituted C2 to C50 heteroaryl group.

7. The light-emitting device according to claim 6, wherein a first position of one of the following three structures 1 is directly bonded with the nitrogen atom in the amine of the compound of aromatic amines, or
the first position of one of the following three structures 1 is bonded with the nitrogen atom in the amine of the compound of aromatic amines through L: 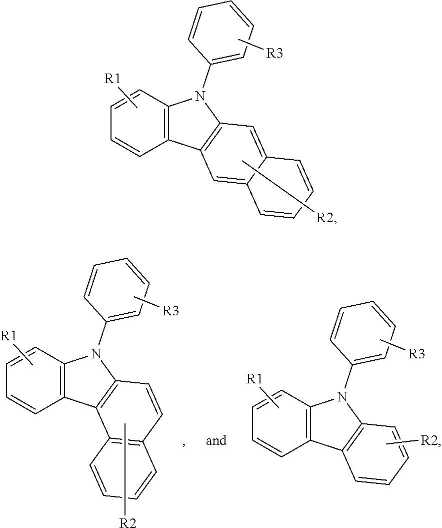
wherein the L is one of: the substituted or unsubstituted naphthyl group, a C6 to C50 arylidene group, and the substituted or unsubstituted C2 to C50 heteroaryl group, and
the first position is a position containing hydrogen or isotope of hydrogen.

8. The light-emitting device according to claim 6, wherein a second position of at least one of the following fourth structures 2 is directly bonded with the nitrogen atom in the amine of the compound of aromatic amines, or
the second position of at least one of the following fourth structures 2 is directly bonded with the nitrogen atom in the amine of the compound of aromatic amines through L: 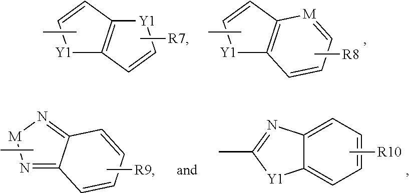
wherein the L is one of: the substituted or unsubstituted naphthyl group, the C6 to C50 arylidene group, and the substituted or unsubstituted C2 to C50 heteroaryl group, and
the second position is a position containing hydrogen or isotope of hydrogen.

9. The light-emitting device according to claim 1, wherein, in a condition that the compound of aromatic amines comprises the following structure 1, a general formula of the compound of aromatic amines is a following general formula IL 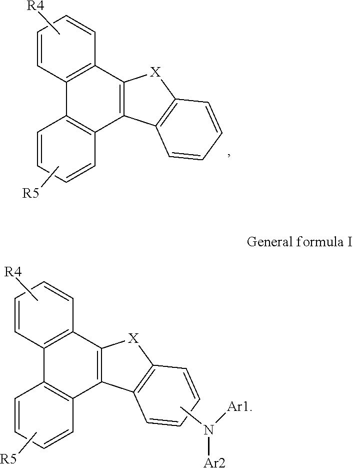
wherein a structure of at least one of the Ar1 and the Ar2 comprises one of following three structures 3: 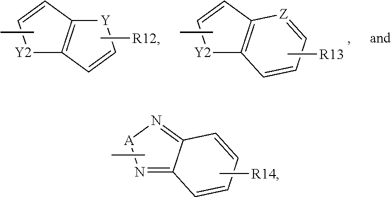
and
the structure 3 comprises at least one heteroatom.

10. The light-emitting device according to claim 9, wherein the Y2 comprises O, S or N, the Z comprises C or N, and the A comprises C or N, and
the R12, R13 and R14 respectively comprise one of: hydrogen, deuterium, halogen, a nitro group, a nitrile group, a substituted or unsubstituted C1 to C30 alkyl group, a substituted or unsubstituted C2 to C30 alkenyl group, a substituted or unsubstituted C1 to C30 alkoxy group, a substituted or unsubstituted C1 to C30 thioether group, a substituted or unsubstituted C6 to C50 aryl group, and a substituted or unsubstituted C2 to C50 heteroaryl group.

11. The light-emitting device according to claim 9, wherein the structure of another one of the Ar1 and Ar2 comprises any one of: a nitrile group, a C2 to C30 alkyl group, a C3 to C30 naphthyl group, a substituted or unsubstituted C6 to C60 aryl group, and a substituted or substituted C5 to C60 heteroaryl group.

12. The light-emitting device according to claim 1, wherein a glass transition temperature of the compound of aromatic amines ranges from 130° C. to 160° C.

13. The light-emitting device according to claim 1, wherein
a refractive index of the compound of aromatic amines at a wavelength of 460 nm ranges from 2.0 to 2.2,
the refractive index of the compound of aromatic amines at a wavelength of 530 nm ranges from 1.9 to 2.1, and
the refractive index of the compound of aromatic amines at a wavelength of 620 nm ranges from 1.85 to 2.0.

14. The light-emitting device according to claim 1, wherein the light-emitting device comprises: the first electrode, a hole injection layer, a hole transport layer, an electron barrier layer, the light-emitting layer, a hole barrier layer, an election transport layer, an electron injection layer, and the second electrode that are arranged by overlapping in sequence, and the first electrode is an anode and the second electrode is a cathode.

15. The light-emitting device according to claim 14, wherein a material of the hole injection layer comprises at least one of a metal oxide, a material having hole transmission characteristics, and a P-type dopant; materials of the hole transport layer and the electron barrier layer respectively comprise one of a compound of aromatic amines, a compound of dimethylfluorenes, and a compound of carbazoles; materials of the hole barrier layer and the electron transport layer respectively comprise one of a derivative of imidazoles, a compound containing a structure of nitrogen six-membered ring, and a compound whose heterocycle has a substituent group of phosphine oxide series, and a material of the electron injection layer comprises one of a metal or a metal compound.

16. The light-emitting device according to claim 1, further comprising an encapsulation layer, wherein the encapsulation layer comprises a glass encapsulation layer or a film encapsulation layer, and
in a condition that the encapsulation layer is the film encapsulation layer, a transition layer is further set between the capping layer and the film encapsulation layer, a material of the transition layer is an organic material, and a refractive index of the material of the transition layer is less than a refractive index of a material of the capping layer.

17. A display device, comprising the light-emitting device according to claim 1.

18. The display device according to claim 17, wherein
the structure 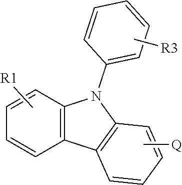
comprises one of:

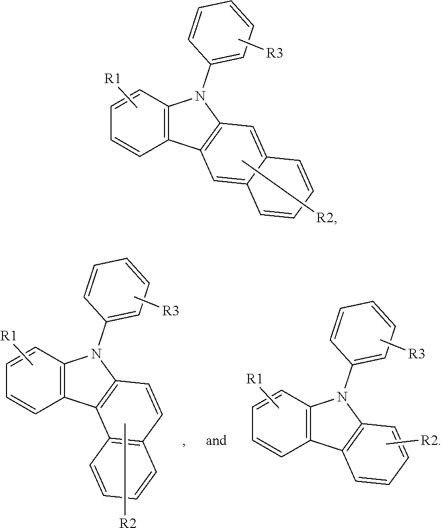

19. The display device according to claim 17, wherein the substituted or unsubstituted C2 to C50 heteroaryl group comprises a substituted or unsubstituted C2 to C50 heteroaryl group formed by a substituted or unsubstituted ring structure of C2 to C9.

20. The display device according to claim 17, wherein the substituted C1 to C30 alkyl group, the substituted C2 to C30 alkenyl group, the substituted C1 to C30 alkoxy group, the substituted C1 to C30 thioether group, the substituted C6 to C50 aryl group, and the substituted C2 to C50 heteroaryl group are referred to being replaced by one or more of following groups: deuterium, halogen, a nitro group, a nitrile group, a C1 to C30 alkyl group, a C2 to C30 alkenyl group, a C1 to C30 alkoxy group, a C1 to C30 thioether group, a C6 to C50 aromatic group, and a C2 to C50 heteroaryl group.
